(19) United States
(12) Patent Application Publication
(10) Pub. No.: US 2014/0148361 A1
(43) Pub. Date: May 29, 2014

(54) Generation and Expression of Engineered I-ONUI Endonuclease and Its Homologues and Uses Thereof

(71) Applicants: Barry L. Stoddard, Bellevue, WA (US); Abigail Rose Lambert, Kenmore, WA (US); Ryo Takeuchi, Seattle, WA (US); Andrew M. Scharenberg, Seattle, WA (US); Sarah Katherine Baxter, Seattle, WA (US)

(72) Inventors: Barry L. Stoddard, Bellevue, WA (US); Abigail Rose Lambert, Kenmore, WA (US); Ryo Takeuchi, Seattle, WA (US); Andrew M. Scharenberg, Seattle, WA (US); Sarah Katherine Baxter, Seattle, WA (US)

(21) Appl. No.: 13/702,972

(22) PCT Filed: Jun. 7, 2011
(86) PCT No.: PCT/US11/39527

§ 371 Date: Jan. 15, 2014

Related U.S. Application Data
(60) Provisional application No. 61/352,319, filed on Jun. 7, 2010.

(51) Int. Cl. C12N 15/10

(52) U.S. Cl. CPC C12N 15/1037
USPC 506/11

(57) ABSTRACT

The present disclosure provides compositions and methods for producing and expressing variant or engineered I-OnuI endonucleases, variant or engineered I-OnuI homologues, and hybrids of two I-OnuI or I-OnuI homologue domains that have a target site altered from the wild-type. A method for selecting a variant or engineered I-OnuI endonuclease, I-OnuI endonuclease homologue, and a hybrid of two I-OnuI or I-OnuI homologue domains that have a target site altered from the wild-type and directed to a site within a gene of interest is also provided. In addition, the present disclosure provides the crystal structure of the I-OnuI and I-LtrI endonucleases; the specificity profiles for both endonuclease for DNA binding and cleavage; the identity of amino acid residue positions in the I-OnuI and I-LtrI protein scaffold that determine DNA recognition specificity; methods for determining amino acid substitutions at those positions that alter DNA cleavage specificity; methods for the complete redesign of the DNA cleavage specificity of I-OnuI and its homologues for recognition and cleavage of a human gene of interest; and the relationship of the amino acid sequence, structure and specificity of I-OnuI to a collection of identifiable I-OnuI endonuclease homologues.

### CROSS-REFERENCE TO RELATED APPLICATIONS

This application claims priority to U.S. Provisional Application No. 61/352,319, filed Jun. 7, 2010, the disclosure of which is incorporated herein by reference.

### STATEMENT AS TO RIGHTS TO INVENTIONS MADE UNDER FEDERALLY SPONSORED RESEARCH AND DEVELOPMENT

The present invention was developed in part with government support under grant numbers R01 GM49857, RL1 CA133833, RL1 CA133832 and UL1 DE019582 awarded by the National Institutes of Health. The Government has certain rights in this invention.

### STATEMENT REGARDING SEQUENCE LISTING

The sequence listing associated with this application is provided in text format in lieu of a paper copy and is hereby incorporated by reference into the specification. The name of the text file containing the sequence listing is: 37147_SEQ_FINAL.txt. The file is 153 KB; was created on Jun. 7, 2011; and is being submitted via EFS-Web with the filing of the specification

### BACKGROUND

The term “Genome Engineering” describes an emerging discipline in which genomes of target organisms or cells are manipulated in vivo, generally using site specific recombination or other modifications to alter or add genetic information at specific chromosomal loci (through the targeted insertion, modification, or deletion of coding sequence). The concept of genome engineering dates back to experiments in the late 1970s in which ectopic DNA could be incorporated into the genome of the budding yeast Saccharomyces cerevisia. (Hinnen et al., Proc. Nat'l. Acad. Sci. USA 75:1929-1933, 1978; Orr-Weaver et al., Proc. Nat'l. Acad. Sci. USA 78:6354-6358, 1981). Depending on the exact methodology, individual yeast genes could be efficiently incorporated, deleted, mutated or corrected. However, while homologous recombination is extremely efficient in yeast, in mammalian cells it occurs at a very low frequency, often in the range of 10⁻⁵ to 10⁻⁷ per transformed cells. (Doestschman et al., Nature 330:576-578, 1987; Koller and Smithies, Proc. Nat'l. Acad. Sci. USA 86:8932-8935, 1989). As described below, this limitation can be in part overcome by using a highly site-specific endonuclease to cleave the donor or recipient locus to stimulate targeted recombination at a single chromosomal target site. The development of these reagents has allowed the field of genome engineering to progress dramatically over the past five years together with the pursuit of several specific applications. (Porteus, Mol. Ther. 13:438-446, 2006; Paques and Duchateau, Curr. Gene Ther. 7:49-66, 2007; Grizot et al., Nucl. Acids Res. 37:5405-5419, 2009).

The use of a site-specific endonuclease or a recombinase to embed synthetic genes at specific desired target sites in model organisms represents a crucial enabling technology for synthetic biologists to create, manipulate, and control artificial genomes. (Collins et al., Nucl. Acids Res. 38:2513, 2010). In particular, the ability to manipulate plant genomes in a controlled manner using targeted recombination is of great importance for the development of agricultural crop species both for food and for biofuel applications. (Porteus, Nature 459:337-338, 2009).

Synthetic genes encoding artificial site specific endonucleases can be used to create “selfish” genetic elements with the ability to integrate into and alter target genes while promoting their own transmission. This strategy has been proposed as a novel means for genetic control of Anopheles-mediated malaria transmission by dominant transmission and inheritance of traits corresponding to resistance against Plasmodium infection, or by reducing the lifespan or reproductive fitness of the insect host. (Chase, Plant Sci. 11:7-9, 2006).

Curative treatments for genetic diseases remain a critical area for research. (Ratjen and Dorign, Lancet 361:681-689, 2003; Griesenbach et al., Gene Ther. 13:1061-1067, 2006). Gene therapy approaches that rely on “gene augmentation” (i.e., ‘traditional’ gene therapy, where a wild-type gene is integrated into a patient's somatic genome) are under active investigation. Many early problems associated with this area (poor gene delivery, immune reactions to viral delivery vehicles, and oncogenesis) are being addressed. (Griesenbach et al., Gene Ther. 13:1061-1067, 2006; Verma and Weitzman, Annu. Rev. Biochem. 74:711-738, 2005). However, the current practice of gene replacement therapy still has several attendant issues: First, gene therapy involves the random insertion of foreign DNA into the genomes of stem cells, potentially resulting in the inactivation or activation of endogenous genes. (Abbott, Nat. Med. 12:597, 2006; Hacien-Bey-Abina et al., Science 302:415-419, 2003; Themis et al., Mol. Ther. 12:763-771, 2005). New lentiviral vectors avoid the use of highly active LTR-based promoters, and thus may improve safety profiles. (Griesenbach et al., Gene Ther. 13:1061-1067, 2006).

A second issue for present gene replacement therapies is that it is desirable to use lineage-specific transcriptional control elements. However, defining such control elements is non-trivial, and may require years of experimentation. (Puthenveetil et al., Blood 104:3445-3453, 2004; Malik and Arumugam, Hematology Am. Soc. Hematol. Edu. Program 45-50, 2005; Malik et al., Ann. NY Acad. Sci. 1054:238-249, 2005). A third issue is that traditional gene therapy is poorly suited for diseases caused by the presence of an aberrantly functioning protein that may interfere with function of the normal ‘replacement’ protein. Finally, the problem of maintaining long-term protein expression after treatment remains problematic due to epigenetic silencing after gene integration.

Therefore, targeted “gene repair” or “gene correction” strategies, using highly specific endonucleases to stimulate homologous recombination and endogenous gene repair at a desired genomic target site, have been proposed. (Porteus, Mol. Ther. 13:438-446, 2006; Paques and Duchateau, Curr. Gene Ther. 7:49-66, 2007). While gene repair has the same goal as traditional gene therapy approaches—restoration of the expression of a normally functioning protein—it has many advantages. Since the endogenous gene's function is restored, the protein is expressed under the control of its natural regulatory elements, thus eliminating potential problems with inappropriate or inadequate expression of a transgene or transgene silencing. By targeting the repair with high efficiency to a single mutant locus, gene repair may also be able to dramatically reduce mutagenesis due to random insertions at other genomic locations.

Several different technologies have been developed to promote efficient targeted genetic modification. These include gene-targeted triplex forming oligonucleotides and hybrid RNA-DNA oligonucleotides (Kolb et al., Trends Biotechnol. 23:399-406, 2005) and the use of highly site-specific recombinases and transposases (Coates et al., Trends Biotechnol. 23:407-419, 2005). Each of these approaches has limitations related to the range of sequences that can be targeted (e.g., triplex-forming oligonucleotides), or the requirement for prior introduction of a target site (e.g., for recombinase-mediated targeting).

Potentially the most versatile of all genome engineering technologies are those that make use of DNA double strand break-targeted homologous recombination for gene modification (FIG. 1). This method allows a desired genomic sequence to be altered in a precise manner, without the requirement for a selection marker or the introduction of additional exogenous DNA sequence(s). Double strand break-targeted recombination requires the introduction or expression of a site-specific endonuclease in cells to generate a DNA double strand break at or near the desired modification site, together with the presence of a DNA repair template. Repair templates typically flank the DNA double strand break site and include sequence modifications to be incorporated upon repair.

A significant practical barrier to widespread application of this accurate and efficient gene repair mechanism in genome engineering has been the requirement for an endonuclease that is able to induce DNA double strand breaks at specific chromosomal target sites. Over the past several years, two different approaches to creating enzymes capable of inducing highly site-specific DNA double strand breaks have been developed: zinc finger nucleases (ZFNs) and homing endonucleases (HEs).

A zinc finger nuclease is comprised of a non-specific nuclease domain (such as the catalytic domain of the FokI restriction endonuclease) tethered to a DNA-recognition and binding construct consisting of a tandem array of zinc fingers. (Porteus, Mol. Ther. 13:438-446, 2006; Smith et al., Nucl. Acids Res. 28:3361-3369, 2000; Bibikova et al., Mol. Cell. Biol. 21:286-297, 2001). As individual zinc fingers recognize DNA triplets within the context of long cognate target sites (Beerli and Barbas, Nature Biotechnol. 20:130-141, 2002; Bulyk et al., Proc. Nat'l. Acad. Sci. USA 98:7158-7163, 2001; Segal et al., Proc. Nat'l. Acd. Sci. USA 96:2758-2763, 1999), the concatenation of a series of zinc fingers of defined triplet specificity provides the possibility to create ZFNs able to bind and cleave at rare DNA targets. ZFNs have been demonstrated to induce gene correction in both Drosophila and mammalian cells (Bibikova et al., Science 300:764, 2003; Porteus and Baltimore, Science 300:763, 2003), and the highly efficient correction of disease-associated mutations in the human IL2Rγ gene (Urnov et al., Nature 435:646-651, 2005). Zinc finger nucleases have the important advantage of some capacity for modular design, and therefore ZFN technology has been the subject of intensive study over the past ten years. (Porteus, Mol. Ther. 13:438-446, 2006).

Homing is the process by which mobile microbial intervening genetic sequences—group I or group II introns or inteins—are duplicated into host genes that lack such a sequence. (Dujon, Gene 82:91-114, 1989; Lambowitz and Belfort, Annu. Rev. Biochem. 62:587-622, 1993; Belfort and Perlman, J. Biol. Chem. 270: 30237-30240, 1995; Belfort and Roberts, Nucl. Acids Res. 25:3379-3388, 1997; Chevalier and Stoddard, Nucl. Acids Res. 29:3757-3774, 2001). This process is induced by a site-specific homing endonuclease encoded by an open reading frame (ORF) that is harbored within the intervening sequence. (Jacquier and Dujon, Cell 41:383-394, 1985). The endonuclease specifically recognizes a target sequence corresponding to the intron insertion site and generates a single- or double-strand break that is repaired by cellular machinery. If the intron-containing allele is used as a template for repair via homologous recombination, the intron and its resident endonuclease gene is duplicated into the target site and the homing cycle is completed. Transfer of mobile introns can be extremely efficient, leading to unidirectional gene conversion events in diploid genomes (Jacquier and Dujon, Cell 41:383-394, 1985), genetic competition in mixed phage infections (Goodrich-Blair and Shub, Cell 84:211-221, 1996), gene transfer between different subcellular compartments of unrelated organisms (Turmel et al., Mol. Biol. Evolution 12:533-545, 1995), and rapid genetic spread (Cho et al., Proc. Nat'l. Acad. Sci. USA 95:14244-14249, 1998).

Homing endonucleases are widespread and found within introns and inteins in all biological super-kingdoms. At least six homing enzyme families have been identified (FIG. 2); each is associated with a unique host genome. The LAGLIDADG endonuclease (LHE) are found in archaea, fungi and algae, the His-Cys Box family is found in protists, the HNH, GIY-YIG and VSR-like endonucleases (all found primarily in bacteriophage) and the PD-(D/E)xK family are found in bacteria.

In order to promote precise intron transfer and avoid deleterious cleavage of their host genomes, homing endonucleases are highly sequence-specific. However, they exhibit sufficient site recognition flexibility to promote genetic mobility in the face of target site variation across diverging host strains. Homing endonucleases use a strategy in which variable numbers of contacts are made to individual base pairs across a long target site (providing overall high specificity combined with variable recognition fidelity across the site). The individual polymorphisms that are tolerated by the enzyme are strongly correlated with the conservation of the sequence of the host target site. For a LAGLIDADG homing endonuclease (LHE), the specificity of DNA recognition is at least 1 in 10⁹. (Chevalier et al., J. Mol. Biol. 329:253-269, 2003). By altering the DNA cleavage specificity of homing endonucleases in the laboratory, a wide variety of such enzymes can be generated for genome engineering applications. (Paques and Duchateau, Curr. Gene Ther. 7:49-66, 2007). Several strategies have been investigated including: a) domain shuffling and fusions wherein domains from unrelated free-standing LHEs can be structurally fused to create chimeric homing endonucleases that recognize corresponding chimeric target sites. (Chevalier et al., Molec. Cell. 10:895-905, 2002). In addition, monomeric endonucleases can also be created from homodimeric proteins. (Li et al., Nucl. Acids Res. 37:1650-1662, 2009); b) base-pair specificity changes using selections, screens and redesigns which include several methods that focus on mutation of endonuclease side chains that contact individual DNA basepairs to alter specificity. These methods include: i) selections for efficient cleavage activity (Seligman et al., Nucl. Acids Res. 30:3870-3879, 2002; Gruen et al., Nucl. Acids Res. 30:e29, 2002, Sussman et al., J. Mol. Biol. 342:31-41, 2004; Rosen et al., Nucl. Acids Res. 34:4791-4800, 2006) or cleavage-induced homologous recombination events (Arnould et al., J. Mol. Biol. 355:443-458, 2006; Chames et al., Nucl. Acids Res. 33:e178, 2005); ii.) structure-based computational redesign of DNA-contact surfaces and residues, to alter homing endonuclease-DNA contacts (Ashworth et al., Nature 441:656-659, 2006; Ashworth et al., Nucl. Acids Res. 38:5601-5608, 2010) or to facilitate efficient mutational screening of enzyme libraries (Sussman et al., J. Mol. Biol. 342:31-41, 2004, Arnould et al., J. Mol. Biol. 355:443-458, 2006; Chames et al., Nucl. Acids Res. 33:e178, 2005); iii.) surface display using either B-cells (Volna et al., Nucl. Acid Res. 35:2748-2758, 2007) or yeast (Jarjour et al., Nucl. Acids Res. 37:6871-6880, 2009) to facilitate characterization of binding and cleavage specificity profiles, and to sort populations of mutated endonucleases for shifts in binding specificity.

### SUMMARY

The present disclosure provides compositions and methods for producing and expressing variant or engineered I-OnuI endonuclease, variant or engineered I-OnuI homologues, and hybrids of two I-OnuI or I-OnuI homologue domains that have a target site altered from the wild-type. A method for selecting a variant or engineered I-OnuI endonuclease, I-OnuI homologue, and a hybrid of two I-OnuI or I-OnuI homologue domains with a target site modification from the wild-type and directed to a site within a gene of interest are also provided. The method for selecting a variant or engineered I-OnuI endonuclease comprises the steps of: i) determining the target site for an I-OnuI endonuclease; ii) searching a nucleic acid database for a gene of interest comprising a nucleotide sequence that is at least 40% identical to the nucleotide sequence of the target site of the I-OnuI endonuclease; iii) selecting a gene of interest comprising the nucleotide sequence that is at least 40% identical to the nucleotide sequence of the target site of the I-OnuI endonuclease; iv) constructing a molecular model of the I-OnuI endonuclease bound to the nucleotide sequence that is at least 40% identical to the nucleotide sequence of the target site of the I-OnuI endonuclease from the gene of interest; v) mutating the I-OnuI endonuclease at amino acid residues that have been determined to be direct contact residues, backbone contact residues, or water-mediated contact residues with the target site of the gene of interest to form a library of variant or engineered I-OnuI endonuclease; vi) expressing the library of variant or engineered I-OnuI endonuclease; vii) screening the library of variant or engineered I-OnuI endonuclease for binding activity to the target sequence in the selected gene and the cleavage activity for the target sequence in the selected gene; and viii) selecting an altered, variant or engineered, I-OnuI endonuclease that can act upon a nucleotide sequence containing a modification in the target site from the wild-type and directed to the target site within the gene of interest, wherein the binding and cleavage activity is highest for the target sequence in the gene of interest.

The method for selecting a variant or engineered I-OnuI endonuclease homologue with a target site modification from the wild-type and directed to a site within a gene of interest comprises the steps of: i) determining the target site for a I-OnuI endonuclease homologue; ii) searching a nucleic acid database for a gene of interest comprising a nucleotide sequence that is at least 40% identical to the nucleotide sequence of the target site of the I-OnuI endonuclease homologue; iii) selecting a gene of interest comprising the nucleotide sequence that is at least 40% identical to the nucleotide sequence of the target site of the I-OnuI endonuclease homologue; iv) constructing a molecular model of the I-OnuI endonuclease homologue bound to the nucleotide sequence from the gene of interest that is at least 40% identical to the nucleotide sequence of the target site of the I-OnuI endonuclease homologue; v) mutating the I-OnuI endonuclease homologue at amino acid residues that have been determined to be direct contact residues, backbone contact residues, or water-mediated contact residues with the target site of the gene of interest to form a library of variant or engineered I-OnuI endonuclease homologues; vi) expressing the library of variant or engineered I-OnuI endonuclease homologues; vii) screening the library of variant or engineered I-OnuI endonuclease homologues for binding activity to the target sequence in the selected gene and the cleavage activity for the target sequence in the selected gene; and viii) selecting the variant or engineered I-OnuI endonuclease homologue that can act upon the modification in the target site from the wild-type and directed to the target site within the gene of interest, wherein the binding and cleavage activity is highest for the target sequence in the gene of interest.

The method for selecting an engineered hybrid of two I-OnuI or I-OnuI homologue domains with a target site modification from the wild-type and directed to a site within a gene of interest comprises the steps of: i) determining the target site for a hybrid of two I-OnuI or I-OnuI homologue domains; ii) searching a nucleic acid database for a gene of interest comprising a nucleotide sequence that is at least 40% identical to the nucleotide sequence of target site of the hybrid of two I-OnuI or I-OnuI homologue domains; iii) selecting a gene of interest comprising the nucleotide sequence that is at least 40% identical to the nucleotide sequence of the target site of the hybrid of I-OnuI or I-OnuI homologue domains; iv) constructing a molecular model of the hybrid of two I-OnuI or I-OnuI homologue domains bound to the nucleotide sequence that is at least 40% identical to the nucleotide sequence of the target site of the hybrid of two I-OnuI or I-OnuI homologue domains from the gene of interest; v) mutating the hybrid of two I-OnuI or I-OnuI homologue domains at amino acid residues that have been determined to be direct contact residues, backbone contact residues, or water-mediated contact residues with the target site of the gene of interest to form a library of variant or engineered hybrids of two I-OnuI or I-OnuI homologue domains; vi) expressing the library of variant or engineered hybrids of I-OnuI or I-OnuI homologue domains; vii) screening the library of variant or engineered hybrids of I-OnuI or I-OnuI homologue domains for binding activity to the target sequence in the selected gene of interest and the cleavage activity for the target sequence in the selected gene of interest; and viii) selecting the engineered hybrid of two I-OnuI or I-OnuI homologue domains that can act upon the nucleotide sequence containing the modification in the target site from the wild-type and directed to a target site within the gene of interest, wherein the binding and cleavage activity is highest for the target sequence in the gene of interest.

Methods are also provided for producing an engineered endonuclease that can bind and cleave a specific site within a gene of interest. In this method a library of target sites for various endonuclease related to I-OnuI is established and the library is searched for a target site that has about 40% sequence identity with a nucleic acid sequence within the gene of interest. The selected endonuclease, homologue or hybrid can then be engineered. A method for producing an engineered I-OnuI endonuclease, an engineered I-OnuI endonuclease homologue, or a hybrid of two I-OnuI or I-OnuI homologue domains with a target site modification from the wild-type and directed to a site within a gene of interest comprises the steps of: i) determining the nucleotide sequence of the gene of interest; ii) searching a nucleic acid database comprising the target sites for I-OnuI endonuclease, I-OnuI endonuclease homologues, and hybrids of two I-OnuI or I-OnuI homologue domains for a I-OnuI endonuclease, I-OnuI endonuclease homologues, and hybrids of two I-OnuI or I-OnuI homologue domains comprising a nucleotide sequence that is at least 40% identical to a nucleotide sequence of within the gene of interest; iii) selecting the I-OnuI endonuclease, I-OnuI endonuclease homologues, and hybrids of two I-OnuI or I-OnuI homologue domains comprising the I-OnuI endonuclease, I-OnuI endonuclease homologues, and hybrids of two I-OnuI or I-OnuI homologue domains with the target site that is at least 40% identical to the nucleotide sequence within the gene of interest; iv) constructing a molecular model of the selected I-OnuI endonuclease, I-OnuI endonuclease homologues, and hybrids of two I-OnuI or I-OnuI homologue domains bound to the target site that is at least 40% identical to the nucleotide sequence within the gene of interest with the nucleic acid sequence within the gene of interest; v) mutating the selected I-OnuI endonuclease, I-OnuI endonuclease homologues, and hybrids of two I-OnuI or I-OnuI homologue domains at amino acid residues that have been determined to be direct contact residues, backbone contact residues, or water-mediated contact residues with the target site of the gene of interest to form a library of variants or engineered I-OnuI endonuclease, variant or engineered I-OnuI endonuclease homologues, and hybrids of two I-OnuI or I-OnuI homologue domains; vi) expressing the library of variant or engineered I-OnuI endonuclease, I-OnuI endonuclease homologues, and hybrids of two I-OnuI or I-OnuI homologue domains; vii) screening the library of variant or engineered I-OnuI endonuclease, I-OnuI endonuclease homologues, and hybrids of two I-OnuI or I-OnuI homologue domains for binding activity to the target sequence in the gene of interest and the cleavage activity for the target sequence in the gene of interest; and viii) selecting the variant or engineered I-OnuI endonuclease, I-OnuI endonuclease homologues, and hybrids of two I-OnuI or I-OnuI homologue domains that can act upon a nucleotide sequence containing a modification in the target site from the wild-type and directed to a target site within the gene of interest, wherein the binding and cleavage activity is highest for the target sequence in the gene of interest.

The present disclosure also describes variant or engineered I-OnuI endonuclease, I-OnuI endonuclease homologues and variants thereof, and hybrids of two I-OnuI or I-OnuI homologue domain polypeptides, vectors comprising nucleic acid sequence that express variant or engineered I-OnuI endonuclease, I-OnuI endonuclease homologues and variants thereof, and hybrids of two I-OnuI or I-OnuI homologue domain polypeptides, host cells, and various methods for the use of the variant or engineered I-OnuI endonuclease, I-OnuI endonuclease homologues and variants thereof, and hybrids of two I-OnuI or I-OnuI homologue domains.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 depicts gene modification using a site-specific endonuclease. A double-strand break induced at or near a targeted chromosomal sequence (‘X’ on each strand) stimulates subsequent homologous recombination (HR), using an exogenous DNA template (typically provided by a plasmid transfection or a viral vector) as the donor of homologous DNA sequence. HR can result in targeted gene insertion, deletion, modification or mutation. In the absence of the donor DNA template, cleavage by the homing endonuclease can result in mutation of the target site as a result of nonconservative repair via DNA end-joining.

FIG. 2 depicts the known structural families of homing endonucleases. Each protein fold shown above is named on the basis of conserved sequence motifs, and is largely restricted to individual biological hosts and corresponding genomes.

FIGS. 3A and B depict the expression and purification of I-OnuI. FIG. 3A: The homing endonuclease was expressed as a fusion with an N-terminal glutathione-S-transferase, purified by affinity chromatography and liberated by proteolytic processing with a protease designed for site specific cleavage of the GST tag (PreScission® protease; GE Healthcare). The protein was concentrated to 200 micromolar concentration in 50 mM Hepes-NaOH (pH 7.5), 150 mM NaCl, 20 mM MgCl₂, 5% glycerol. FIG. 3B: I-OnuI binds its naturally occurring target site (corresponding to the intron insertion site in the RP3 gene in its biological host with a dissociation constant (K_{d}) of approximately 37 picomolar, as determined by electrophoretic mobility shift analysis (EMSA: gel shift). The cleavage activity of the enzyme at a similar apparent enzyme concentration was confirmed using the same substrate sequence in a reporter plasmid (bottom).

FIG. 4 provides the X-ray crystal structure of the I-OnuI homing endonuclease bound to its DNA target site. The structure was solved and refined at 2.4 angstrom resolution. The X-ray data and refinement statistics are provided in the table (right). The structure of the protein-DNA complex is displayed to the left in two separate orientations. The white circles highlight the ‘LAGLIDADG’ helices at the center of the interface between the two endonuclease domains.

FIG. 5 depicts the DNA-protein interface of I-OnuI. The 22 basepair target sequence (SEQ ID NO: 1 and SEQ ID NO: 2) is contacted by a combination of direct and water-mediated contacts, both to the DNA bases and its phosphoribosyl backbone. The scissile phosphates are white; the ‘central four’ basepairs between those phosphates are dark grey. Waters are black spheres; bound divalent metal ions are light gray spheres.

FIGS. 6A and 6B provide the specificity profile of I-OnuI, determined using I-OnuI displayed on a yeast surface. FIG. 6A demonstrates the relative ability of DNA sequences harboring individual basepair mismatches to be cleaved (the wild-type base at each position gives equivalent signal in the assay; bars with reduced heights indicate reduced cleavage; bars with elevated heights (for example, −10A) indicate improved cleavage. The wild-type target sequence (SEQ ID NO: 1) is depicted at the top of the figure. FIG. 6B indicates the ability of the same target site variants to be recognized and bound by the enzyme. Those basepairs that can be bound and cleaved, in these separate experiments, as well or nearly as well as wild-type enzyme include −6C, −4C, +4T, and +4C.

FIG. 7 depicts the designed and/or selected mutations in the I-OnuI enzyme scaffold that correspond to altered DNA cleavage specificity at individual basepair positions in the endonuclease target site. The positions and identity of the basepair substitutions are indicated in the left most column and by the base listed in bold in the target sites. SEQ ID NO: 3 through SEQ ID NO: 33. The corresponding mutations in the I-OnuI scaffold are indicated in the right column. Those that are shown in bold correspond to computationally designed DNA-contacting side chains (created using the crystal structure as a guide); those that are shown in italics correspond to mutations generated by selection experiments using an in vivo screen for cleavage and elimination of a reporter gene.

FIG. 8 is an illustration of a human gene target (monoamine oxidase B) for targeted gene modification by a redesigned I-OnuI scaffold. The chromosomal locus and gene organization of MAO-B, a comparison of the nucleotide sequence of the target site (SEQ ID NO: 34) within MAO-B to be used for engineering a I-OnuI variant and the nucleotide sequence of the wild-type target site of I-OnuI (SEQ ID NO: 1) is also provided.

FIG. 9 shows a summary of the selection of mutated variants of I-OnuI that display cleavage activity towards the MAO-B gene target (SEQ ID NO: 34). Mutation and selection of I-OnuI (SEQ ID NO: 35 through SEQ ID NO: 51) was conducted using an iterative approach in which individual basepair variants in the target site were incorporated in an iterative manner. Of the final “Round 3” constructs, R3 #3 (SEQ ID NO: 48), R3 #6 (SEQ ID NO: 50 and R3 #8 (SEQ ID NO: 51) displayed the highest cleavage activity towards the MAO-B target; one (R3 #3) was chosen for full characterization.

FIGS. 10A through 10C show the altered DNA cleavage specificity of the redesigned and selected “R3 #3” variant of I-OnuI. FIG. 10A: titrations of wild-type (WT) and redesigned (R3 #3) I-OnuI against WT target and MAO-B target. FIG. 10B: Plots of cleavage progression against the WT and MAO-B DNA target sequences. FIG. 10C: K_{d} and relative cleavage of wild-type and engineered variant of I-OnuI towards its target sites. Binding affinities were determined by electrophoretic gel shift analyses; k_{cat}/Kₘ by traditional endonuclease cleavage assays using radiolabeled oligonucleotide substrates. Non-cognate site cleavage by the wild-type and engineered versions of I-OnuI was undetectable; estimated detection limits for the assay are provided.

FIG. 11 provides the protein sequence of the I-OnuI LAGLIDADG homing endonuclease and its immediate homologues (40% or higher sequence identity). All of these enzymes are encoded within algal and fungal organellar genomes, and are found in a wide variety of host genes (including those encoding several ribosomal proteins, cytochrome oxidases, ubiquinone oxidoreductase subunits, and ATP synthase subunits).

FIGS. 12A and 12B depict the crystal structure of I-LtrI and provides a schematic of its DNA binding surface. The crystal structure of I-LtrI bound to its DNA target sequence (SEQ ID NO: 86 and SEQ ID NO: 87) is shown in two different orientations and was solved to 2.7 Å resolution. Superposition of the structure on that of I-OnuI yields an RMSD across 284 superimposed a-carbons of approximately 1.3 Å and a similar DNA backbone configuration. The two −DNA interface of I-LtrI was quite dissimilar to that of I-OnuI. Only one side chain-nucleotide contact (between glutamine 195 and the adenine ring at base pair position +9) was observed in both structures. Taken together, these results suggest that even closely related LHEs such as I-OnuI and I-LtrI rapidly evolve unique, diverged surfaces for recognition of corresponding DNA target sites, while maintaining conserved protein folds and catalytic mechanisms.

FIGS. 13A through C depict the expression and cleavage activity of I-OnuI, I-LtrI and I-GpiI on yeast cell surface. Cells were stained with an anti-Myc probe (to visualize folding and surface expression; horizontal axis) and for binding of a DNA duplex containing the validated I-OnuI (FIG. 13A; SEQ ID NO: 88 and SEQ ID NO: 89) and I-LtrI (FIG. 13B; SEQ ID NO: 90 and SEQ ID NO: 91) target sites or the predicted I-GpiI (FIG. 13C; SEQ ID NO: 92 and SEQ ID NO: 93) DNA target site (vertical axis). Cleavage activity was visualized by loss of bound labeled DNA from the yeast cell surface in the presence of magnesium (which facilitates cleavage activity; dark gray population in each plot) relative to staining in the presence of calcium (which inhibited cleavage; lighter gray population in each plot).

FIG. 14 demonstrates putative and validated DNA target sites for homologues of I-OnuI (see FIG. 3). Target sites typically correspond to exon boundaries at the site of the mobile intron/homing endonuclease gene insertion in the host organism and genome (5′ and 3′ exons of the individual host genes are shown underlined and double underlined, respectively). Four endonucleases from this list (I-OnuI, I-LtrI, I-GpiI and I-MpeI) have been purified and tested against putative target sites, leading to validation of their DNA cleavage activities.

FIGS. 15A through D demonstrate the target sites and catalytic activity for a series of homologues of I-OnuI. FIG. 15A depicts the target sites for I-OnuI (SEQ ID NO: 1), I-GpeI (SEQ ID NO: 94); I-LtrI (SEQ ID NO: 86); I-GpiI (SEQ ID NO: 95); I-MpeI (SEQ ID NO: 96); I-PanII (SEQ ID NO: 97); I-GzeI (SEQ ID NO: 98); I-SscI (SEQ ID NO: 99); I-AabI (SEQ ID NO: 100); I-PnoI (SEQ ID NO: 101); I-GzeII (SEQ ID NO: 102); I-CpaIII (SEQ ID NO: 103); I-LtrII (SEQ ID NO: 104); and I-SmaI (SEQ ID NO: 105). The cleavage of the predicted target sites were assayed using three different methods. FIG. 15B depicts the results from a flow-cytometry based tethered DNA cleavage assay for I-GzeII. The flow cytometry-based assay used surface-expressed enzyme to cleave a fluorescently-tagged DNA substrate. Cleavage of the DNA sequence was visualized by a shift in the fluorescent signal. FIG. 15C depicts the sequencing of the cleavage digest using a plasmid substrate. The target sequence for the enzyme I-GpiI (SEQ ID NO: 106 and SEQ ID NO: 107) was cloned into a circular plasmid and the plasmid substrate was digested by the enzyme. The resulting linearized DNA was sequenced from both sides of the break to determine the precise cleavage position on each strand of the DNA. This allowed for identification of the exact center of each target site. FIG. 15D depicts an in vitro cleavage digest for I-AabI. The surface-displayed enzyme was released from the yeast surface and used directly for an in vitro digest of the labeled DNA substrate. The cleaved oligonucleotide was distinguishable when visualized on an acrylamide gel.

FIG. 16 depicts homology models of two I-OnuI homologues I-GpiI and I-MpeI. the models were based on the crystal structure of I-OnuI. Sequence alignments for these endonucleases and others in the I-OnuI family are shown in FIG. 11. Homology models of these and the other enzymes from FIG. 11 have been used to engineer or select enzyme variants with altered surfaces and/or DNA contact residues for the purpose of altering their solution behavior, stability, and/or DNA binding and cleavage specificities (as illustrated in FIGS. 17 and 18)

FIGS. 17A through 17F depict the transfer of amino acid residues between the surfaces of I-OnuI-family homologues to alter protein solution behavior, folding, and/or DNA recognition properties. In initial studies were designed to transfer or “graft” all unconstrained surface from one homologue to another in order to achieve higher DNA sequence identity between individual enzymes, and to alter the protein's solution behavior (FIGS. 17A through 17C). Approximately 40 to 60 mutations per homologue resulted in greater than 80% identity between the enzyme coding sequences, and excellent expression on the surface of yeast. DNA cleavage assays were performed to verify that activity was maintained throughout the engineering process. As an alternative, amino acid residues from the DNA-reaction surface can be grafted onto a different scaffold to successfully alter the DNA target specificity of that scaffold (FIGS. 17D through 17F). This approach can be used to create artificial enzymes using DNA-contacting amino acid residues transplanted between I-OnuI homologues. FIG. 17A depicts a surface representation of I-OnuI with the location of solvent-exposed mutations highlighted in black. FIG. 17B depicts the improvement in yeast surface expression of I-OnuI by incorporating surface amino acid mutations corresponding to polar amino acid residues transplanted from homologous homing endonucleases. The N-(APC) and C-(FITC) termini of the protein were fluorescently tagged to visualize stable full-length protein. Surface expression increased from 29% to 65%. FIG. 17C depicts the verification of the cleavage activity of the “resurfaced” variant of I-OnuI (xOnu3) using an in vitro cleavage assay. FIG. 17D provides a cartoon representation of the I-OnuI scaffold with positions of DNA-contacting residues and loops highlighted. These highlighted residues were transplanted from the I-MpeI endonuclease to the I-OnuI scaffold to create a different resurfaced variant termed “MpeItransOnuI”. FIG. 17E depicts the verification of stable surface expression for engineered MpeItransOnuI. FIG. 17F depicts the verification of cleavage activity forMpeItransOnuI using the I-MpeIDNA target in an in vitro cleavage assay. The ‘surface-transplanted’ endonuclease now recognized and cleaved the target site originally recognized by the I-MpeI endonuclease.

FIG. 18 shows the amino acid sequences of, and alignment between engineered variants of I-OnuI (“xOnuRound2”) (SEQ ID NO: 108) and I-LtrI (“xLtrRound2”) (SEQ ID NO: 109) that incorporate amino acid substitutions which increase sequence homology of each reading frame against one another (from 47% identity for the wild-type genes, to 76% for the redesigned endonucleases). The wild-type DNA cleavage specificities for these engineered variants were unchanged from wild-type scaffolds. This example demonstrates that hybrid, intermediate protein scaffold structures and sequences were fully accessible from the starting wild-type scaffolds in this protein family, and that further recombination and shuffling of these sequences can yield intermediate DNA cleavage specificities.

FIG. 19 shows that hybrid homing endonucleases can be constructed from fusions of N- and C-terminal structural domains from I-OnuI and/or its identifiable homologues. Chimeras of I-OnuI homologues are constructed by linking the N-terminal and C-terminal domains of two enzymes of interest (dark and light grey ribbon diagrams in the upper panel). Chimera assembly can be accomplished by gene synthesis, as well as by PCR gene-assembly of the respective halves, followed by restriction enzyme digestion and ligation of a shared site within the linker connecting the N- and C-terminal domains. The N-terminal domain is defined as the region approximately analogous to I-OnuI residues 1-162 (SEQ ID NO: 35), and the C-terminal domain is defined as the region approximately analogous to I-OnuI residues 163-303 (SEQ ID NO: 35). The exact location of N- and C-terminal division is variable, but lies within the 23 residues preceding the second LAGLIDADG helical region. Artificial linking residues can be included for ease of chimera construction, and for chimera optimization. Highly active chimera variants have been created using domains from I-OnuI and/or I-OnuI homologues I-GpiI, I-GzeI, I-SscI, I-PanII, and I-LtrI. The catalytic activity of constructed chimeras can be increased by randomization of protein residues within and near the LAGLIDADG helices and selection by flow-cytometry cleavage assays.

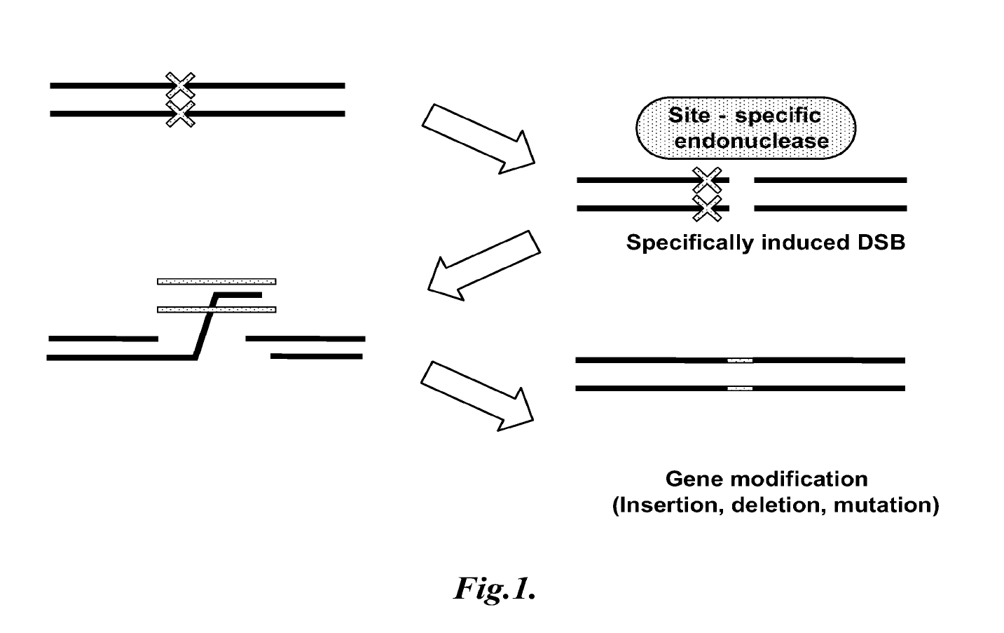

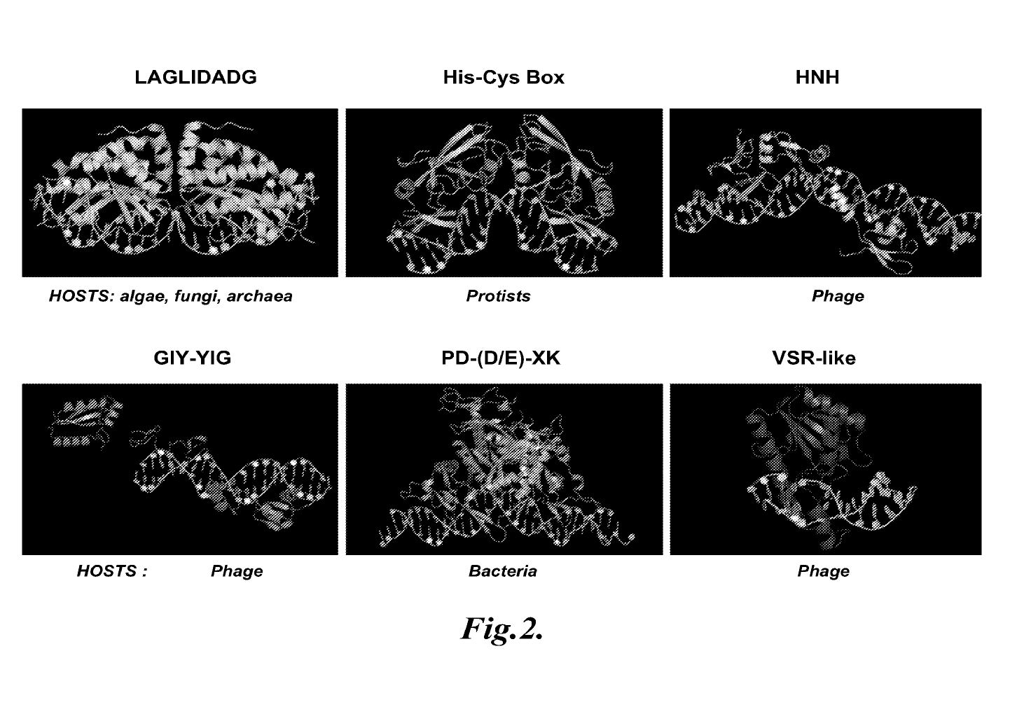

### DETAILED DESCRIPTION

Generally, the nomenclature used herein and many of the laboratory procedures in regard to cell culture, molecular genetics and nucleic acid chemistry, which are described below, are those well known and commonly employed in the art. (See generally Sambrook et al., Molecular Cloning: A Laboratory Manual, 3d Ed., Cold Spring Harbor Laboratory Press, New York (2001), which is incorporated by reference herein). Standard techniques are used for recombinant nucleic acid methods, site directed mutagenesis, preparation of biological samples, preparation of cDNA fragments, PCR, molecular modeling, crystallography, and the like. Generally enzymatic reactions and any purification and separation steps using a commercially prepared product are performed according to the manufacturers' specifications.

Homing endonucleases (HEs) are highly site-specific endonucleases that induce homologous recombination or gene conversion in vivo by cleaving long (typically greater than 20 basepair) DNA target sites. Homing endonucleases are under development as tools for applications that require targeted genome modification, including insertion, deletion, or modification of genetic coding sequences. The first structures of homing endonucleases were reported in 1997. Since that time, representative structures from each of the known families of homing endonuclease have been determined, and corresponding details of their mechanisms of DNA recognition and cleavage have been elucidated. Using this information, the LAGLIDADG homing endonuclease family (LHEs), which are distributed throughout single cell algal and fungi and in archaea, display the highest overall DNA recognition specificity. These proteins possess one or two LAGLIDADG catalytic motifs per protein chain and function as homodimers or as monomers, respectively. In addition, the family has been identified as the most tractable for further modification by structure-based selection and/or engineering approaches. To date, only the I-CreI-derived variants of the LAGLIDADG family have been used to modify endogenous chromosomal targets. Generation of these engineered I-CreI endonuclease was achieved by extensive alteration of the wild-type enzyme's DNA recognition specificity, at up to two-thirds of the base pair positions in a desired target site. The successful redesign of the I-CreI endonuclease has led to the development of enzymes that recognize and act at genes associated with monogenic diseases, including the human RAG1 and XPC genes (Redondo et al., Nature 456:107-113, 2008, Grizot et al., Nuc. Acids Res. 37:5405-5419, 2009), and the targeted disruption of at least one plant gene (LIGUELESS in corn) (Gao et al., Plant J. 61:176-187, 2010). These studies demonstrate the feasibility of using engineered homing endonucleases to promote efficient and target site-specific modification of chromosomal loci. While this particular enzyme has been exceptionally cooperative during the process of protein engineering, the reliance upon that single scaffold for genome engineering has limited the number of gene targets that can be modified using LHEs.

Use of many currently known homing endonucleases for the purpose of targeted gene insertion or modification is limited both by inappropriate biophysical behavior (for example, requiring thermophilic temperatures for DNA cleavage) or insufficient physiological cleavage activity, and by limitations on the extent which their DNA specificity can be altered. For any given enzyme, approximately one-third of the basepairs of its target site are not amenable to specificity-shifting redesign efforts. As well, extensive alteration of DNA binding specificity is often accompanied by losses of activity or affinity, or broadening of overall specificity, relative to the parental wild-type enzyme.

Therefore, there exists a need for the discovery, characterization, and engineering of a large collection of LAGLIDADG homing endonuclease scaffolds that possess the following characteristics: (1) monomeric (single chain) structures, (2) activity at physiological (30° to 37° C.) temperatures, (3) high solubility and stability at physiological pH and ionic strength, (4) high enough amino acid identity across a broad range of homologous protein scaffolds (at least about 40 to 50%) to allow the creation of chimeric, hybrid, shuffled and recombined enzymes with high specificity and activity, and (5) sufficient diversity in their DNA sequence recognition profiles to allow recognition and cleavage of a much wider range of genomic targets than is currently possible with an individual homing endonuclease such as I-CreI.

The present disclosure demonstrates that naturally occurring LHEs can exploited to rapidly create novel genome editing enzymes. In particular, the present disclosure focused on a single LHE subfamily (or ‘clade’) that are all related to the I-OnuI homing endonuclease, provides a surprisingly diverse set of DNA target site sequences that can be cleaved by these LHEs, that were otherwise closely related to one another. The target sites of these enzymes can be predicted and validated by analysis of the exon flanking sequences that surround the homing endonuclease genes in their natural host cells. The disclosure also provides the determined DNA-bound crystal structure of two representative enzymes from this enzyme subfamily in order to assess the conservation of their protein folds and DNA recognition mechanisms, and then created a variant of one of those enzymes in order to cleave and disrupt a predetermined human gene, the human monoamine oxidase B (MAO-B) gene. The present disclosure also demonstrates that hybrid enzymes can be created that contain distinct regions of multiple homing endonucleases, corresponding either to the transplantation and exchange of surface-exposed residues between enzyme homologues, or by fusion of unrelated N- and C-terminal domains between enzyme homologues. This demonstrates that systematic mining and characterization of sufficient numbers of naturally occurring LHE scaffolds can allow the full potential of these enzymes for routine gene targeting applications to be realized.

As such, disclosed herein are (a) the crystal structures of the I-OnuI and I-LtrI homing endonucleases; (b) the specificity profile of I-OnuI for DNA binding and cleavage; (c) the identity of amino acid residue positions in the I-OnuI and I-LtrI protein scaffolds that determine DNA recognition specificity; (d) examples of amino acid substitutions at those positions that alter DNA cleavage specificity; (e) an example of the complete redesign of the DNA cleavage specificity of I-OnuI for recognition and cleavage of a human gene therapy target (located in the monoamine oxidase-B gene target); (f) the relationship of the sequence, structure and specificity of I-OnuI to a collection of identifiable I-OnuI endonuclease homologues with a wide variety of known and predicted DNA specificities; (g) expression and characterization of identifiable homologues of I-OnuI; and (h) creation and expression of hybrid scaffolds containing combined sequence elements of individual wild-type homing endonuclease, at a level of homology appropriate for future recombination and shuffling experiments.

The endonuclease I-OnuI has been characterized by known methods to be a monomer displaying the characteristics of an LAGLIDADG homing endonuclease. As the molecule displayed certain characteristics required for gene targeting and subsequent engineerability the binding and cleavage activity of the isolated protein was determined. In addition, the crystal structure when bound to its substrate was determined. Further, homologues of I-OnuI were determined and are considered embodiments of the present disclosure that can be modified to alter their nucleic acid target sequence.

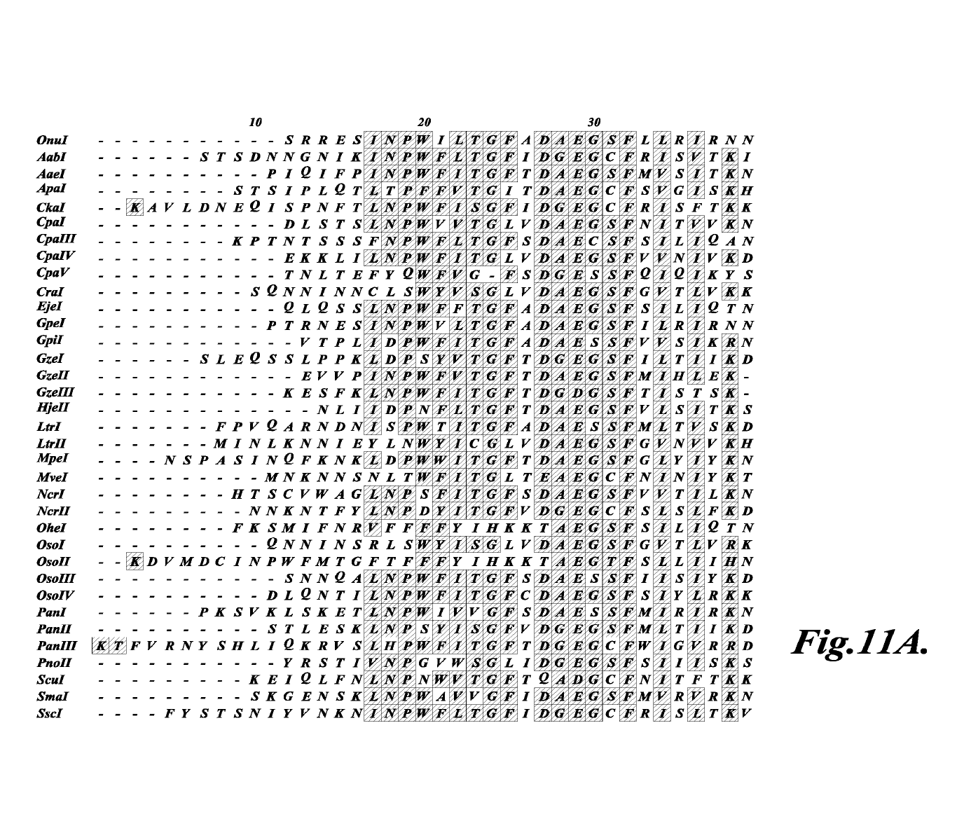

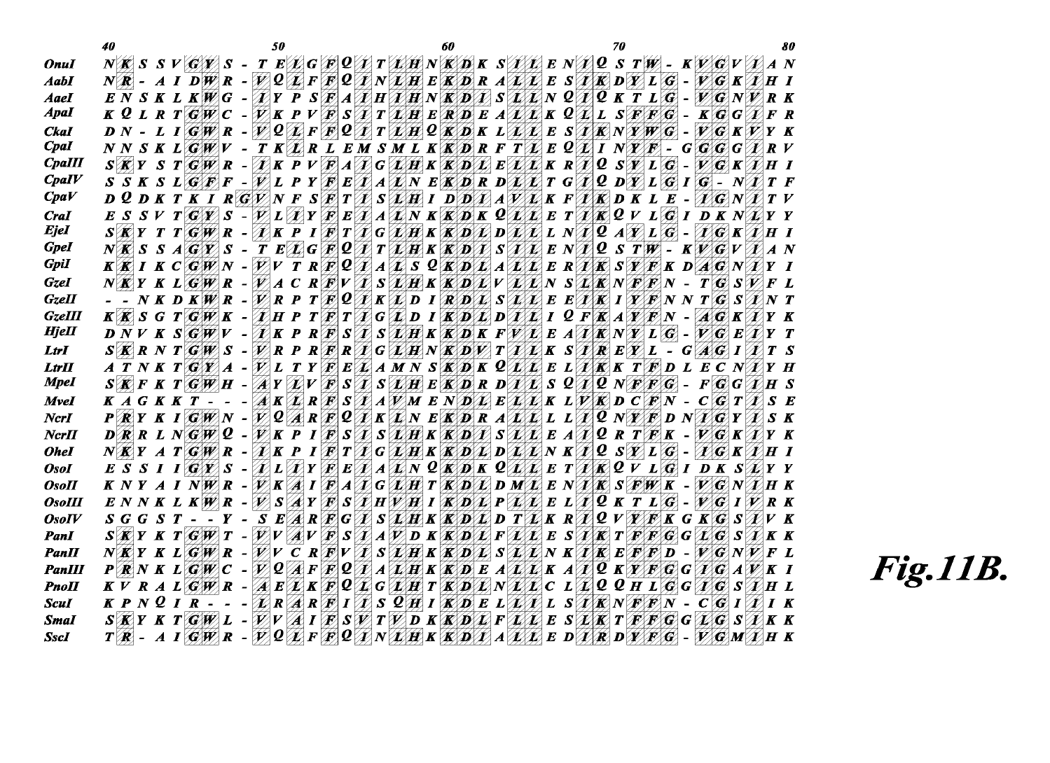

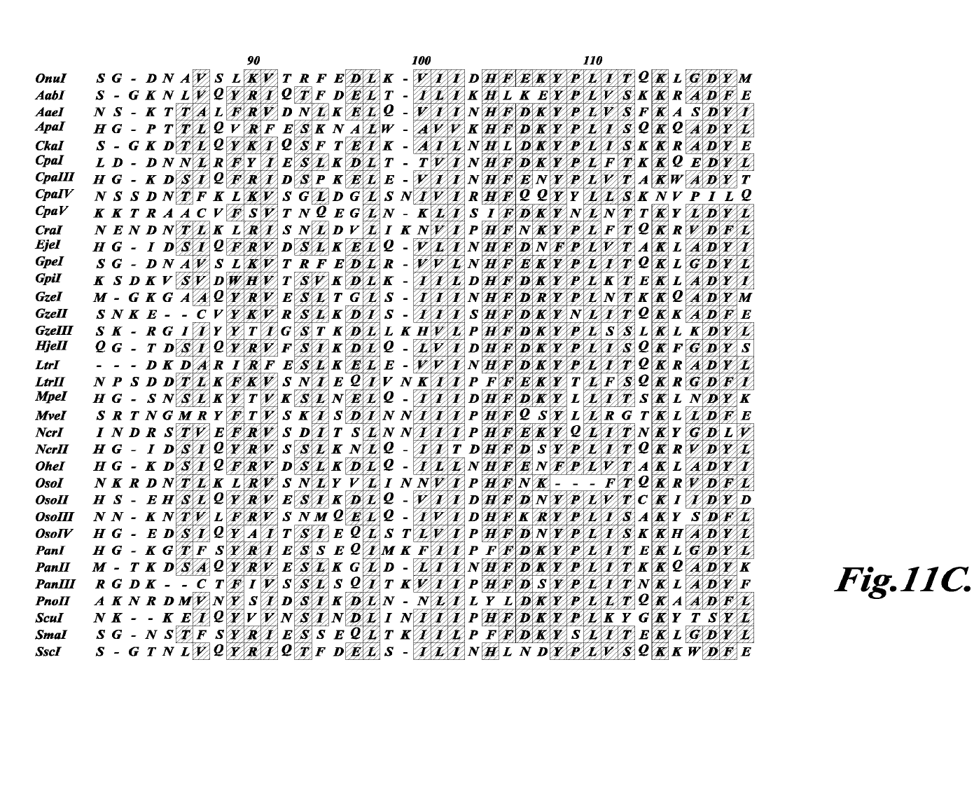

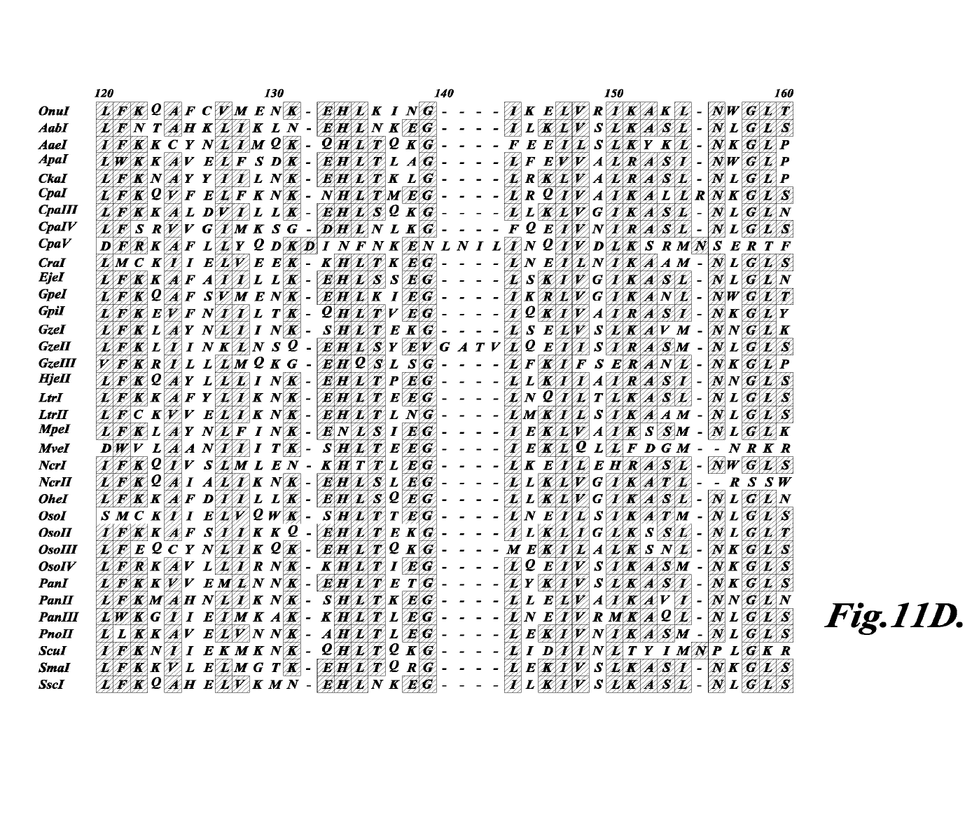

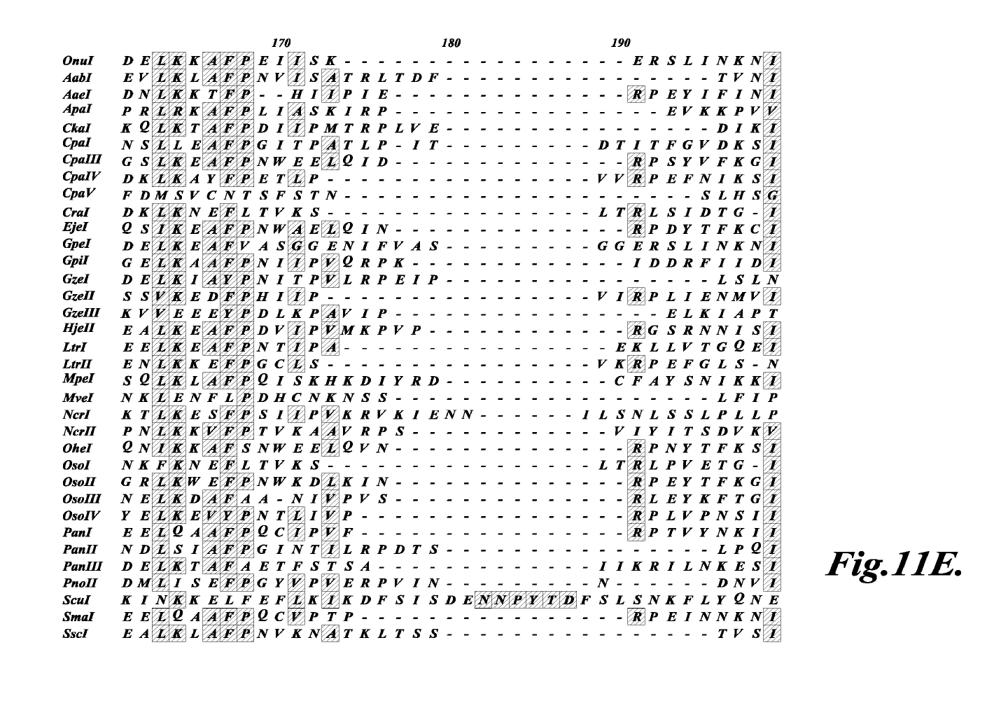

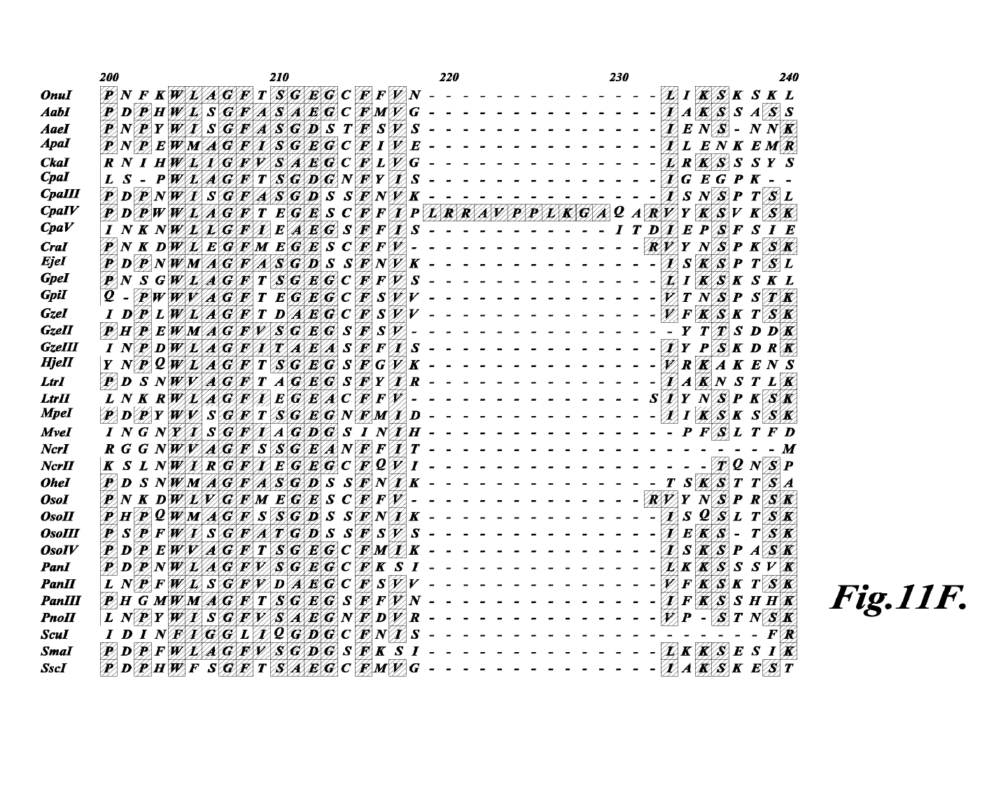

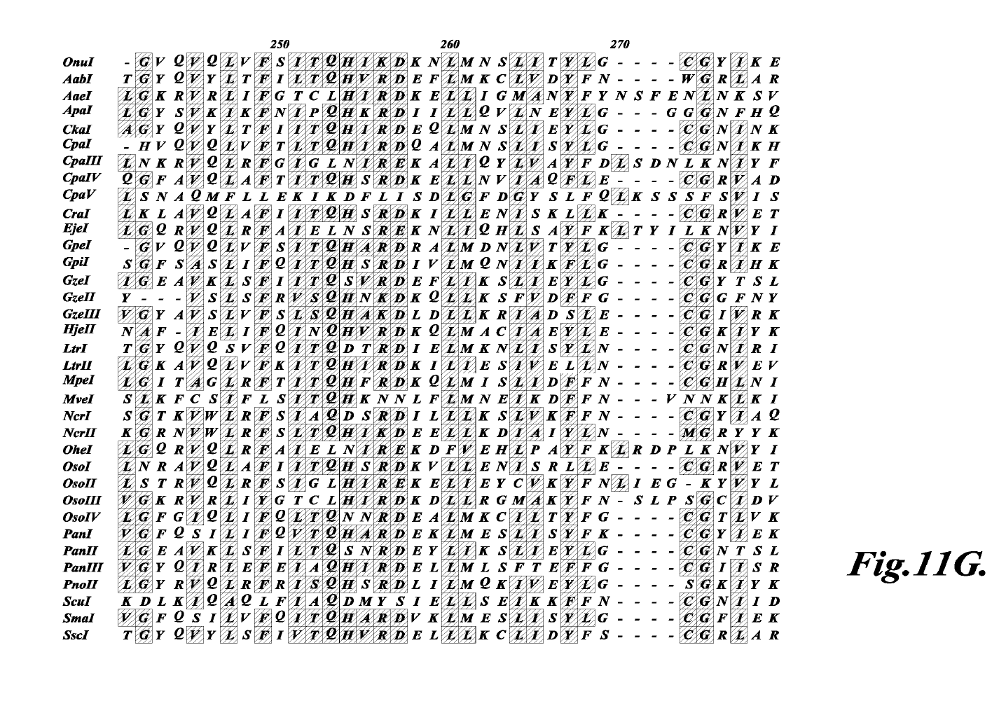

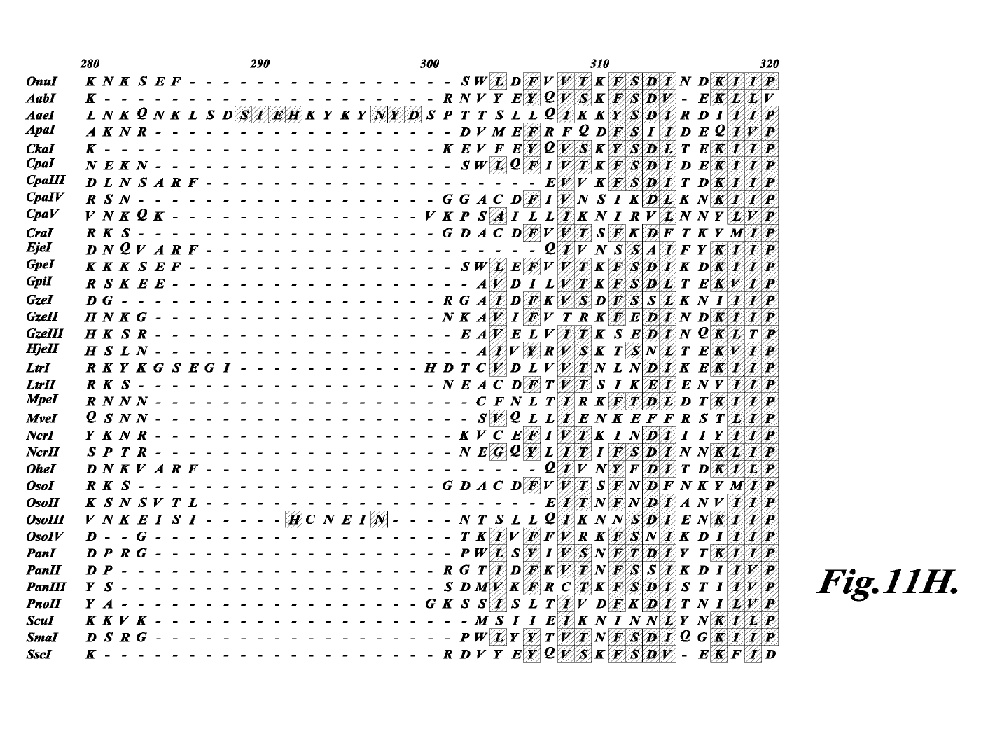

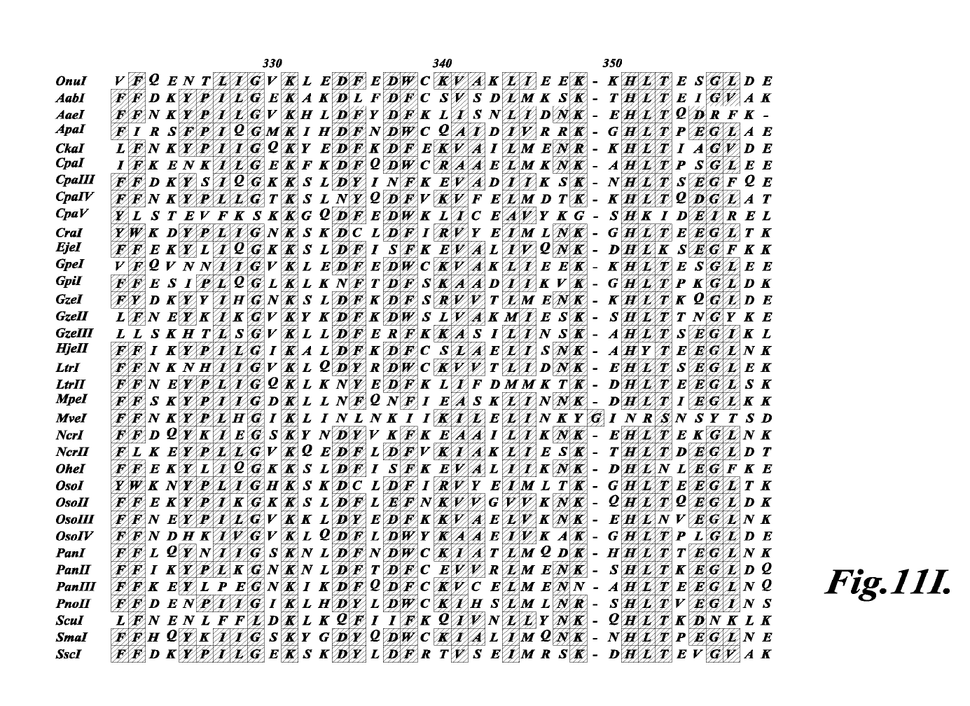

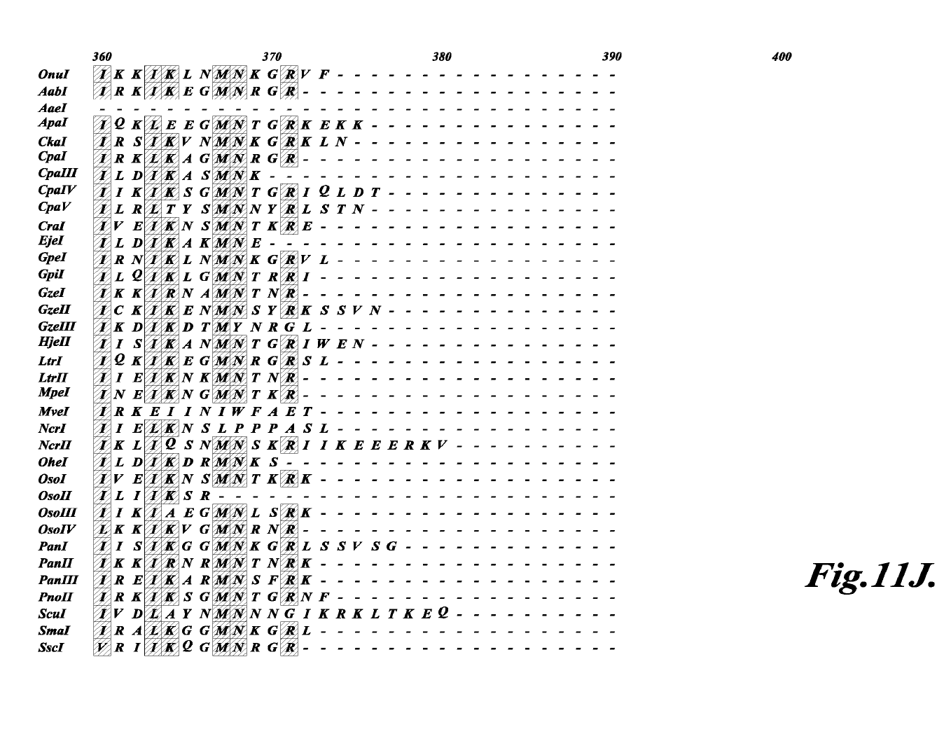

Homologues of I-OnuI can be identified by methods well known in the art. For example, sequence homology searches using I-OnuI as a query can be performed using the NCBI BLAST server (Altschul et al., J. Mol. Biol. 215:403-410, 1990), using the BLASTP protein-protein alignment algorithm, using the NCBI-curated “non-redundant” protein databases; this resource corresponds to all current GenBank, RefSeq Nucleotides, EMBL (European Molecular Biology Laboratories), DDBJ (Databank of Japan), PDB (Protein Databank) and metagenomic sequences. For these searches, the default parameters for the search algorithm (including scoring matrix, gap penalties and compositional adjustments) are systematically varied until all recognizable homologues (corresponding to at about 25% or greater sequence identity extending over at least 200 amino acids and including both ‘LAGLIDADG’ sequence motifs) are identified. The present disclosure has determined that in certain embodiments, homologues will display highly conserved (greater than 50% sequence identity) to the LAGLIDADG motifs (amino acid residues 12 to 24 of SEQ ID NO: 35 and amino acid residues 170 to 181 of the I-OnuI amino acid sequence in SEQ ID NO: 35. In addition, homologues of I-OnuI will demonstrate high amino acid conservation (greater than 50% sequence identity) within a “Loop” sequence adjacent to the first LAGLIDADG helix corresponding to amino acid residues 97 to 103 of SEQ ID NO: 35. Still further, homologous of I-OnuI will demonstrate an overall spacing between the end and beginning of the two LAGLIDADG motifs of between about 162 and 182 amino acid residues (See FIG. 11). Individual homologues can display lower sequence identity at one of the regions described above, but conservation of the key residues involved in catalysis in the two separate LAGLIDADG motifs (corresponding to E22 or D22 in SEQ ID NO: 35 (E63 or D63 in FIG. 11) and E178 or D178 in SEQ ID NO: 35 (E246 or D246 in FIG. 11)) in the best clustered alignment of I-OnuI homologues allows their identification.

Homologues of I-OnuI (SEQ ID NO: 35) identified to date using the above criteria include, for example and not limitation, I-AabI (SEQ ID NO: 52), I-AaeI (SEQ ID NO: 53), I-ApaI (SEQ ID NO: 54), I-CkaI (SEQ ID NO: 55), I-CpaI (SEQ ID NO: 56), I-CapIII (SEQ ID NO: 57), I-CapIV (SEQ ID NO: 58), I-CpaV (SEQ ID NO: 59), I-CraI (SEQ ID NO: 60), I-EjeI (SEQ ID NO: 61), I-GpeI (SEQ ID NO: 62), I-GpiI (SEQ ID NO: 63), I-GzeI (SEQ ID NO: 64), I-GzeII (SEQ ID NO: 65), I-GzeIII (SEQ ID NO: 66), I-HjeII (SEQ ID NO: 67), I-LtrI (SEQ ID NO: 68), I-LtrII (SEQ ID NO: 69), I-MpeI (SEQ ID NO: 70), I-MveI (SEQ ID NO: 71), I-NcrI (SEQ ID NO: 72), I-NcrII (SEQ ID NO: 73), I-OheI (SEQ ID NO: 74), I-OsoI (SEQ ID NO: 75), I-OsoII (SEQ ID NO: 76), I-OsoIII (SEQ ID NO: 77), I-OsiIV (SEQ ID NO: 78), I-PanI (SEQ ID NO: 79), I-PanII (SEQ ID NO: 80), I-PanIII (SEQ ID NO: 81), I-PnoI (SEQ ID NO: 82), I-ScuI (SEQ ID NO: 83), I-SmaI (SEQ ID NO: 85), or I-SscI (SEQ ID NO: 86) as shown in FIG. 11. Additional homologues of I-OnuI can be identified as above. Newly determined microbial and metagenomic sequence databases are expected to contain additional homologues of I-OnuI that can also be identified as above and can be used as embodiments in the methods described herein.

Using a computational model program or algorithm the crystal structure of I-OnuI bound to its DNA target site was used to determine information about the conformation of the I-OnuI homing nuclease nucleic acid binding site, including the detection, identification and optimization of contact points between individual I-OnuI domains (which can be used to create hybrid endonucleases that contain novel pairings of those domains) or between I-OnuI domains and substrate nucleic acid molecules (which can be used to create novel protein DNA contacts and associated altered DNA binding and cleavage specificities). Similar methods and information were obtained for I-LtrI and can be obtained for any I-OnuI homologue, using a homology model of that homologue. A “homologue model” is a three dimensional model of a protein/DNA complex that is based upon the crystallographic structure of I-OnuI bound to its DNA target site. A “contact point” refers to the point at which the protein domains of I-OnuI, or a homologue thereof, or the protein domains of I-OnuI, or a homologue thereof, and its nucleic acid substrate interact. Such contact points are formed as a result of specific binding between two protein domains of I-OnuI, or a homologue thereof, or between protein domains of I-OnuI, or a homologue thereof, and a nucleic acid substrate molecule. Other amino acids within the interface can also be modified to enhance or improve the interaction between protein domains of I-OnuI, or a homologue thereof, or between protein domains of I-OnuI, or a homologue thereof, and nucleic acid molecules. “Interface” refers to the amino acids between protein domains of I-OnuI, or a homologue thereof, or between protein domains of I-OnuI, or a homologue thereof, and a nucleic acid molecule that form contact points, as well as those amino acids that are adjacent to contact points and along the planar surface between protein domains of I-OnuI, or a homologue thereof, or between protein domains of I-OnuI, or a homologue thereof, and nucleic acid molecules.

More importantly, the algorithm or program will allow the identification of either potential contact points or residues that are not properly interacting with a nucleic acid target sequence or other residues between protein domains of I-OnuI, or a homologue thereof, or between protein domains of I-OnuI, or a homologue thereof, and nucleic acid molecules that are inhibiting or reducing the overall interaction. Thus, the program or algorithm can identify potential contact points between protein domains of I-OnuI, or a homologue thereof, or between protein domains I-OnuI, or a homologue thereof, and nucleic acid molecules and/or identifying amino acids along the interface that can be modified to improve the interface (that is improve the interaction between protein domains of I-OnuI, or a homologue thereof, or between protein domains of I-OnuI, or a homologue thereof, and a nucleic acid target sequence.

Points at which the unmodified I-OnuI, or a homologue thereof, usually directly contacts a specific nucleic acid sequence (which may be different than the target sequence), but which are not contact points with the target sequence, are characterized as “potential contact points.” A potential contact point can refer to one or more amino acids that usually directly interact with a nucleic acid sequence but are not stably interacting with the target sequence because a strong or stable enough chemical bond cannot be formed between the amino acid(s) and the sequence. As a result, there is no contact point because of improper or inadequate bonding. The inability to bind can be due to chemical constraints (incompatible reaction groups) or proximity issues (too far or too close together). A specific chemical constraint can involve amino acids that repel each other or attract each other because of chemical charges. A specific proximity issue is when there is steric hindrance between either amino acids or between an amino acid and the target sequence, which precludes or interferes with proper chemical bonding. Alternatively, an amino acid(s) can be too far from the target sequence to create an interface. In such cases, there is a gap between the two, which can be reduced or eliminated to create a contact point.

I-OnuI, a I-OnuI homologue, or a hybrid of two I-OnuI or I-OnuI homologue domains, can be modified through one or more amino acid changes, including rotameric changes, to create an actual contact point between the I-OnuI, or homologue thereof, protein's nucleic acid binding domain and the target sequence. The I-OnuI or homologue thereof can also be modified through one or more amino acid changes to improve the interface between the protein and the nucleic acid. Methods for making these amino acid changes are well known to the skilled artisan and are not consider a part of the present disclosure.

An amino acid change is a modification that is a substitution, deletion, or addition of 1, 2, 3, 4, 5, 6, 7, 8, 9, 10, 11, 12, 13, 14, 15, 16, 17, 18, 19, 20 or more contiguous or non-contiguous amino acids. Therefore, the present disclosure provides for the identification of an amino acid change that creates or enhances a contact point or the interface between individual protein domains of I-OnuI, or a homologue thereof, or between protein domains of I-OnuI, or a homologue thereof, and nucleic acid molecules, which can further provide a design for a modified I-OnuI polypeptide, or a homologue thereof. Enhancing a contact point or the interface means that a point between individual protein domains or between protein domains and nucleic acid molecules is made more chemically favorable, which includes reducing entropy, increasing stability, and reducing any steric hindrance.

Amino acid changes to create 1, 2, 3, 4, 5, 6, 7, 8, 9, 10, 11, 12, 13, 14, 15, 16, 17, 18, 19, 20, 21, 22, 23, 24, 25 or more interfaces may be determined empirically or computationally, or both. A change that is determined computationally refers to the use of a computer program or algorithm to identify amino acid changes that would create a desired contact point or improve or enhance the interface. Such programs are well known in the art. In some embodiments, the change is identified based on other polypeptides that interact with a site of similar sequence. Parameters known to those of ordinary skill in the art can be employed to guide the program or algorithm, such as sequence alignments, three-dimensional structural alignments, calculations of molecular interaction energies, and docking scores based on molecular complementarity.

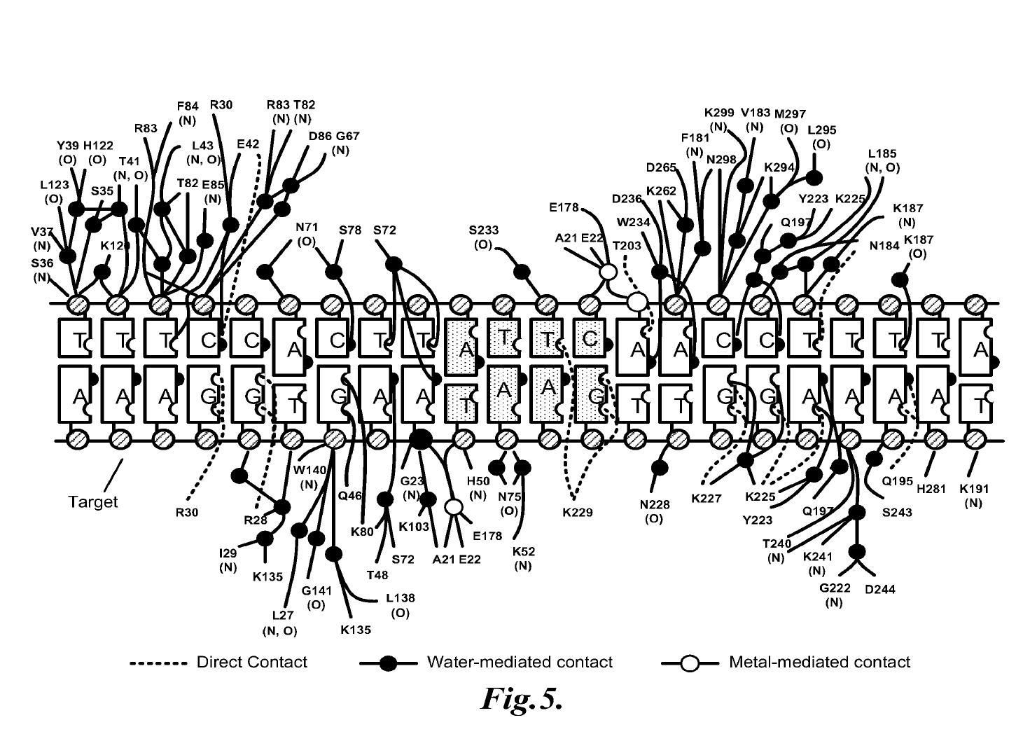

Amino acid side chains from the two structurally independent, anti-parallel β-sheets (one from each protein domain of I-OnuI, or a homologue thereof), can be used to contact nucleotide bases within the major groove, at positions flanking the central four base pairs (FIG. 5). The 22 base pair target site (SEQ ID NO: 1 and SEQ ID NO: 2), as shown in the illustration, was determined to be recognized by a combination of at least 22 mediated contacts between amino acid side chains and individual DNA bases, at least 14 additional water mediated contacts between amino acid side chains and individual DNA bases, and at least 30 contacts between protein and the DNA phosphoribose backbone (mostly water mediated). Further, at least 40 amino acid residues were found to be involved in direct or water-mediated contacts to the DNA target; these represent the first shell of amino acid residues that can be exploited for a redesign or selection to either improve the binding and enzyme activity of the I-OnuI endonuclease with its wild-type target sequence or to modify the binding site recognized and cleaved by the I-OnuI endonuclease. Similar methods can be used to either improve or alter the binding specificity and/or activity of a I-OnuI endonuclease homologue.

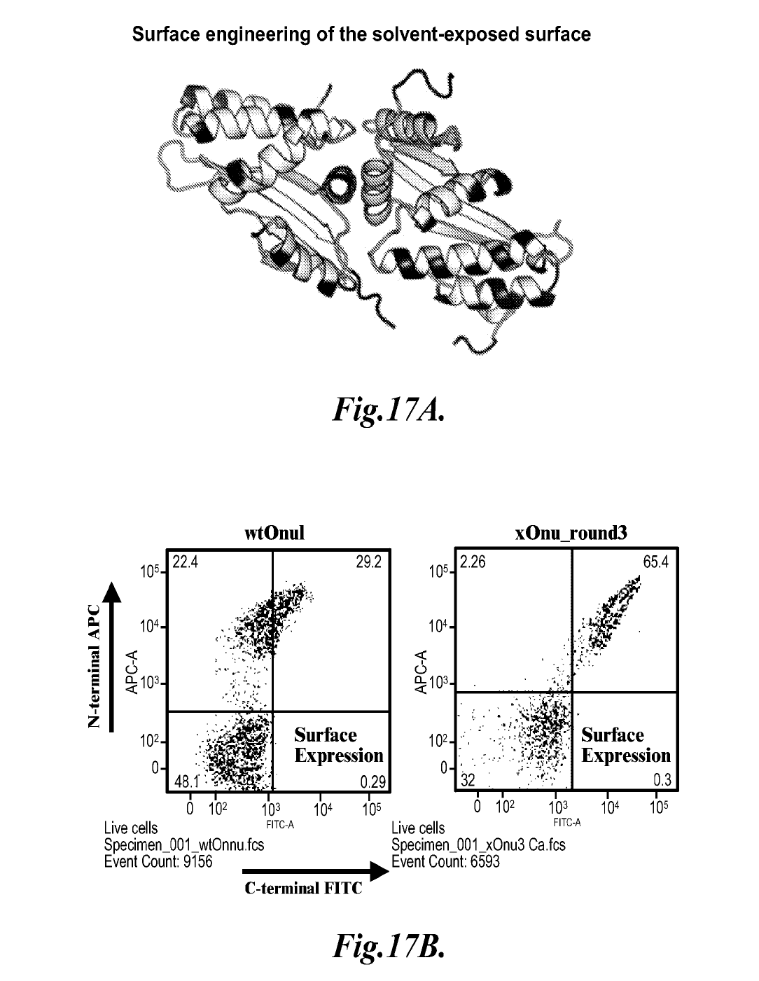

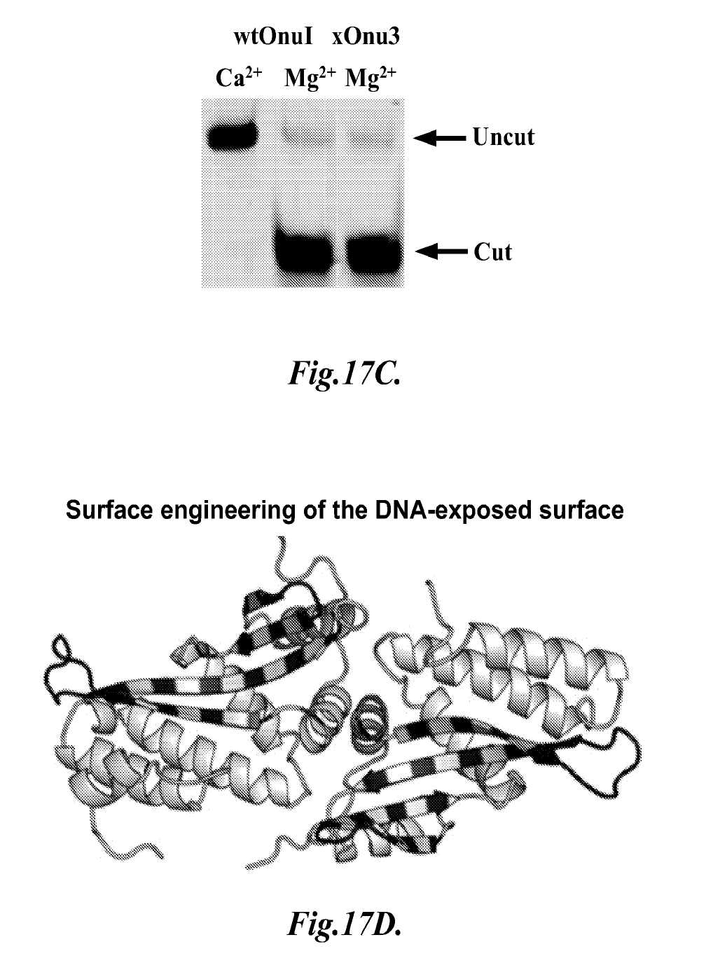

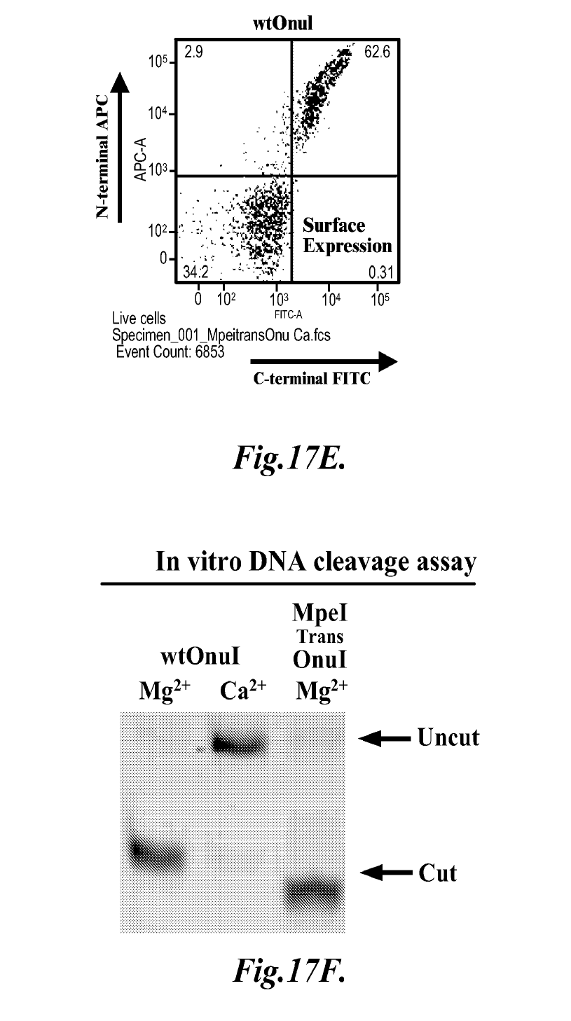

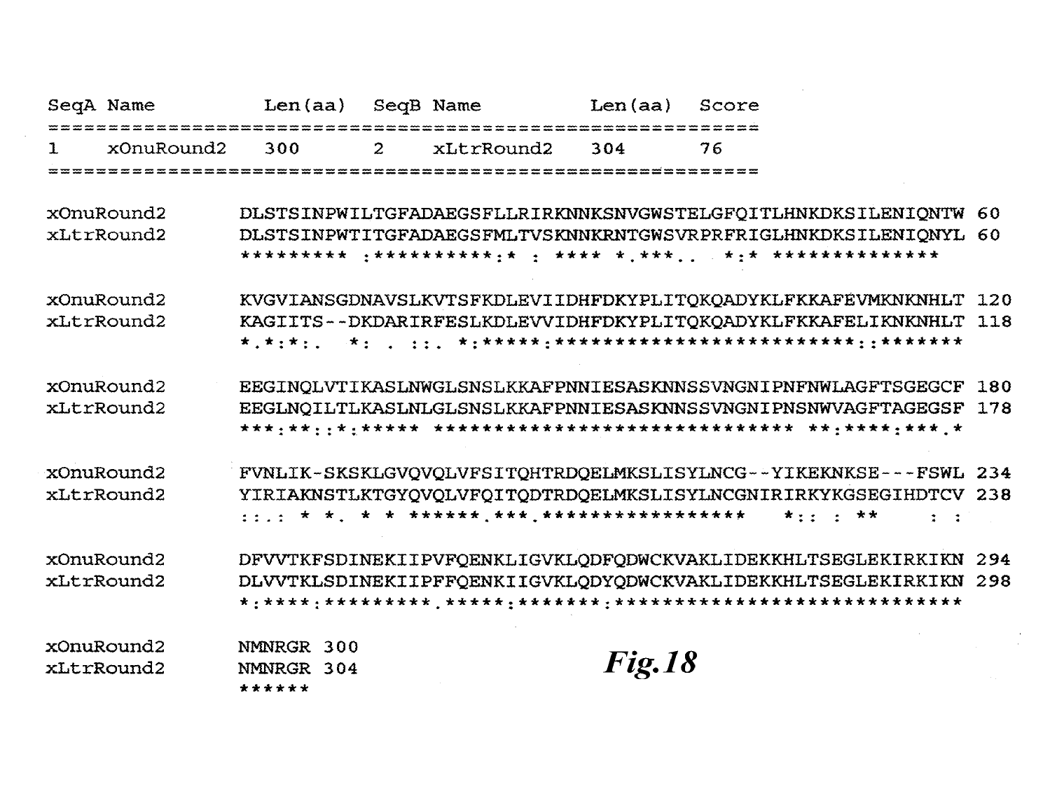

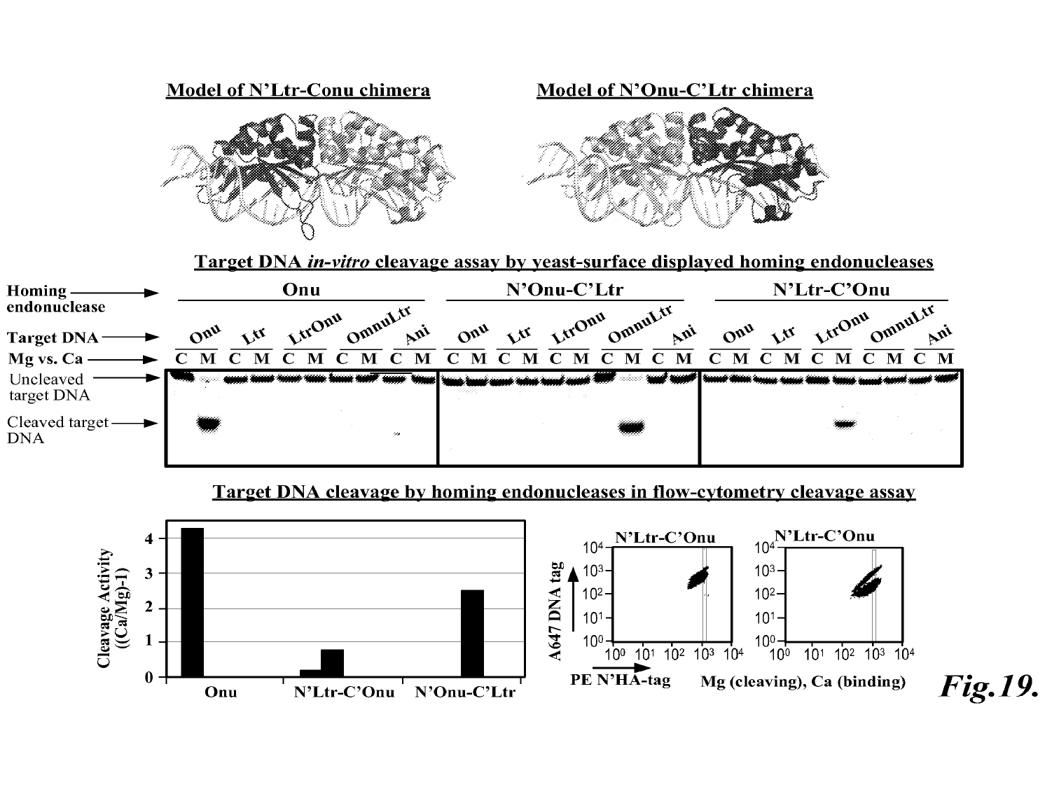

“Hybrid” homing endonuclease as used herein is a protein scaffold that contains distinct, recognizable elements of sequence or structure contributed by two or more separate wild-type (i.e., naturally occurring) homing endonucleases. A hybrid homing endonuclease can comprise any of the following combinations of protein elements contributed by separate wild-type homing endonuclease scaffolds: a) a fusion of two separate homing endonuclease domains (such as that previously described for “E-DreI” (now termed “H-DreI” for “hybrid I-DmoI/I-CreI”) (Chevalier et al., Mol. Cell. 10:895-905, 2002) and as illustrated in FIG. 19; b) a substitution of a peptide linker sequence from one homing endonuclease to connect the individual domains of a different homing endonuclease; c) a substitution of a DNA-contacting peptide or loop from one endonuclease into the comparable region of a different homing endonuclease; d) the substitution of surface-displayed residues from one homing endonuclease onto comparable positions of a different homing endonuclease as illustrated in FIG. 17; e) a fusion of multiple peptide regions from two or more homing endonuclease regions, leading to a single active endonuclease scaffold, using processes of DNA shuffling, recombination and PCR assembly from wild-type homing endonuclease coding sequences.

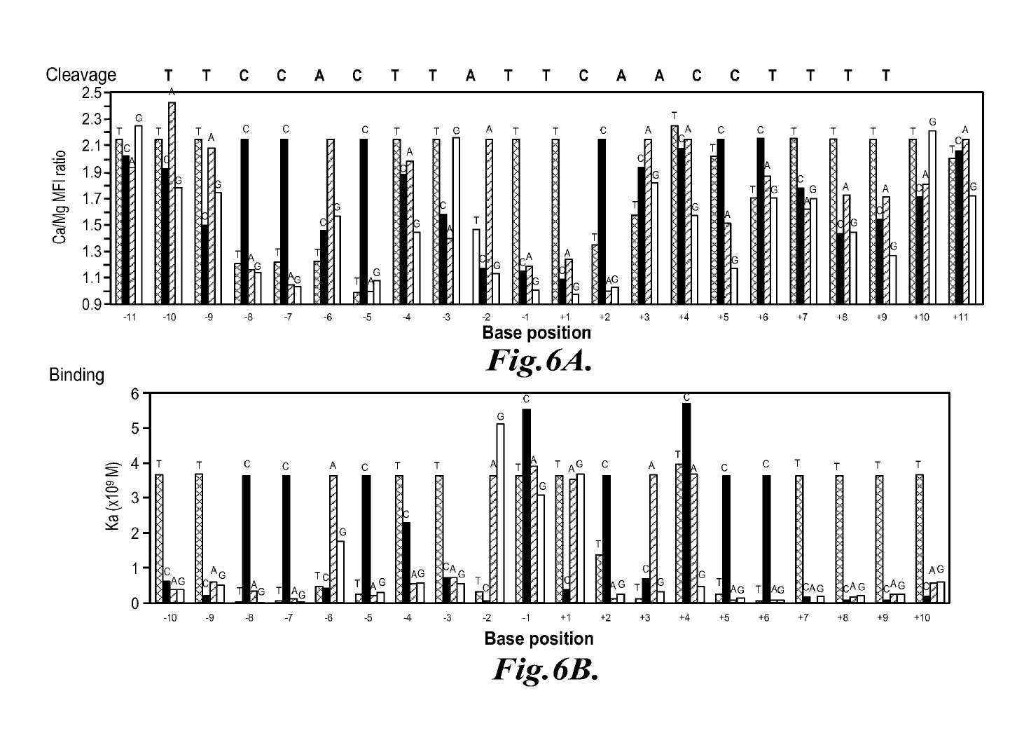

The binding and cleavage specificity of I-OnuI has been determined and is presented herein. The I-OnuI endonuclease was expressed using an expression vector expression/host cell system as described in detail in the examples. Methods for the expression and isolation of any endonuclease including I-OnuI, or a homologue thereof, are well known in the art. The binding site or target site for I-OnuI is set forth in FIGS. 5 and 6. The specificity profile of I-OnuI endonuclease was determined as described in Jarjour et al. (Nucl. Acids Res. 37:6871-6880, 2009, incorporated herein by reference in its entirety). The specificity profiles illustrated the ability of I-OnuI to bind and cleave a series of alternative DNA target sequences that each contain a single basepair mismatch at each of the 22 positions in the DNA recognition site. The analysis also indicated that the majority of specificity of DNA recognition was accomplished during DNA binding, rather than at the chemical step of DNA hydrolysis (although there were several individual basepair substitutions that did not affect binding, but inhibited subsequent cleavage). Those basepair substitutions that can be bound and cleaved as well (or at least with an efficiency greater than at least 50% that of the corresponding wild-type DNA base) include −6C, −4C, +4T and +4C. The overall specificity of the enzyme was therefore extremely high (at least 1 in 10¹⁰).

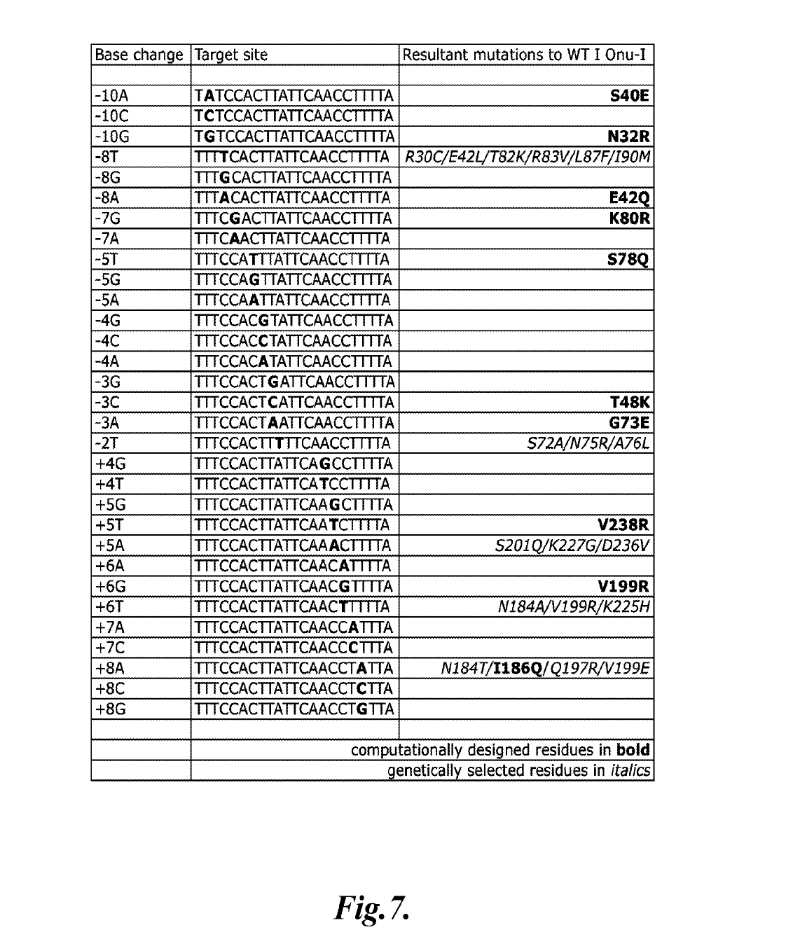

In one embodiment of the disclosure engineered variants of I-OnuI that can cleave altered DNA target sites (containing individual basepair substitutions) were identified using a combination of structure-based design and genetic selection for cleavage activity. The methods relied upon the identification of amino acid contact residues near each altered DNA basepair that was identified in the protein-DNA crystal structure and subsequent selection of surrounding amino acid substitutions (corresponding to a ‘pocket’ of protein side chains that surround the immediate contacting residue at each position in the protein-DNA interface). The analysis identified alternative amino acid identities that could form contacts to altered basepairs at each position in the DNA target, while modeling a conservatively flexible nucleotide and protein backbone. Methods, programs and algorithms capable of making these calculations and determinations are well known in the art. Subsequent creation of limited protein mutation libraries using known methods, and screening these libraries for DNA cleavage that leads to elimination of a bacterial reporter gene has produced active enzyme variants with desired specificities for many single base-pair substitutions in the wild-type target site of I-OnuI homing endonuclease. In particular, mutations of Serine (S) at position 40 to Glutamic acid (E) (S40E), Asparagine at position 32 to Arginine (R) (N32R), Glutamic acid (E) at position 42 to glutamine (Q) (E42Q), Lysine 80 at position 80 to Arginine (R) (L80R), Serine (S) at position 78 to Glutamine (Q) (S78Q), Threonine (T) at position 48 to Lysine (K) (T48K), Glycine (G) at position 73 to Glutamic acid (E) (G73E), Valine at position 238 to Arginine (R) (V238R), Valine at position 199 to Arginine (R) (V199R), and Isoleucine at position 186 to Glutamine (Q) (I186Q) were found to recognize variant DNA cleavage sites. (See FIG. 7 and SEQ ID NO: 35). In addition, in certain embodiments a combination of mutations were found to result in a variant I-OnuI endonuclease that recognized a variant DNA cleavage site. These combination include, for example, wherein Arginine (R) at position 30 of I-OnuI (SEQ ID NO: 35) was replace with Cysteine (C), Glutamic acid (E) at position 42 was replaced with Leucine (L), Threonine (T) at position 82 was replaced with Lysine (K), Arginine at position 83 was replace by Valine (V), Leucine (L) at position 87 was replaced by Phenylalanine (F), and Isoleucine (I) at position 90 was replaced with Methionine (M) (R30C/E42L/T82K/R83V/L87F/190M); wherein the Serine at position 72 was replaced by Alanine (A), Asparagine (N) at position 75 was replaced by Arginine (R), and Alanine (A) at position 76 was replaced by Leucine (L) (S72A/N75R/A76L); wherein the Serine (S) at position 201 was replaced by Glutamine (Q), Lysine (K) at position 227 was replace by Glycine (G) and Aspartic acid (D) at position 236 was replaced by Valine (S201Q/K227G/D236V); wherein the Asparagine (R) at position 184 was replaced by Alanine, Valine (V) at position 199 was replaced by Arginine (R), Lysine (K) at position 225 was replaced by Histidine (H) (N184A/V199R/K225H); and wherein the Asparagine (N) at position 184 was replaced by Threonine (T), Isoleucine at position 186 was replaced by Glutamine (Q), Glutamine (Q) at position 197 was replaced by Arginine (R), and Valine at position 199 was replaced by Glutamic acid (E) (N184T/I186Q/Q197R/V199E). The selection system was also modified to allow for the selection of an enzyme specificity as well as activity by selecting against enzyme variants that could still cleave the wild-type target site.

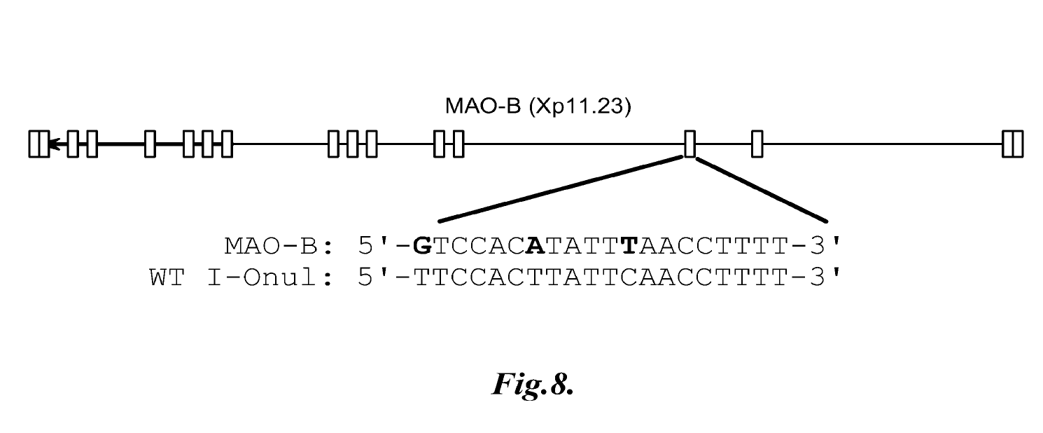

One of the main purposes of the present disclosure is to obtain a specific endonuclease that can cleave a specific site within a gene of interest. As such, a variant of I-OnuI was engineered to recognize and cleave a physiological target site in the human genome. The human monoamine oxidase B gene was selected because the gene includes a nucleotide sequence highly similar to the target nucleotide sequence of I-OnuI. See FIG. 8 and SEQ ID NO: 34. In addition, human monoamine oxidase B (MAO-B) catalyzes the deamination of a large number of biogenic amines in the brain and central nervous system, including serotonin, dopamine, and phenylethyamine (Shih et al., Annu. Rev. Neurosci. 22:197-217, 1999). Point mutations in MAO-B (as well as in monoamine oxidase A) are associated with many neurological and cognitive disorders, including Parkinson's Disease, compulsive and addictive behaviors, stress disorders, and aggressive behaviors (Shih et al., Annu. Rev. Neurosci. 22:197-217, 1999; Lew, Pharmacotherapy 27:155 S-160S, 2007). Although this gene is not typically a gene therapy target, MAO-B is under intense study (primarily using gene knockouts in mice) to elucidate the role of wild-type and mutant enzymes in neurological function and behavior (Grimsby et al., Nature Genet. 17:1-5, 1997). Using the crystal structure obtained for I-OnuI and its target DNA sequence and the models for the I-OnuI binding sites, variants of I-OnuI have been engineered that target a unique sequence in the MAO-B target site. This variant I-OnuI can be used to direct homologous recombination in that gene locus, leading to targeted point mutations of the endogenous MAO-B gene and the development of neural cell lines for in vivo studies.

The ability to engineer a variant I-OnuI endonuclease to a new target site demonstrated the concept that nucleotide target sequences having at least 40% identity to the target sequence of I-OnuI, or a homologue thereof, in a gene of interest can be selected and that a variant of I-OnuI or a homologue thereof (including hybrid endonucleases) can be made that can efficiently bind to and cleave the DNA within the gene at this specific site. Further, if the nucleotide sequence of a gene of interest is known the binding sites for each of the I-OnuI family of endonucleases can be searched to find a binding site sufficiently related (at least 40% identity) that can be modified by the methods discloses herein to target a variant binding site within the gene of interest. As such, the altered and hybrid endonucleases as described herein make possible targeted gene insertion or modification in a greater number of genes of interest. The family of I-OnuI endonuclease and its homologues provided a broad set of protein scaffolds for the design and selection of additional DNA cleavage specificities.

As such, the method for selecting an engineered I-OnuI endonuclease, a I-OnuI endonuclease homologue, or a hybrid of two I-OnuI or I-OnuI homologue domains with a target site modification from the wild-type and directed to a site within a gene of interest comprises the steps of; i) determining the target site nucleic acid sequence for the I-OnuI endonuclease, I-OnuI endonuclease homologues, and hybrids of two I-OnuI or I-OnuI homologue domains; ii) searching a nucleic acid database for a nucleotide sequence at least 40% identical to the target site nucleotide sequence of the I-OnuI endonuclease or I-OnuI homologue endonuclease, or a hybrid of two I-OnuI or I-OnuI homologue endonuclease domains; iii) selecting a gene of interest having the nucleotide sequence at least 40% identical to the target site nucleic acid sequence of the I-OnuI endonuclease, I-OnuI homologue endonuclease, or the hybrids of two I-OnuI or I-OnuI homologue endonuclease domains; iv) mutating the I-OnuI endonuclease, I-OnuI homologue endonuclease, or the hybrid of two I-OnuI or I-OnuI homologue endonuclease domains at amino acid residues that have been determined to be direct contact residues, backbone contact residues, or water-mediated contact residues with the nucleic acid sequence of the gene of interest having at least 40% identity to the target site of the I-OnuI endonuclease, I-OnuI homologue endonuclease, or the hybrid of two I-OnuI or I-OnuI homologue endonuclease domains to form a library of variant or engineered I-OnuI endonuclease, I-OnuI homologue endonuclease or variants thereof, or hybrids of two I-OnuI or I-OnuI homologue endonuclease domains; v) expressing the library of engineered I-OnuI endonuclease, I-OnuI homologue endonuclease, or hybrids of two I-OnuI or I-OnuI homologue endonuclease domains; vi) screening the library of variant or engineered I-OnuI endonuclease, I-OnuI homologue endonuclease and variants thereof, or hybrids of two I-OnuI or I-OnuI homologue endonuclease domains for binding activity to the nucleic acid sequence in the selected gene of interest and the cleavage activity for the target sequence in the selected gene of interest; and vii) selecting the variant or engineered I-OnuI endonuclease, I-OnuI endonuclease homologue or variant thereof, or the hybrid of two I-OnuI or I-OnuI homologue endonuclease domains with the highest binding and cleavage activity for the target sequence in the selected gene of interest.

In another embodiment, the method can be carried out starting from a selected gene of interest and searching a data base of I-OnuI endonuclease and I-OnuI endonuclease homologue target sequences. Once the target sequences of the I-OnuI endonuclease or the I-OnuI endonuclease homologue, or the hybrid of two I-OnuI or I-OnuI homologue domains with the best match with a nucleotide sequence within the target gene has been selected steps iv) through viii) above can be repeated and the variant or engineered I-OnuI endonuclease, I-OnuI endonuclease homologue or variant thereof, or hybrid of two I-OnuI or I-OnuI homologue domains selected for the highest binding activity and/or cleavage activity for the target sequence in the gene of interest. Once selected the variant or engineered I-OnuI endonuclease, I-OnuI homologue or variant thereof, and/or the engineered hybrid of two I-OnuI or I-OnuI homologue endonuclease domains can be used as set forth below. The term “engineered I-OnuI endonuclease, or homologues and hybrids thereof” or “variants of I-OnuI” as used herein include the engineered I-OnuI endonucleases, engineered I-OnuI homologues, and/or the engineered hybrid of two I-OnuI or I-OnuI homologue endonuclease domains set forth above.

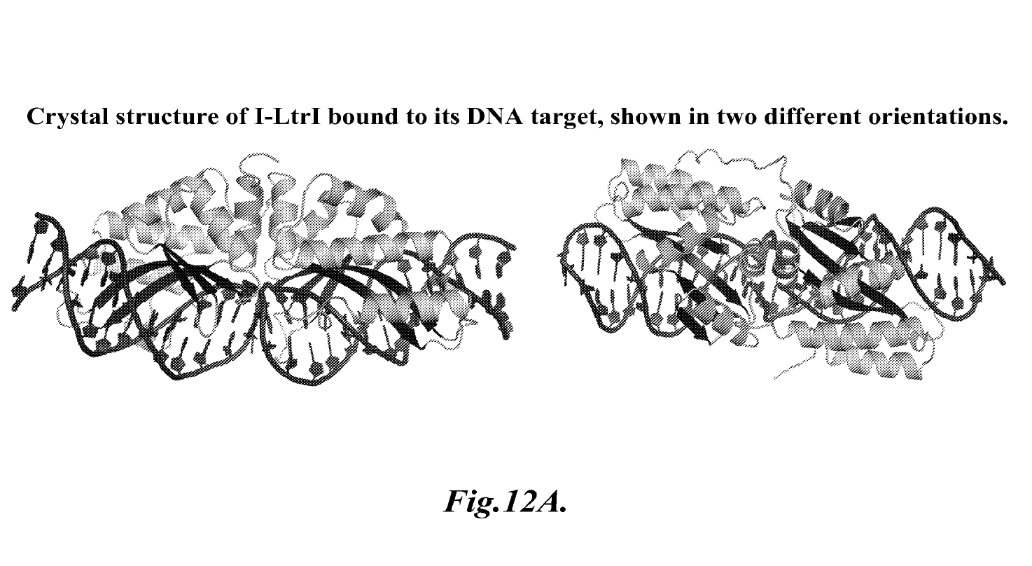

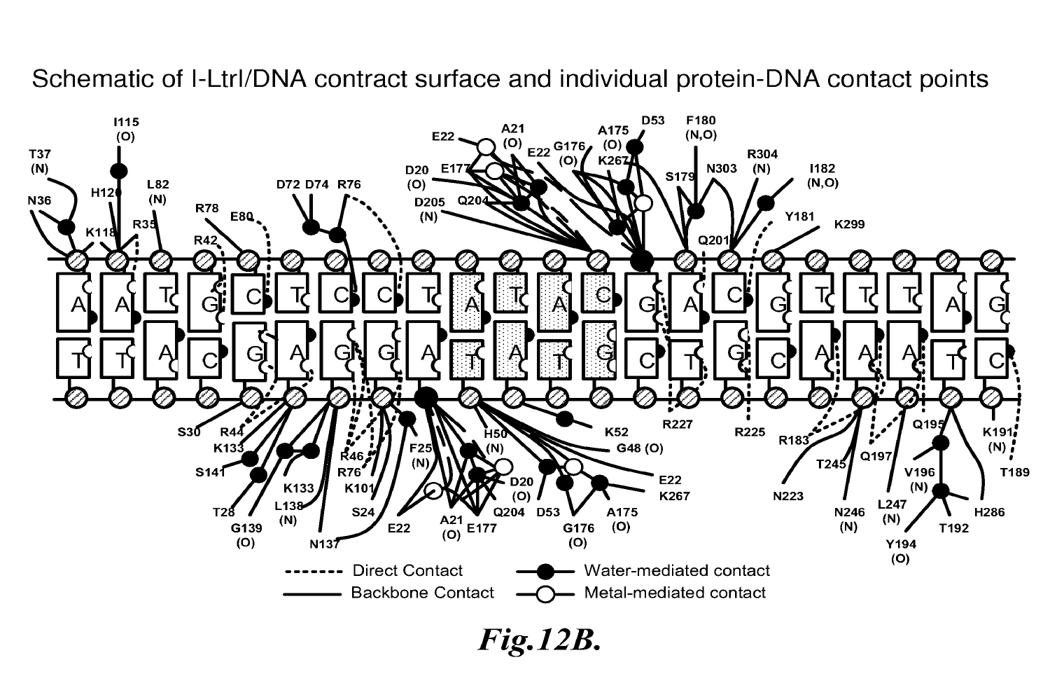

Variants of I-OnuI, including variants of I-OnuI and I-OnuI homologues, e.g., I-LtrI, and hybrids of two I-OnuI or I-OnuI homologue domains, that can cleave altered DNA target sites (containing individual basepair substitutions) were identified using a combination of structure-based design and genetic selection for cleavage activity. The method relied upon identification of amino acid contact residues near each altered DNA basepair that were identified in the protein-DNA crystal structure (such as, for example, illustrated in FIG. 5 and FIG. 12B) and subsequent selection of surrounding amino acid substitutions (corresponding to a ‘pocket’ of protein side chains that surround the immediate contacting residue at each position in the protein-DNA interface). The analysis can identify alternative amino acid identities that can form contacts to altered basepairs at each position in the DNA target, while modeling a conservatively flexible nucleotide and protein backbone. Subsequent creation of limited protein mutation libraries and screening these libraries for DNA cleavage that leads to elimination of a bacterial reporter gene has produced active enzyme variants with desired specificities for many single base-pair substitutions in the wild-type target site of I-OnuI homing endonuclease (See, for example, FIG. 7). The selection system was also modified to allow for selection of enzyme specificity as well as activity by selecting against enzyme variants that can still cleave the wild-type target site. In one embodiment the engineering of a variant of I-OnuI against a physiological target site in the human genome (the monoamine oxidase B (MAO-B) gene) is described below.

Provided herein are also vectors comprising the nucleic acid sequence that encodes the variant engineered I-OnuI endonuclease, I-OnuI endonuclease homologue, or engineered hybrid of two I-OnuI and/or I-OnuI homologue endonuclease domains of the present disclosure. A nucleic acid encoding one or more engineered I-OnuI, or engineered I-OnuI homologue or hybrid thereof, can be cloned into a vector for transformation into prokaryotic or eukaryotic cells for replication and/or expression. Vectors can be prokaryotic vectors, e.g., plasmids, or shuttle vectors, insect vectors, or eukaryotic vectors. A nucleic acid encoding an engineered I-OnuI, or homologue or hybrid thereof, as disclosed herein can also be cloned into an expression vector, for administration to a plant cell, animal cell, a mammalian cell or a human cell, fungal cell, bacterial cell, or protozoan cell.

To obtain expression of a cloned gene or nucleic acid, sequences encoding an engineered I-OnuI, or homologue or hybrid thereof, is typically subcloned into an expression vector that contains a promoter to direct transcription. Suitable bacterial and eukaryotic promoters are well known in the art and described, e.g., in Sambrook et al., Molecular Cloning, A Laboratory Manual (2nd ed. 1989; 3rd ed., 2001); Kriegler, Gene Transfer and Expression. A Laboratory Manual (1990); and Current Protocols in Molecular Biology (Ausubel et al., supra.) Bacterial expression systems for expressing the engineered I-OnuI, and homologue or hybrid thereof, are available in, e.g., E. coli, Bacillus sp., and Salmonella (Palva et al., Gene 22:229-235, 1983). Kits for such expression systems are commercially available. Eukaryotic expression systems for mammalian cells, yeast, and insect cells are well known by those of skill in the art and are also commercially available.

The promoter used to direct expression of an engineered I-OnuI, or homologue or hybrid thereof encoding nucleic acid depends on the particular application. For example, a strong constitutive promoter is typically used for expression and purification of a variant or engineered I-OnuI, or homologue or hybrid thereof. In contrast, when an engineered I-OnuI, or homologue or hybrid thereof, is administered in vivo for gene regulation, either a constitutive or an inducible promoter is used, depending on the particular use of the engineered I-OnuI, or homologue or hybrid thereof. In addition, a promoter for administration of an engineered I-OnuI, or homologue or hybrid thereof, can be a weak promoter, such as HSV TK or a promoter having similar activity. The promoter typically can also include elements that are responsive to transactivation, e.g., hypoxia response elements, Gal4 response elements, lac repressor response element, and small molecule control systems such as tet-regulated systems and the RU-486 system (see, eg., Gossen and Bujard, Proc. Nat'l. Acad. Sci. 89:5547, 1992; Oligino et al., Gene Ther. 5:491-496, 1998; Wang et al., Gene Ther. 4:432-441, 1997; Neering et al., Blood 88:1147-1155, 1996; and Rendahl et al., Nat. Biotechnol. 16:757-761, 1998). The MNDU3 promoter can also be used, and is preferentially active in CD34⁺ hematopoietic stem cells.

In addition to the promoter, the expression vector typically contains a transcription unit or expression cassette that contains all the additional elements required for the expression of the nucleic acid in a host cell, either prokaryotic or eukaryotic. A typical expression cassette thus contains a promoter operably linked, e.g., to a nucleic acid sequence encoding the engineered endonuclease, and signals required, e.g., for efficient polyadenylation of the transcript, transcriptional termination, ribosome binding sites, or translation termination. Additional elements of the cassette may include, e.g., enhancers, and heterologous splicing signals.

The particular expression vector used to transport the genetic information into the cell is selected with regard to the intended use of the engineered I-OnuI, or homologue or hybrid thereof, e.g., expression in plants, animals, bacteria, fungus, protozoa, and the like. Standard bacterial expression vectors include plasmids such as pBR322-based plasmids, pSKF, pET23D, pBluescript® based plasmids, and commercially available fusion expression systems such as GST and LacZ. An exemplary fusion protein is the maltose binding protein, “MBP.” Such fusion proteins are used for purification of the engineered I-OnuI, or homologue or hybrid thereof. Epitope tags can also be added to recombinant proteins to provide convenient methods of isolation, for monitoring expression, and for monitoring cellular and subcellular localization, e.g., a nuclear localization signal (NLS), an HA-tag, c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMT010/A+, pMAMneo-5, bacculovirus pDSVE, pCS, pEF, and any other vector allowing expression of proteins under the direction of the SV40 early promoter, SV40 late promoter, metallothionein promoter, murine mammary tumor virus promoter, Rous sarcoma virus promoter, polyhedrin promoter, elongation factor 1 promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with an engineered I-OnuI, or homologues thereof, encoding sequence under the direction of the polyhedrin promoter or other strong baculovirus promoters.

The elements that are typically included in an expression vector also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (Guide to Protein Purification, in Methods in Enzymology, vol. 182 (Deutscher, ed., 1990)). Transformation of eukaryotic and prokaryotic cells are performed according to standard techniques well known to the skilled artisan.

Any of the well known procedures for introducing foreign nucleotide sequences into host cells can be used. These include the use of calcium phosphate transfection, polybrene, protoplast fusion, electroporation, ultrasonic methods (e.g., sonoporation), liposomes, microinjection, naked DNA, plasmid vectors, viral vectors, both episomal and integrative, and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

Nucleic acids encoding an engineered I-OnuI, or homologue or hybrid thereof, as described herein and delivery to cells can use conventional viral and non-viral based gene transfer methods (e.g., mammalian cells) and target tissues. Such methods can also be used to administer nucleic acids encoding an engineered I-OnuI, or homologue or hybrid thereof, to a cell in vitro. In certain embodiments, nucleic acids encoding an engineered I-OnuI, or homologue or hybrid thereof, are administered for in vivo or ex vivo gene modification uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids encoding an engineered I-OnuI, and homologue or hybrid thereof, include electroporation, lipofection, microinjection, biolistics, virosomes, liposomes, immunoliposomes, polycation or lipid: nucleic acid conjugates, naked DNA, artificial virions, and agent-enhanced uptake of DNA. Sonoporation using, e.g., the Sonitron 2000 system (Rich-Mar) can also be used for delivery of nucleic acids.

Lipofection is described in e.g., U.S. Pat. No. 5,049,386, U.S. Pat. No. 4,946,787; and U.S. Pat. No. 4,897,355) and lipofection reagents are sold commercially (e.g., Transfectam and Lipofectin™). Cationic and neutral lipids that are suitable for efficient receptor recognition of polynucleotides include those of Feigne, WO1991/17424, WO1991/16024. Delivery can be to cells (ex vivo administration) or target tissues (in vivo administration). The preparation of lipid: nucleic acid complexes, including targeted liposomes such as immunolipid complexes, is well known to one of skill in the art.

The use of RNA or DNA viral based systems for the delivery of nucleic acids encoding an engineered I-OnuI, or homologue or hybrid thereof, takes advantage of highly evolved processes for targeting a virus to specific cells in the body and trafficking the viral payload to the nucleus. Viral vectors can be administered directly to patients or they can be used to treat cells in vitro and the modified cells are administered to patients. Conventional viral based systems for the delivery of an engineered I-OnuI, or homologue or hybrid thereof, include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof.

In applications in which transient expression of an engineered I-OnuI, or homologue or hybrid thereof, is preferred, an adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene modification procedures. Construction of recombinant AAV vectors are described in a number of publications, including U.S. Pat. No. 5,173,414; Tratschin et al., Mol. Cell. Biol. 5:3251-3260, 1985; Tratschin et al., Mol. Cell. Biol. 4:2072-2081, 1984; Hermonat and Muzyczka, Proc. Natl. Acad. Sci. 81:6466-6470, 1984; and Samulski et al., J. Virol. 63:03822-3828, 1989. Recombinant adeno-associated virus vectors (rAAV) provide an alternative gene delivery systems based on the defective and nonpathogenic parvovirus adeno-associated type 2 virus.

Replication-deficient recombinant adenoviral vectors (Ad) can be produced at high titer and readily infect a number of different cell types. Most adenovirus vectors are engineered such that a transgene replaces the AdE1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity.

Packaging cells are used to form virus particles that are capable of infecting a host cell. Such cells include 293 cells, which package adenovirus, and 2 cells or PA317 cells, which package retrovirus. Viral vectors used in gene modification are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are typically supplied in trans by the packaging cell line. For example, AAV vectors used in gene modification typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line can also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

Gene modification vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene modification (e.g., via re-infusion of the transfected cells into the host organism) is well known to those of skill in the art. In a typical embodiment, cells are isolated from the subject organism, transfected with an engineered I-OnuI, or homologue or hybrid thereof, nucleic acid (gene or cDNA), and re-infused back into the subject organism (e.g., a patient). Various cell types suitable for ex vivo transfection are well known to those of skill in the art (see, e.g., Freshney et al., Culture of Animal Cells, A Manual of Basic Technique (5th ed. 2005)) and the references cited therein for a discussion of how to isolate and culture cells from a patient).

Vectors (e.g., retroviruses, adenoviruses, liposomes, and the like) containing an engineered I-OnuI, or homologue or hybrid thereof, nucleic acid can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions available (see, e.g., Remington The Science and Practice of Pharmacy, 21st ed., 2005).

DNA constructs may be introduced into the genome of a desired plant host by a variety of conventional techniques. For reviews of such techniques see, for example, Weissbach and Weissbach, Methods for Plant Molecular Biology (1988, Academic Press, N.Y.) Section VIII, pp. 421-463; and Grierson and Corey, Plant Molecular Biology (1988, 2d Ed.), Blackie, London, Ch. 7-9. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment.

Alternatively, the DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature and will not be described here.

Alternative gene transfer and transformation methods include, but are not limited to, protoplast transformation through calcium-, polyethylene glycol (PEG)- or electroporation-mediated uptake of naked DNA and electroporation of plant tissues. Additional methods for plant cell transformation include microinjection, silicon carbide mediated DNA uptake, and microprojectile bombardment.

The disclosed methods and compositions can be used to make genomic changes and/or to insert exogenous sequences into a predetermined location in a plant cell genome. This is useful inasmuch as expression of an introduced transgene into a plant genome depends critically on its integration site. Accordingly, genes encoding, e.g., nutrients, antibiotics or therapeutic molecules can be inserted, by targeted recombination, into regions of a plant genome favorable to their expression.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is a well known technique to the skilled artisan. Regeneration can also be obtained from plant callus, explants, organs, pollens, embryos or parts thereof.

Nucleic acids introduced into a plant cell can be used to confer desired traits on essentially any plant. A wide variety of plants and plant cell systems can be engineered for the desired physiological and agronomic characteristics described herein using the nucleic acid constructs of the present disclosure and the various transformation methods mentioned above. Typically, target plants and plant cells for modification include, but are not limited to, those monocotyledonous and dicotyledonous plants, such as crops including grain crops (e.g., wheat, maize, rice, millet, barley, and the like), fruit crops (e.g., tomato, apple, pear, strawberry, orange, and the like), forage crops (e.g., alfalfa, and the like), root vegetable crops (e.g., carrot, potato, sugar beets, yam, and the like), leafy vegetable crops (e.g., lettuce, spinach, and the like); flowering plants (e.g., petunia, rose, chrysanthemum, and the like), conifers and pine trees (e.g., pine fir, spruce, and the like); plants used in phytoremediation (e.g., heavy metal accumulating plants, and the like); oil crops (e.g., sunflower, rape seed (canola), camelina, and the like) and plants used for experimental purposes (e.g., Arabidopsis, and the like).

One of skill in the art will recognize that after the expression cassette is stably incorporated in a transgenic plant and confirmed to be operable, it can be introduced into other plants by sexual crossing. Any of a number of standard breeding techniques can be used, depending upon the species to be crossed.

A transformed plant cell, callus, tissue or plant may be identified and isolated by selecting or screening the engineered plant material for traits encoded by the marker genes present on the transforming DNA. For instance, selection may be performed by growing the engineered plant material on media containing an inhibitory amount of the antibiotic or herbicide to which the transforming gene construct confers resistance. Further, transformed plants and plant cells may also be identified by screening for the activities of any visible marker genes (e.g., the β-glucuronidase, green fluorescent protein, luciferase, B or Cl genes) that may be present on the recombinant nucleic acid constructs. Such selection and screening methodologies are well known to those skilled in the art.

Physical and biochemical methods also may be used to identify plant or plant cell transformants containing inserted gene constructs. These methods include but are not limited to: 1) Southern analysis or PCR amplification for detecting and determining the structure of the recombinant DNA insert; 2) Northern blot, siRNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct; 4) protein gel electrophoresis, Western blot techniques, immunoprecipitation, or enzyme-linked immunoassays, where the gene construct products are proteins. Additional techniques, such as in situ hybridization, enzyme staining, and immunostaining, also can be used to detect the presence or expression of the recombinant construct in specific plant organs and tissues. The methods for doing all these assays are well known to those skilled in the art.

Effects of gene manipulation using an engineered I-OnuI endonuclease, or homologue or hybrid thereof, disclosed herein can be observed by, for example, northern blots of the RNA (e.g., mRNA) isolated from the tissues of interest. Typically, if the amount of mRNA has increased, it can be assumed that the corresponding endogenous gene is being expressed at a greater rate than before. Other methods of measuring gene activity can be used.

Different types of enzymatic assays can be used, depending on the substrate used and the method of detecting the increase or decrease of a reaction product or by-product. In addition, the levels of and/or CYP74B protein expressed can be measured immunochemically, i.e., ELISA, RIA, EIA and other antibody based assays well known to those of skill in the art, such as by electrophoretic detection assays (either with staining or Western blotting). The transgene can be selectively expressed in some tissues of the plant or at some developmental stages, or the transgene may be expressed in substantially all plant tissues, substantially along its entire life cycle. However, any combinatorial expression mode is also applicable.

The present disclosure also encompasses seeds of the transgenic plants described above wherein the seed has the transgene or gene construct. The present disclosure further encompasses the progeny, clones, cell lines or cells of the transgenic plants described above wherein said progeny, clone, cell line or cell has the transgene or gene construct.

An important factor in the administration of polypeptide compounds, such as an engineered I-OnuI endonuclease, or homologue thereof, and a vector encoding an engineered I-OnuI, or homologue or hybrid thereof, is ensuring that the polypeptide or vector construct has the ability to traverse the plasma membrane of a cell, or the membrane of an intra-cellular compartment such as the nucleus. Proteins and other compounds such as liposomes have been described and are known to the skilled artisan, which have the ability to translocate polypeptides such as an engineered I-OnuI endonuclease, or homologue or hybrid thereof, across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. Toxin molecules also have the ability to transport polypeptides across cell membranes. Often, such molecules (called “binary toxins”) are composed of at least two parts: a translocation/binding domain or polypeptide and a separate toxin domain or polypeptide. Typically, the translocation domain or polypeptide binds to a cellular receptor, and then the toxin is transported into the cell. Typically, the translocation sequence is provided as part of a fusion protein. Optionally, a linker can be used to link the engineered I-OnuI endonuclease, or a homologue or hybrid thereof, and the translocation sequence. Any suitable linker can be used, e.g., a peptide linker.

The variant or engineered I-OnuI endonuclease, or homologue or hybrid thereof, and constructs encoding the variant or engineered I-OnuI endonuclease or homologue or hybrid thereof can also be introduced into an animal cell, preferably a mammalian cell, via a liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell, i.e., a variant or engineered I-OnuI endonuclease or homologue thereof or vector encoding the I-OnuI endonuclease or homologue or hybrid thereof. The liposome fuses with the plasma membrane, thereby releasing the variant or engineered I-OnuI endonuclease or homologue or hybrid thereof into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (in this case, the engineered I-OnuI endonuclease, or homologue or hybrid thereof) at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle. Liposome membranes can be constructed so that they become destabilized when the environment becomes acidic near the liposome membrane. When liposomes are endocytosed by a target cell, for example, they become destabilized and release their contents.

Such liposomes typically comprise a variant or engineered I-OnuI endonuclease, or homologue or hybrid thereof, and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes, and are well known in the art. Suitable methods include, for example, sonication, extrusion, high pressure/homogenization, microfluidization, detergent dialysis, calcium-induced fusion of small liposome vesicles and ether-fusion methods, all of which are known to those of skill in the art.

In certain embodiments, it is desirable to target liposomes using targeting moieties that are specific to a particular cell type, tissue, and the like. Targeting of liposomes using a variety of targeting moieties (e.g., ligands, receptors, and monoclonal antibodies) has been and methods for their construction and administration are well known to the skilled artisan. Standard methods for coupling targeting agents to liposomes can be used. These methods generally involve incorporation into liposomes of lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A.

The dose of a variant or engineered I-OnuI endonuclease, or a homologue or hybrid thereof, administered to a patient, or to a cell which will be introduced into a patient, in the context of the present disclosure, should be sufficient to effect a beneficial therapeutic response in the patient over time. In addition, particular dosage regimens can be useful for determining phenotypic changes in an experimental setting, e.g., in functional genomics studies, and in cell or animal models. The dose will be determined by the efficacy and K_{d} of the particular variant or engineered I-OnuI endonuclease, or homologue or hybrid thereof, employed, the nuclear volume of the target cell, and the condition of the patient, as well as the body weight or surface area of the patient to be treated. The size of the dose also will be determined by the existence, nature, and extent of any adverse side-effects that accompany the administration of a particular compound or vector in a particular patient.

The maximum therapeutically effective dosage of a variant or engineered I-OnuI endonuclease, or homologue or hybrid thereof, for approximately 99% binding to target sites is calculated to be in the range of less than about 1.5×10⁵ to 1.5×10⁶ copies of the specific variant or engineered I-OnuI endonuclease, or homologue or hybrid thereof molecule per cell. The appropriate dose of an expression vector encoding a variant or engineered I-OnuI endonuclease, or homologue or hybrid thereof, can also be calculated by taking into account the average rate of expression or the variant or engineered I-OnuI endonuclease, or homologue or hybrid thereof, from the promoter and the average rate of degradation of the variant or engineered I-OnuI endonuclease, or homologue or hybrid thereof, in the cell. In certain embodiments, a weak promoter such as a wild-type or mutant HSV TK promoter is used, as described above. The dose of variant or engineered I-OnuI endonuclease, or homologue or hybrid thereof, in micrograms is calculated by taking into account the molecular weight of the particular engineered I-OnuI endonuclease, or homologue or hybrid thereof, being employed.

In determining the effective amount of the I-OnuI endonuclease, variant or engineered I-OnuI endonuclease, or homologue or hybrid thereof, to be administered in the treatment or prophylaxis of disease, the physician evaluates circulating plasma levels of the I-OnuI endonuclease, or homologue or hybrid thereof, or nucleic acid encoding the variant or engineered I-OnuI endonuclease, or homologue or hybrid thereof, potential variant or engineered I-OnuI endonuclease, or homologue or hybrid thereof, toxicities, progression of the disease, and the production of anti-I-OnuI endonuclease, or homologue or hybrid thereof, antibodies. Administration can be accomplished via single or divided doses.

Pharmaceutical compositions and administration of a variant or engineered I-OnuI endonuclease, or homologue hybrid thereof, and expression vectors encoding a variant or engineered I-OnuI endonuclease, or homologue or hybrid thereof, can be administered directly to the patient for targeted single strand cleavage and/or recombination, and for therapeutic or prophylactic applications, for example, cancer, ischemia, diabetic retinopathy, macular degeneration, rheumatoid arthritis, psoriasis, HIV infection, sickle cell anemia, Alzheimer's disease, muscular dystrophy, neurodegenerative diseases, vascular disease, cystic fibrosis, stroke, and the like. Examples of microorganisms that can be inhibited by I-OnuI endonuclease, or variant or homologue or hybrid thereof, gene modification include pathogenic bacteria, e.g., Chlamydia, rickettsial bacteria, mycobacteria, staphylococci, streptococci, pneumococci, meningococci and gonococci, Klebsiella, Proteus, Serratia, Pseudomonas, Legionella, Diphtheria, Salmonella, bacilli, Cholera, tetanus, botulism, anthrax, plague, leptospirosis, Lyme disease bacteria and the like; infectious fungus, e.g., Aspergillus, Candida species and the like; protozoa such as sporozoa (e.g., Plasmodia), rhizopods (e.g., Entamoeba) flagellates (Trypanosoma, Leishmania, Trichomonas, Giardia, and the like), and the like; viral diseases, e.g., hepatitis (A, B, or C), herpes virus (e.g., VZV, HSV-1, HSV-6, HSV-11, CMV, and EBV), HIV, Ebola, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, poliovirus, rabies virus, and arboviral encephalitis virus, and the like.

Administration of therapeutically effective amounts is by any of the routes normally used for introducing a variant or engineered I-OnuI endonuclease, or homologue or hybrid thereof, or an expression vector encoding a variant or engineered I-OnuI endonuclease, or homologue or hybrid thereof, of the invention into ultimate contact with the tissue or cell type to be treated. The variant or engineered I-OnuI endonuclease, or homologue or hybrid thereof, is administered in any suitable manner, preferably with a pharmaceutically acceptable carrier. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remington The Science and Practice of Pharmacy, 21st ed., 2005, Lippincott Williams & Wilkins).

The variant or engineered I-OnuI endonuclease, or homologue or hybrid thereof, alone or in combination with other suitable components, can be made into an aerosol formulation (i.e., “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally.

The formulations of compounds can be presented in unit-dose or multi-dose sealed containers, such as ampules and vials. Injection solutions and suspensions can be prepared from sterile powders, granules, and tablets of the kind previously described.

The disclosed methods and variant or engineered I-OnuI endonuclease, or homologue or hybrid thereof, compositions for targeted cleaving one strand of a polynucleotide sequence can be used to induce mutations in a genomic sequence, e.g., by cleaving the DNA in the region of its genomic target sequence and initiating enzymatic events and subsequent mechanisms in the cell that lead to gene conversion and repair shifted to conservative, templated recombination pathways. The same methods can also be used to replace a wild-type sequence with a mutant sequence, or to convert one allele to a different allele.

Targeted DNA cleavage of an infecting or integrated viral genome can be used to treat viral infections in a host. Additionally, targeted DNA cleavage of a gene encoding a receptor for a virus can be used to block expression of such receptors, thereby preventing viral infection and/or viral spread in a host organism. Targeted mutagenesis of a gene encoding a viral receptor can be used to render the receptor unable to bind to virus, thereby preventing new infection and blocking the spread of existing infections. Non-limiting examples of viruses or viral receptors that may be targeted include herpes simplex virus (HSV), such as HSV-1 and HSV-2, varicella zoster virus (VZV), Epstein-Ban virus (EBV) and cytomegalovirus (CMV), HHV6 and HHV7. The hepatitis family of viruses includes hepatitis A virus (HAV), hepatitis B virus (HBV), hepatitis C virus (HCV), the delta hepatitis virus (HDV), hepatitis E virus (HEV) and hepatitis G virus (HGV). Other viruses or their receptors may be targeted, including, but not limited to, Picornaviridae (e.g., polioviruses, and the like); Caliciviridae; Togaviridae (e.g., rubella virus, dengue virus, and the like); Flaviviridae; Coronaviridae; Reoviridae; Birnaviridae; Rhabodoviridae (e.g., rabies virus, and the like); Filoviridae; Paramyxoviridae (e.g., mumps virus, measles virus, respiratory syncytial virus, and the like); Orthomyxoviridae (e.g., influenza virus types A, B and C, and the like); Bunyaviridae; Arenaviridae; Retroviradae; lentiviruses (e.g., HTLV-I; HTLV-II; HIV-1, HIV-II); simian immunodeficiency virus (SIV), human papillomavirus (HPV), influenza virus and the tick-borne encephalitis viruses. See, e.g., Fundamental Virology, 2nd Edition (Knipe et al., eds. 2001), for a description of these and other viruses.

In similar fashion, the genome of an infecting bacterium can be mutagenized by targeted DNA cleavage followed by templated recombination, to block or ameliorate bacterial infections. The disclosed methods for targeted homologous recombination can be used to replace any genomic sequence with a homologous, non-identical sequence. For example, a mutant genomic sequence can be replaced by its wild-type counterpart, thereby providing a method for treatment of, e.g., a genetic disease, an inherited disorders, cancer, and an autoimmune disease. In like fashion, one allele of a gene can be modified using the methods of targeted recombination disclosed herein.

Exemplary genetic diseases include, but are not limited to, achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, Canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GMI), hemochromatosis, the hemoglobin C mutation in the 6 codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, and X-linked lymphoproliferative syndrome (XLP, OMIM No. 308240).

Additional exemplary diseases that can be treated by targeted single DNA strand cleavage and/or targeted templated homologous recombination of the invention include acquired immunodeficiencies, lysosomal storage diseases (e.g., Fabry disease), mucopolysaccahidosis (e.g., Hunter's disease), hemoglobinopathies and hemophilias. In certain cases, alteration of a genomic sequence in a pluripotent cell (e.g., a hematopoietic stem cell) is desired. Methods for mobilization, enrichment and culture of hematopoietic stem cells are known in the art. Treated stem cells can be returned to a patient for treatment of various diseases including, but not limited to, SCID and sickle-cell anemia.

In many of these cases, a region of interest comprises a mutation, and the donor polynucleotide comprises the corresponding wild-type sequence. Similarly, a wild-type genomic sequence can be replaced by a mutant sequence, if such is desirable. For example, overexpression of an oncogene can be reversed either by mutating the gene or its control sequences with sequences that support a lower, non-pathologic level of expression. Any pathology dependent upon a particular genomic sequence, in any fashion, can be corrected or alleviated using the methods and compositions disclosed herein.

Targeted DNA cleavage and targeted template recombination can also be used to alter non-coding sequences (e.g., regulatory sequences such as promoters, enhancers, initiators, terminators, splice sites) to alter the levels of expression of a gene product. Such methods can be used, for example, for therapeutic purposes, functional genomics and/or target validation studies.

The variant or engineered I-OnuI, and homologues and hybrids thereof, compositions and methods described herein also allow for novel approaches and systems to address immune reactions of a host to, for example, allogeneic grafts. In particular, a major problem faced when allogeneic stem cells (or any type of allogeneic cell) are grafted into a host recipient is the high risk of rejection by the host's immune system, primarily mediated through recognition of the Major Histocompatibility Complex (MHC) on the surface of the engrafted cells. The MHC comprises the HLA class I protein (s) that function as heterodimers that are comprised of 3 common subunits and a variable subunit. It has been demonstrated that tissue grafts derived from stem cells that are devoid of HLA escape the host's immune response. Using the variant or engineered I-OnuI, and homologues and hybrids thereof, compositions and methods described herein, genes encoding HLA proteins involved in graft rejection can be cleaved, mutagenized or altered by templated recombination, in either their coding or regulatory sequences, so that their expression is blocked or they express a non-functional product. For example, by inactivating the gene encoding the common β subunit gene (β2 microglobulin) using a variant or engineered I-OnuI, and homologues thereof, as described herein, HLA class I can be removed from the cells to rapidly and reliably generate HLA class I null stem cells from any donor, thereby reducing the need for closely matched donor/recipient MHC haplotypes during stem cell grafting.

Inactivation of a gene (e.g., the β2 microglobulin or other gene) can be achieved, for example, by a single cleavage event, by cleavage followed by templated recombination, by targeted recombination of a missense or nonsense codon into the coding region, or by targeted recombination of an irrelevant sequence (i.e., a “stuffer” sequence) into the gene or its regulatory region, so as to disrupt the gene or regulatory region.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### EXAMPLES

### The Crystal Structure and Biochemical Characterization of I-OnuI

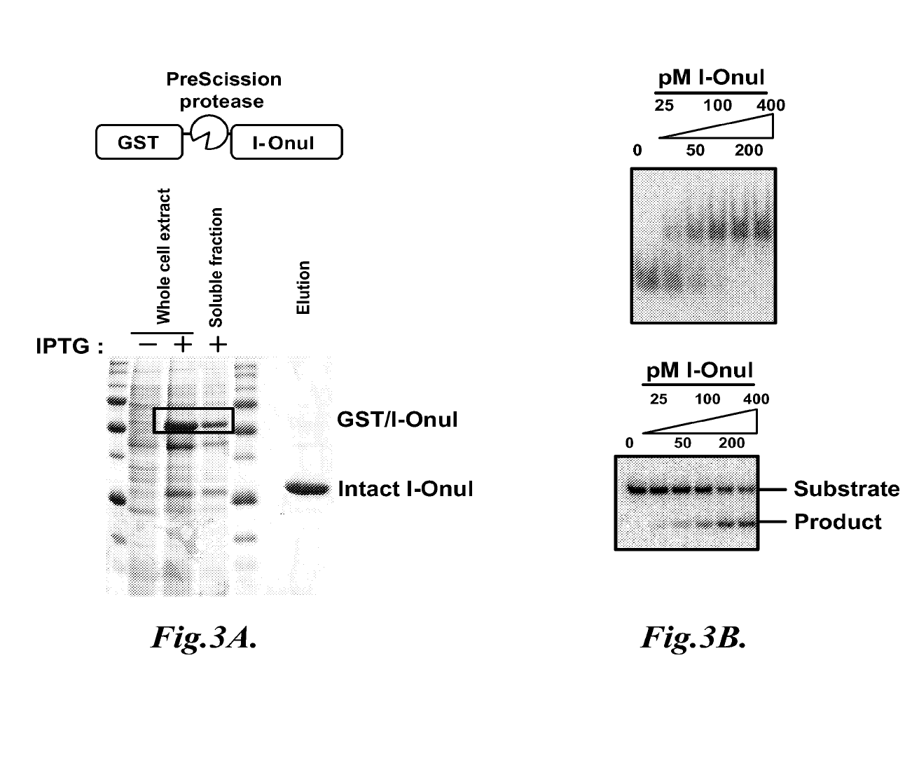

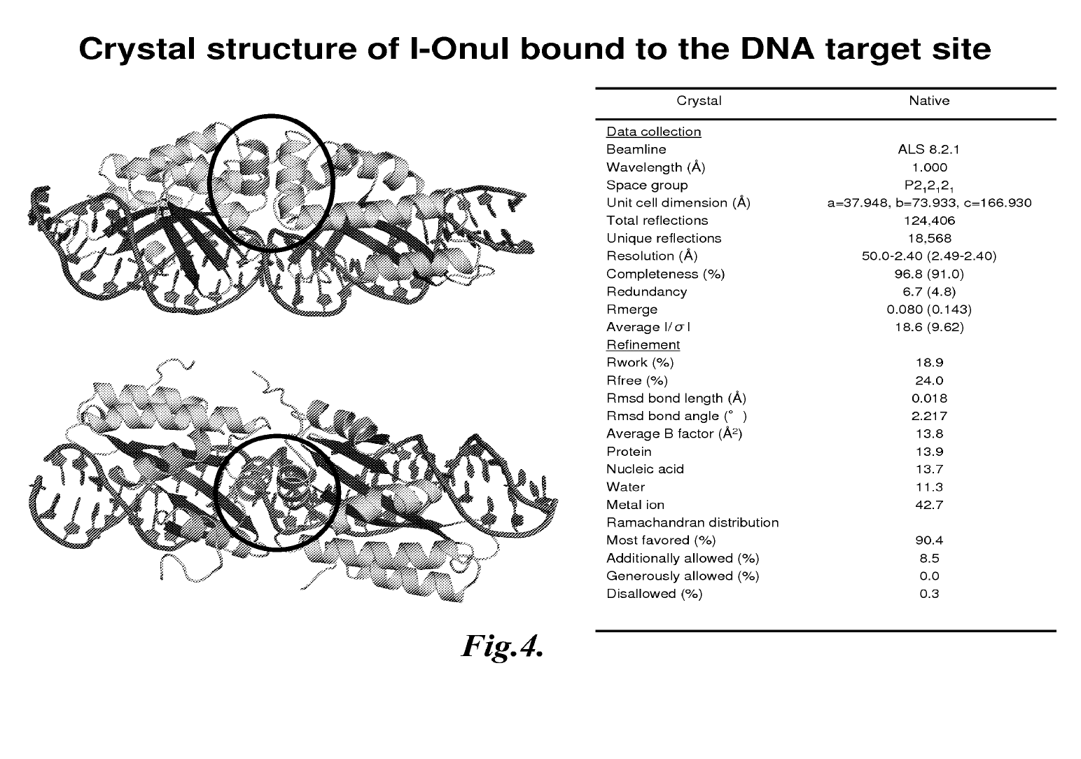

An endonuclease encoded within a group I intron in the RPS3 host gene from Ophiostoma novo-ulmi was identified as potentially displaying the characteristics of an LAGLIDADG homing endonuclease required for gene targeting and subsequent engineerability. The protein (SEQ ID NO: 35) was a monomer and was found in a mesophilic fungal host. The open reading frame of I-OnuI was inserted between BamHI and NotI sites of a pGEX-6p 3expression vector (GE Healthcare Life Sciences), and the GST fusion recombinant proteins were expressed in E. coli strain BL21-CodonPlus (DE3)-RIL (Agilent Technologies). Protein expression was induced in LB medium supplemented with 0.2% glucose, 1 mM MgSO₄, and 100 μg/ml ampicillin at 16° C. for about 20 hours after the culture had achieved early log growth phase (OD600=˜0.6). The harvested cells were resuspended in TDG buffer (20 mM Tris-HCl (pH 7.5), 1 mM dithiothreitol (DTT), and 5% glycerol) supplemented with 0.5 M NaCl. After adding lysozyme to 0.5 mg/ml, the cells were sonicated for 30 sec 6 times, and stirred on ice for 30 minutes. The clarified cell lysate was obtained by centrifugation at 25,000×g for 30 minutes at 4° C., and nucleic acids were precipitated by adding polyethylenimine (pH 7.9) to 0.25% (v/v). After centrifugation at 25,000×g for 10 minutes at 4° C., the supernatant was filtered through a 0.45 μm PVDF membrane, and mixed with glutathione sepharose 4B beads (GE Healthcare Life Sciences). The beads were extensively washed with TDG buffer supplemented with 2 M NaCl, and equilibrated with Digestion buffer (50 mM Tris-HCl (pH 7.0), 0.5 M NaCl, 1 mM dithiothreitol (DTT), and 5% glycerol. The intact I-OnuI and subsequent variant proteins were eluted by incubation with a GST tag specific protease (PreScission® protease (GE Healthcare Life Sciences) for 16 hours at 4° C. The collected proteins were concentrated and stored at −80° C. until use as a GST fusion protein, and subsequently purified by affinity chromatography (FIG. 3). The protein was assayed for binding and cleavage of its putative target site (corresponding to the intron insertion site in the RP3 gene of its biological genomic host) and found to display robust cleavage activity with an approximate dissociation constant (K_{d}) of 3 pM. To assay for the binding of DNA target site by I-OnuI each reaction mixture contained 20 mM Tris-acetate (pH 7.5), 40 mM NaCl, 1 mM CaCl₂, 1 mM DTT, 0.2 mg/ml BSA, 5% glycerol, I-OnuI, unlabeled T7 terminator primer and 10 pM radiolabeled DNA substrate containing the I-OnuI target sequence. After the reaction mixtures were incubated on ice for 10 minutes, the protein-DNA complexes were separated on a 5% polyacrylamide gel containing 20 mM Tris-acetate (pH 7.5) and 1 mM CaCl₂. Gel images were taken with an imager (Typhoon Trio Multi-mode imager (GE Healthcare Life Sciences)). The DNA bands were quantified using the software application ImageJ®, and the dissociation constant (KD) values were calculated using GraphPad® Prism 5 software.

Cleavage of the DNA target site by I-OnuI was determined as follows. Each reaction mixture contained 20 mM Tris-acetate (pH 7.5), 140 mM potassium glutamate (pH 7.5), 10 mM NaCl, 1 mM MgCl₂, 1 mM DTT, 0.2 mg/ml BSA, I-OnuI or the variants, and 10 pM of the radiolabeled substrate used for the electrophoresis mobility shift assay. The reactions ran at 37° C. for 30 min, and were terminated by adding 4× Stop solution (40 mM Tris-HCl (pH 7.5), 40 mM EDTA, 0.4% SDS, 10% glycerol, 0.1% bromophenol blue and 0.4 mg/ml proteinase K). After incubation at 37° C. for 15 minutes, each sample was loaded on a 20% polyacrylamide-TBE gel. The gel images were taken and the DNA bands were quantified as described above.

The purified enzyme was crystallized in complex with its target site, and the structure of the protein-DNA complex was determined to 2.4 Å resolution (FIG. 4). The structure demonstrated that the enzyme forms a monomeric LAGLIDADG (LHE) protein fold with two pseudo-symmetric DNA-binding domains (LD1 amino acid residues 13 to 24 of SEQ ID NO: 35 and amino acid residues 54 to 66 of the consensus sequence shown in FIG. 11; LD2 amino acid residues 160 to 171 of SEQ ID NO: 35 and amino acid residues 238 to 249 in FIG. 11), connected by a flexible linker (amino acid residues 141 to 156 of SEQ ID NO: 35 and amino acid residues 202 to 234 of FIG. 11). The LHE domains form an elongated protein fold that consists of a core fold with mixed α/β topology (α-β-β-α-β-β-α). The overall shape of this domain is a half-cylindrical “saddle” that averages approximately 25 Å×25 Å×35 Å. The surface of the saddle is formed by an anti-parallel β-sheet within each protein domain that presents a large number of exposed basic and polar residues for DNA contacts and binding. The complete DNA-binding surfaces of the full-length enzymes, generated by two-fold pseudo-symmetry, was approximately 80 Å long and accommodated the DNA target site.

The LAGLIDADG motifs of I-OnuI form the last two turns of the N-terminal helices in each folded domain or monomer that are packed against one another. They also contribute N-terminal, conserved acidic residues (E22 and E178) to two active sites where they coordinate divalent cations that are essential for catalytic activity. The structure and packing of the parallel, two-helix bundle in the domain interface of the LAGLIDADG enzymes are strongly conserved among the diverged members of this enzyme family.

### The Recognition of a DNA Target Sequence by I-OnuI.

Amino acid side chains from the two structurally independent, antiparallel β-sheets (one from each protein domain) are used to contact nucleotide bases within the major groove, at positions flanking the central four base pairs as shown in FIG. 5. The 22 basepair target site, as shown in the illustration, is recognized by a combination of at least 22 direct contacts between amino acid side chains and individual DNA bases, at least 14 additional water-mediated contacts between amino acid side chains and individual DNA bases, and at least 30 contacts between the protein and the DNA phosphoribose backbone (mostly water-mediated). At least 40 amino acid residues are involved in direct or water-mediated contacts to the DNA target; these represent the first shell of amino acid residues that can be exploited for the redesign or selection, as described below. The roles of these amino acids are not mutually exclusive: the same side chain can be used for different forms of readout of adjacent DNA bases or backbone atoms.

### The DNA Binding and Cleavage Specificity Profile of I-OnuI.

The DNA binding and DNA cleavage specificity profile of surface-displayed I-OnuI was determined as described in Jarjour et al. (Nucl. Acids Res. 37:6871-6880, 2009, incorporated herein by reference in its entirety) (FIG. 6). The cleavage specificity profile of I-OnuI using a yeast surface display cleavage assay. The cleavage specificity profile of I-OnuI was obtained by measuring the relative cleavage activities of 66 target sequences, each containing a single base-pair substitution from the enzyme's original target site. Cleavage activity against each DNA target was measured using yeast surface-displayed enzyme and flow-cytometry-based tethered DNA cleavage assays. Approximately 100,000 cells expressing I-OnuI were stained with 1:250 dilution biotinylated antibody against hemagglutinin (HA)-epitope tag (Covance) and 1:100 fluorescin isothiocyanate (FITC)-conjugated αMyc (ICL Labs) for 30 minutes at 4° C. in 10 mM Hepes (pH 7.5), 180 mM KCl, 10 mM NaCl, 0.2% BSA, and 0.1% galactose. The cells were then stained with pre-conjugated streptavidin-PE:Biotin-dsOligo-A467 in the same buffer supplemented with 400 mM KCl. The cells were washed in the buffer containing 180 mM KCl, and split into two wells. Each well was then resuspended in the same buffer supplemented with 2 mM of either MgCl₂ or CaCl₂. After incubation at 37° C., the cells were pelleted and resuspended in the buffer containing 400 mM KCl and 4 mM EDTA to enhance release of the cleaved substrates, and analyzed on a BD LSRII cytometer. The relative cleavage activities for each DNA target site were evaluated by calculating the ratio of the non-cleavage median DNA-Alexa647 fluorescence intensity to the post-cleavage intensity in the matching gate.

The binding specificity profile of I-OnuI was determined using a yeast surface display binding assay. The binding specificity profile of I-OnuI was obtained by measuring the relative binding of 66 target sequences, each containing a single base-pair substitution from the enzyme's original target site. The relative binding of each DNA target was measured using yeast surface-displayed enzyme in and flow-cytometry-based untethered DNA retention binding assays. Approximately 100,000 cells expressing I-OnuI were stained with a serial titration of pre-conjugated streptavidin-PE:Biotin-dsOligo-A467 in 10 mM Hepes (pH 7.5), 180 mM KCl, 10 mM NaCl, 0.2% BSA, and 0.1% galactose and 2 mM CaCl₂. After incubation at 37° C., the cells were washed in the same buffer containing 180 mM KCl and the retained, bound DNA was measured by direct fluorescence of the A467 label on the DNA.

The specificity profiles illustrate the ability of the enzyme to bind and cleave a series of alternative DNA target sequences that each contain a single basepair mismatch at each of the 22 positions in the DNA recognition site. The analysis indicates that the majority of specificity of DNA recognition is accomplished during DNA binding, rather than at the chemical step of DNA hydrolysis (although there are several individual basepair substitutions that do not affect binding, but inhibit subsequent cleavage). Those basepair substitutions that can be bound and cleaved as well (or at least with an efficiency greater than 50% that of the corresponding wild-type DNA base) include −6C, −4C, +4T and +4C. The overall specificity of the enzyme is therefore extremely high (at least 1 in 10¹⁰).

Design and Selection of I-OnuI, I-OnuI Homologues and Hybrids Thereof with Altered DNA Binding Specificity at Individual Basepairs of its Naturally Occurring Target Site.

Variants of I-OnuI, including variants of I-OnuI and homologues and hybrids thereof, that can cleave altered DNA target sites (containing individual basepair substitutions) were identified using a combination of structure-based design and genetic selection for cleavage activity. The method relied upon identification of amino acid contact residues near each altered DNA basepair that were identified in the protein-DNA crystal structure (illustrated in FIG. 5) and subsequent selection of surrounding amino acid substitutions (corresponding to a ‘pocket’ of protein side chains that surround the immediate contacting residue at each position in the protein-DNA interface). The analysis identified alternative amino acid identities that can form contacts to altered basepairs at each position in the DNA target, while modeling a conservatively flexible nucleotide and protein backbone. Subsequent creation of limited protein mutation libraries and screening these libraries for DNA cleavage that leads to elimination of a bacterial reporter gene has produced active enzyme variants with desired specificities for many single base-pair substitutions in the wild-type target site of I-OnuI homing endonuclease (FIG. 7, bold amino acid residues). Specifically, desired I-OnuI sequences were mapped on to the I-OnuI-DNA crystal structure and the Rosetta computational design methodology was used to optimize the amino acid sequence of the protein to maximize affinity for the new site. The predicted specificity of the resulting protein models for the desired target sequence was computed using Rosetta, and designs that were predicted to bind tightly and specifically were subjected to further optimization using flexible backbone protein design. The tightest binding and most specific designs were again selected, and the designed amino acid substitutions were removed one at a time. If no significant loss was predicted in either specificity or binding energy, the substitution was removed from the design. Genes encoding the designed proteins were assembled from oligonucleotides, and the designed proteins were expressed, purified, and assayed as described above.

The selection system was also modified to allow for selection of enzyme specificity as well as activity by selecting against enzyme variants that can still cleave the wild-type target site. In one particular embodiment, the open reading frames of I-OnuI was inserted between NcoI and NotI sites of the pEndo expression vector. Expression of the I-OnuI gene was tightly regulated by the pBAD promoter, and addition of L-arabinose promoted gene transcription. Site-directed mutagenesis or random mutagenesis on I-OnuI gene was induced both by overlap extension PCR, and by using GeneMorph II Random Mutagenesis Kit (Agilent Technologies), by following the manufacture's instructions. Two copies each of a desired I-OnuI target site were inserted between AflIII and BglII and between NheI and SacII of the reporter “pCcdB” plasmid. This plasmid encodes “control of cell death B” protein, which is toxic to bacteria; the expression of this gene is induced by isopropyl-β-thiogalactopyranoside (IPTG). The cleavage of the target sites by the expressed LHEs leads to the RecBCD-mediated degradation of the reporter plasmid, and rescues the cell growth on the selective medium containing IPTG. The pEndo plasmid was transformed into NovaXGF′ (Novagen) competent cells harboring pCcdB plasmid (containing four copies of the I-OnuI or MAO-B target) by electroporation. The transformants were grown in 2×YT medium at 37° C. for 30 minutes, and were 10-fold diluted with 2×YT medium supplemented with 100 mg/ml carbenicillin and 0.02% L-arabinose. After the culture was grown at 30° C. for 4-15 hours, the cells were harvested, resuspended in sterilized water, and spread on both non-selective plates (1×M9 salt, 1% glycerol, 0.8% tryptone, 1 mM MgSO₄, 1 mM CaCl₂, 2 mg/ml thiamine, and 100 mg/ml carbenicillin) and selective plates (the non-selective plates supplemented with 0.02% L-arabinose and 0.4 mM IPTG). For negative selection to remove the variants active against the wild type target sequence, the transformants were spread on the selective plates containing 33 mg/ml chloramphenicol instead of IPTG. The plates were incubated at 30° C. for about 30 to 40 hours. To proceed to the next round of selection, the pEndo plasmid was extracted from the surviving colonies on the selective plates. The ORFs of I-OnuI variants were recovered by PCR amplification, and digested with NcoI, NotI, and ScaI or PvuI. The resulting fragments were cloned into pEndo vector, and subjected to further selection.

### Engineering of a Variant of I-OnuI Against a Physiological Target Site in the Human Genome (the Monoamine Oxidase B (MAO-B) Gene).

A variant of the I-OnuI endonuclease has been engineered that targets a unique sequence in the MAO-B target site, and that can be used to direct homologous recombination in that gene locus, leading to targeted point mutations of the endogenous MAO-B gene and development of engineered neural cell lines for in vivo studies.

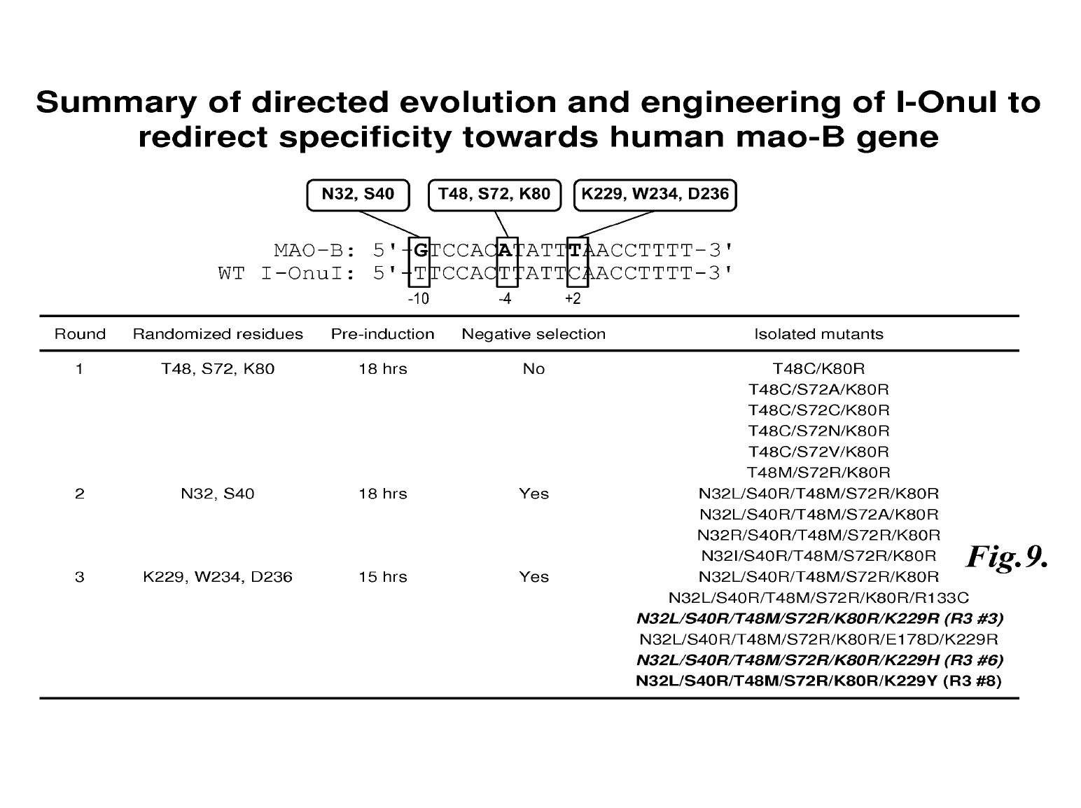

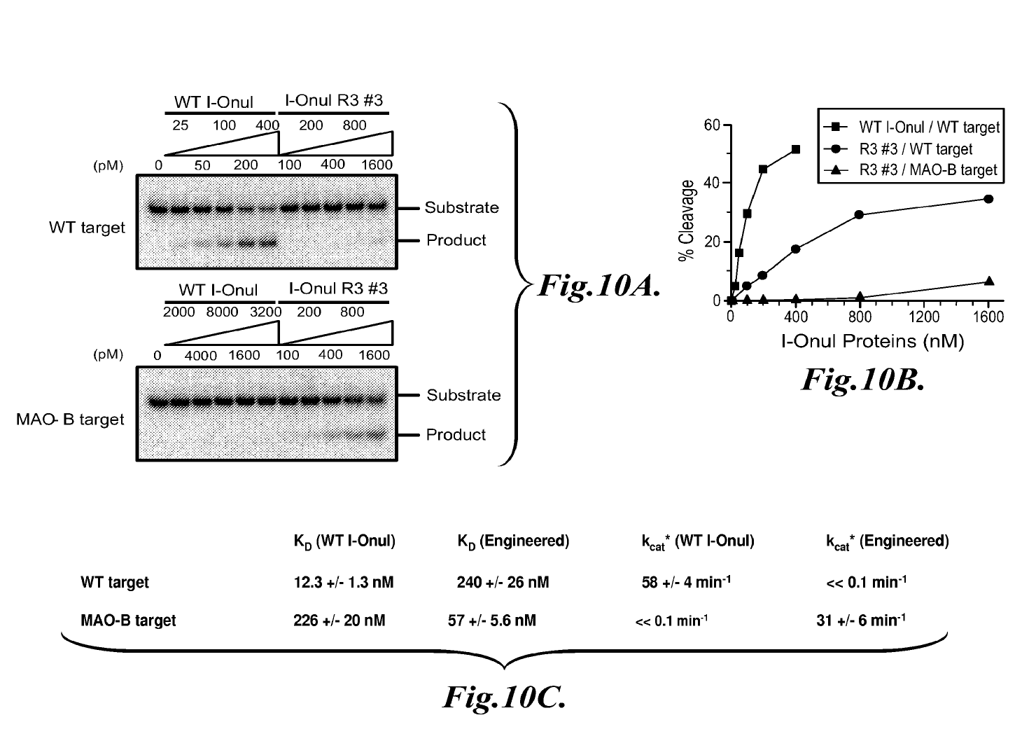

Design and selection of the MAO-B specific variant of I-OnuI was carried out by identification and mutation of residues in the protein −DNA interface that contact individual basepairs that differ between the desired human target site (MAO-B) and the wild-type I-OnuI recognition site (FIG. 9). The mutation and redesign process was carried out in a sequential, iterative manner, by isolating enzyme mutations that could accommodate and cleave DNA variants with basepair substitutions corresponding to −10G, −4A and +2T, respectively. Directed evolution of I-OnuI endonuclease to cleave the human MAO-B gene target was carried out using the in vivo bacterial screening methodology described for FIG. 7, and a scheme for mutagenesis in which all residues within 5 angstrom contact distance of the altered base pairs in the target site (N32, S40, T48, S72, K80, K229, W234, and D236) were randomized, and selections were carried out in sequential order of addition as shown in rounds 1, 2 and 3 of the figure. Addition of an E178D substitution into the active site of E1 I-OnuI increased cleavage activity of the enzyme and was included in the final redesigned enzyme construct (E178 is one of two residues that coordinate divalent metal ions in the active site. Electrophoretic mobility shift assays using purified recombinant proteins, as described above, demonstrated that wild-type I-OnuI preferentially bound its physiological target with a very tight dissociation constant (193±15.2 pM). The E1 and E2 I-OnuI proteins displayed similar affinity for both the WT and MAO-B targets; however these enzymes significantly discriminated between the two target sites in cleavage reactions. The relative cleavage activities assayed in vitro correlated well with the GFP gene conversion frequencies that were measured using the DR-GFP reporter. For instance, E2 I-OnuI induced GFP gene conversion on the MAO-B target approximately 3-fold more efficiently than E1 I-OnuI, and displayed a very similar level of in vitro cleavage activity for the MAO-B target at approximately 4 fold lower enzyme concentrations. The final variants of I-OnuI that displayed altered cleavage specificity towards the desired human MAO-B target site (denoted R3 #3, R3#6 and R3#8 in FIG. 9) each harbored six amino acid substitutions: N32L, S40R, T48M, S27R, K80R, and K229R, H, or Y). The relative binding and cleavage activity of wild-type and “R3 #3” variant of I-OnuI towards the wild-type and human MOA-B genomic target site were assayed (FIG. 10). The relative binding by wild-type (WT) and redesigned (R3#3) I-OnuI towards the original (WT) and MAO-B DNA target sites were performed using electrophoretic mobility shift assays and the relative cleavage of by wild-type (WT) and redesigned (R3#3) I-OnuI towards the original (WT) and MAO-B DNA target sites were performed using in vitro enzyme cleavage digests as described above. In addition, the relative cleavage of by wild-type (WT) and redesigned (R3#3) I-OnuI towards the original (WT) and MAO-B DNA target sites were performed using in vitro enzyme cleavage digests as described above. Sequences of human MAO-B gene targets before and after treatment with redesigned I-OnuI enzyme were determined by extracting genomic DNA from sorted cells (˜1×10⁵) which had been washed with cold PBS buffer, resuspended in TNES buffer (50 mM Tris-HCl (pH 8.0), 150 mM NaCl, 10 mM EDTA, 1% SDS, 0.25 mg/ml proteinase K), and incubated at 50° C. for 30 minutes. RNase A was added to 0.25 mg/ml, and the reaction mixture was further incubated at the same temperature for 30 minutes. The genomic DNA was recovered by phenol/chloroform/isoamyl alcohol (PCI) extraction followed by ethanol precipitation. Both of the on-target (i.e., MAO-B gene) and off-target loci were amplified from 50-80 ng of the extracted genomes using Phusion DNA polymerase (Finnzymes). The DNA products resulting from 2 rounds of PCR amplification were cleaned using a PCR purification kit (Qiagen). Individual clones were sequenced using dye-terminator Sanger sequencing on an ABI automated DNA sequencer

In addition, small deletions were found within the endogenous MAO-B target site during targeted mutagenesis of the endogenous MAO-B gene in human tissue culture cells. Table 1. The intact genome sequence of the MAO-B gene, prior to treatment with the R3 #3 variant of I-OnuI is shown at the top of the Table and the MAO-B target sequence is underlined; the mutated sequences of the same gene caused by treatment with the R3 #3 variant of I-OnuI are aligned below.

TABLE 1
Deletions found in the endogenous MAO-B gene using the R3#3 variant I-OnuI for targeted mutagenesis.

SEQ ID NO: 110 CTGGGTTGGTCCAACATAGGATCCTCCAAGGTCCACATATTTAACCTTTTGGTTCTGTTTTCCCATAGGAAAAAATTAAA
SEQ ID NO: 111 CTGGGTTGGTCCAACATAGGATCCTCCAAGGTCCACAT-TTTAACCTTTTGGTTCTGTTTTCCCATAGGAAAAAATTAAA
SEQ ID NO: 112 CTGGGTTGGTCCAACATAGGATCCTCCAAGGTCCACATATTT--CCTTTTGGTTCTGTTTTCCCATAGGAAAAAATTAAA
SEQ ID NO: 113 CTGGGTTGGTCCAACATAGGATCCTCCAAGGTCCACA-----AACCTTTTGGTTCTGTTTTCCCATAGGAAAAAATTAAA
SEQ ID NO: 114 CTGGGTTGGTCCAACATAGGATCCTCCAAGGTCCACATA--------TTTGGTTCTGTTTTCCCATAGGAAAAAATTAAA
SEQ ID NO: 115 CTGGGTTGGTCCAACATAGGATCCTCCAA---------ATTTAACCTTTTGGTTCTGTTTTCCCATAGGAAAAAATTAAA
SEQ ID NO: 116 CTGGGTTGGTCCAACATAGGATCCTCCAAGGTCCA---------CCTTTTGGTTCTGTTTTCCCATAGGAAAAAATTAAA
SEQ ID NO: 117 CTGGGTTGGTCCAACATAGGATCCTCCAAGGTCCACATATTTA--------------TTTTCCCATAGGAAAAAATTAAA
SEQ ID NO: 118 CTGGGTTGGTCCAACATAGGATCCTCCAA---------------CCTTTTGGTTCTGTTTTCCCATAGGAAAAAATTAAA

### Methods for Knockout of the MAO-B Gene

A plasmid construct to express engineered I-OnuI (pExodus®) was created in which the gene including the N-terminal hemagglutinin (HA) tag, followed by a nuclear localization signal was linked to an mCherry gene by the 2A peptide sequence from Thosea asigna virus (T2A). The two-gene expression was driven by a cytomegalovirus (CMV) promoter, and the co-translated proteins were separated by ribosomal skipping. The DR-GFP reporter codes a GFP gene sequence interrupted by a HE target site and an in-frame stop codon, followed by the truncated gene sequence. Human embryonic kidney (HEK) 293T cells were grown in Dulbecco's modified Eagle medium (DMEM) supplemented with 10% fetal bovine serum, 10 units/ml penicillin and 10 mg/ml streptomycin at 37° C. in 5% CO₂ atmosphere. 6×10⁴ of HEK 293T cells were plated 24 h prior to transfection in 12-well plates, and transfected with 0.25 mg each of DR-GFP reporter and pExodus® plasmid using a transfection reagent (Fugene® 6 transfection reagent (Roche Applied Science)). The GFP positive cells were detected by flow cytometry at 48 h post transfection. Western blotting was carried out using rabbit polyclonal antibody against hemagglutinin (HA)-epitope tag and mouse monoclonal antibody against β-actin.

HEK 293T cells (1.3×10⁵) were plated 24 hours prior to transfection in 6-well plates, and transfected with 1 mg of pExodus® plasmid. The top 25% and the following 25% of mCherry positive cells (fluorescent marker for a LHE gene expression) were separately collected using BD FACSAria® cell sorter (BD Biosciences) 48 hours post transfection. To extract genomic DNA, the sorted cells (˜1×10⁵) were washed with cold PBS buffer, resuspended in TNES buffer (50 mM Tris-HCl (pH 8.0), 150 mM NaCl, 10 mM EDTA, 1% SDS, 0.25 mg/ml proteinase K), and incubated at 50° C. for 30 minutes. RNase A was added to 0.25 mg/ml, and the reaction mixture was further incubated at the same temperature for 30 minutes. The genomic DNA was recovered by phenol/chloroform/isoamyl alcohol (PCI) extraction followed by ethanol precipitation. Both of the on-target (i.e., the MAO-B gene) and off-target loci were amplified from 50-80 ng of the extracted genomes using a DNA polymerase (Phusion® DNA polymerase, Finnzymes). The DNA products resulting from 2 rounds of PCR amplification were cleaned using a PCR purification kit (Qiagen), and 150 mg of the fragments were incubated with 1.5-3.0 pmol of E2 I-OnuI recombinant protein in 20 mM Tris-acetate (pH 7.5), 100 mM potassium acetate (pH 7.5), 1 mM DTT and 10 mM MgCl₂ at 37° C. for 2 hours. The cleavage reactions were terminated by adding 4× Stop solution (40 mM Tris-HCl (pH7.5), 40 mM EDTA, 0.4% SDS, 10% glycerol, 0.1% bromophenol blue and 0.4 mg/ml proteinase K). After incubation at 37° C. for 30 minutes, each sample was separated on a 1.8% agarose gel containing ethidium bromide in TBE. The DNA bands were quantified using ImageJ® software. The MAO-B gene was successfully knocked out using this method.

### The Identification and Modeling of Structural and Functional Homologues of I-OnuI.

Recent high-throughput sequencing of Ascomycete fungi have resulted in the discovery of unique lineages of LAGLIDADG homing endonucleases inserted in a variety of host genes. (Sethuraman et al., Mol. Biol. Evol. 26:2299-2315, 2009). These endonucleases display a wide repertoire of new specificities. At least 26 recognizable homologues of I-OnuI, displaying an average of 45% sequence identity, similar overall peptide chain lengths, and at least 50% sequence identity of amino acid sequence within the two LAGLIDADG domains and the “Loop sequence” as described above, relative to that enzyme, have been identified and aligned (FIG. 11). Using this data and the crystal structure of I-OnuI, structure-based homology models of these homing endonucleases have been generated with a corresponding list of predicted DNA-contacting residues for each enzyme.

### Methods for Selection of Homologues and Alignment to I-OnuI

Putative full-length LAGLIDADG homing endonuclease that displayed homology to the I-OnuI homing endonuclease were collected from the Pfam database, available on the world-wide-web and described in Finn et al., Nucl. Acid Res. 38:D211-D222, 2010, based on an observed value at least 40% amino acid sequence identity to the I-OnuI protein. Structure-based multiple sequence alignments were then built using Cn3D application available at the National Center for Biotechnology Information website. After the structure of I-OnuI was aligned to the structure of I-Anil (PDB 1P8K) the collected Pfam sequences were aligned to the I-OnuI and I-Anil structures, at which point homodimeric LAGLIDADG sequences were removed based on length and the occurrence of a single LAGLIDADG motif per sequence. The sequence alignment generated by the Cn3D application was subsequently validated using a modified version of the Java based multiple alignment editor application Jalview that calculates the MIp/Zp co-variation statistic in real-time while the alignment is edited. Groups of misaligned sequences were realigned to minimize local co-variation, as local co-variation is a unique indicator of misalignment that is independent of methods used to build the multiple sequence alignment. Local co-variation was also used as a guide to reject partial and erroneous sequences.

### Methods for Solving the Crystal Structure of I-LtrI

To express and purify I-LtrI, 10 ml of an E. coli culture containing a pET plasmid with the I-LtrI endonuclease (pET-15HE-Ltr) was grown overnight and diluted 1:100 into 1 liter of Luria-Bertani media. The 1 liter culture was grown at 37° C. for 3 hours, shifted to 27° C., and expression induced by adding isopropyl-b-D-thiogalactopyranoside to a final concentration of 1 mM. After additional growth for 2.5 hours, cells were harvested by centrifugation at 5000 rpm for 5 min and the pellet was frozen at −80° C. For protein purification, the frozen cells were thawed in the presence of protease inhibitor (Roche Diagnostic) and resuspended in 10 ml of lysis buffer (20 mM Tris-HCl, pH 7.9, 500 mM NaCl, 40 mM imidazole and 10% glycerol) per 1 gm of wet cell weight. Cells were disrupted by homogenization followed by centrifugation at 27,200 g for 25 min at 4° C. The supernatant was sonicated to facilitate DNA fragmentation, and centrifuged at 20,400 g for 15 min at 4° C. The supernatant was applied to a HisTrap HP® Affinity column (GE Healthcare) that had been charged with 0.1 M NiSO₄ and equilibrated with binding buffer (20 mM Tris-HCl, pH7.9, 500 mM NaCl, 40 mM imidazole, and 10% glycerol). Bound protein was eluted with elution buffer (20 mM Tris-HCl, pH7.9, 500 mM NaCl, and 10% glycerol) over a linear gradient of imidazole from 0.08 to 0.5 M, and 0.5 mL fractions were collected over 50 ml. To prevent precipitation, 500 microliters of 2 M NaCl and 10 microliters of 0.5 M EDTA, pH8.0, were added to peak fractions. The peak fraction was loaded directly onto a Superdex 75 gel-filtration column (GE Healthcare) equilibrated with lysis buffer without imidazole. Fractions were collected in 0.25-ml aliquots over 25 ml.

To obtain I-LtrI-DNA co-crystals, the DNA oligonucleotides (5′-GGTCTAAACGTCGTATAGGAGCATTTGG-3′ (SEQ ID NO: 119) and 5′-CAAATGCTCCTATACGACGTTTAGACCC-3′ (SEQ ID NO:120)) were purchased from Integrated DNA Technologies (1 mmole scale, standard desalting purification). The oligonucleotides were dissolved in TE buffer (10 mM Tris-HCl (pH 8.0) and 1 mM EDTA), and the complementary DNA strands were annealed by incubation at 95° C. for 10 min and slow cooling to 4° C. over a six hour period. One hundred mM I-LtrI protein in 50 mM Hepes-NaOH (pH 7.5), 150 mM NaCl, 5 mM MnCl₂ and 5% (v/v) glycerol was mixed with 1.5-fold molar excess of the DNA substrate. The protein-DNA drops were mixed in a 1:1 volume ratio with a reservoir solution containing 100 mM Bis-Tris (pH 6.5), 200 mM magnesium chloride, and 20% (v/v) polyethylene glycol 3500 and equilibrated at 22° C. The crystals diffracted up to approximately 2.7 Å resolution at the ALS beamline 5.0.1. The data set was processed using HKL2000 package. The polyalanine model of I-OnuI/DNA complex (PDB ID: 3QQY) was used as a search model for molecular replacement. One copy of the search model was found and the structure was refined using REFMAC5. The final model was deposited in RCSB Protein Data Bank with ID code 3R7P.

Expression of I-OnuI Homologues; Prediction and Confirmation of their Individual DNA Recognition Sites.

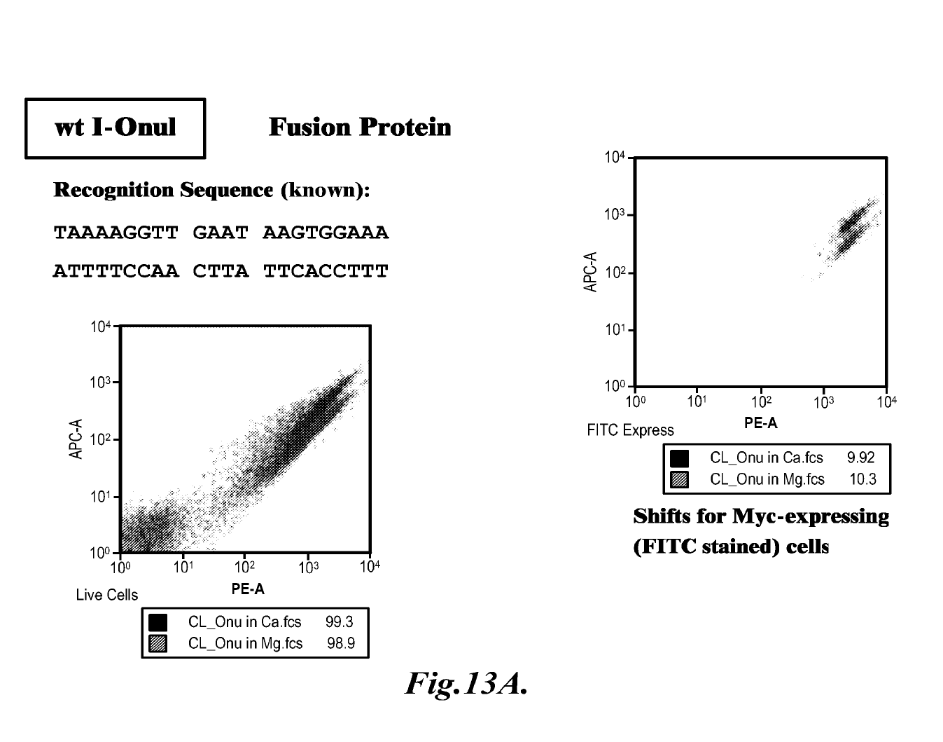

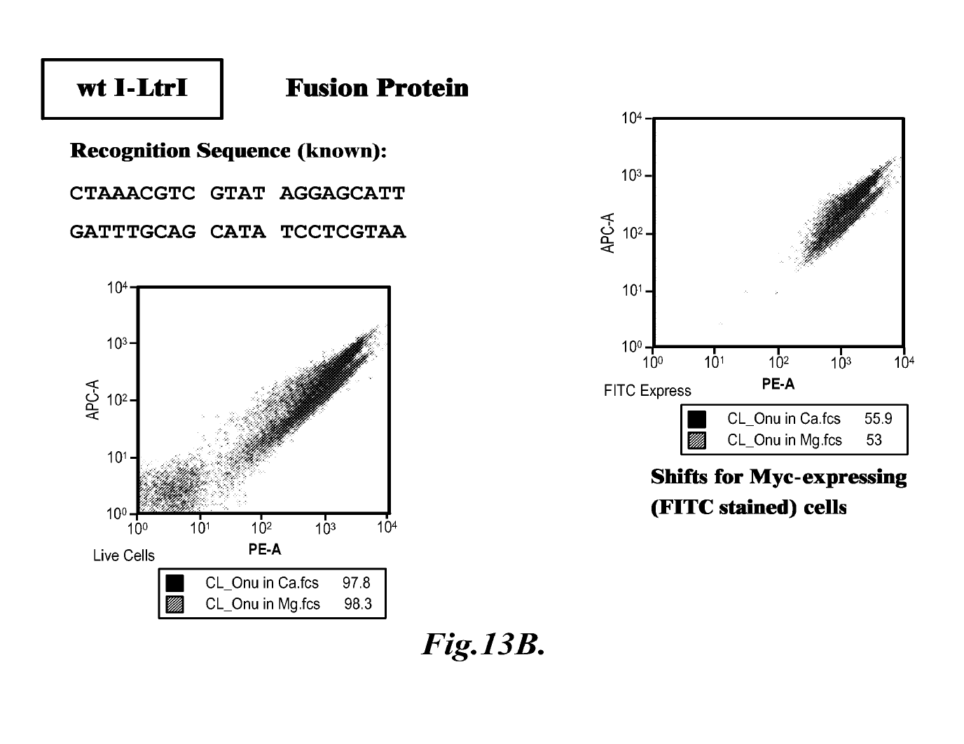

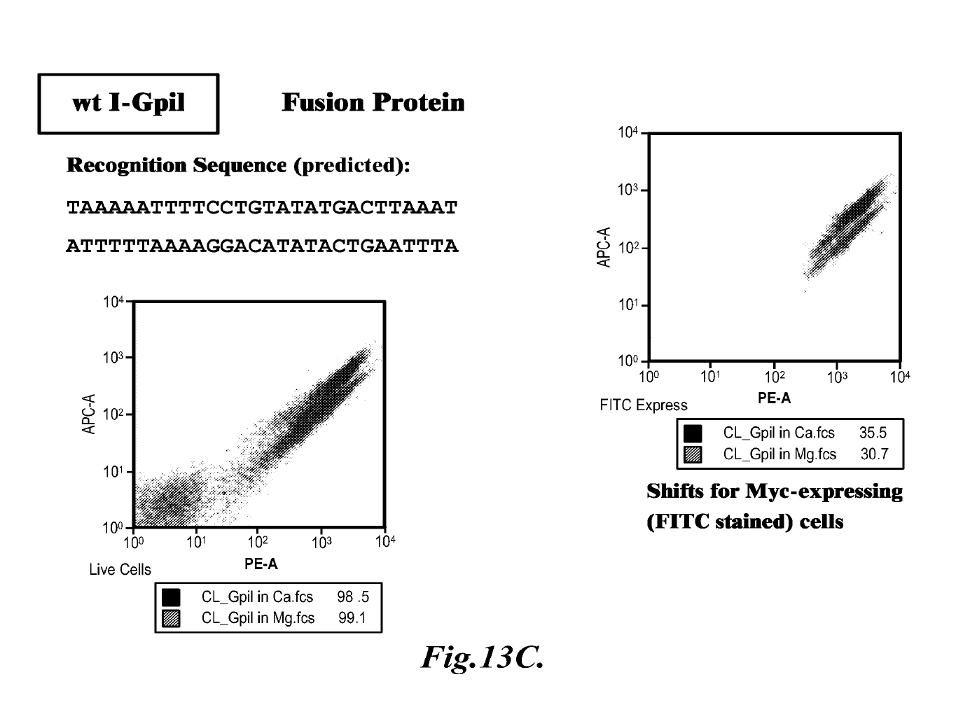

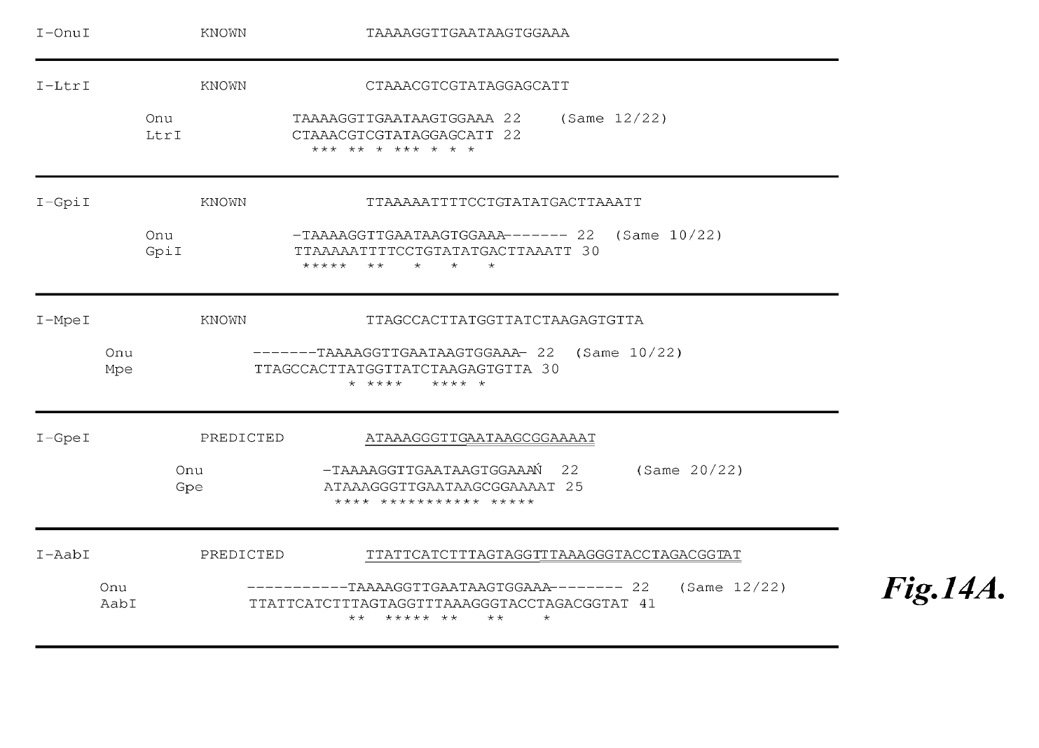

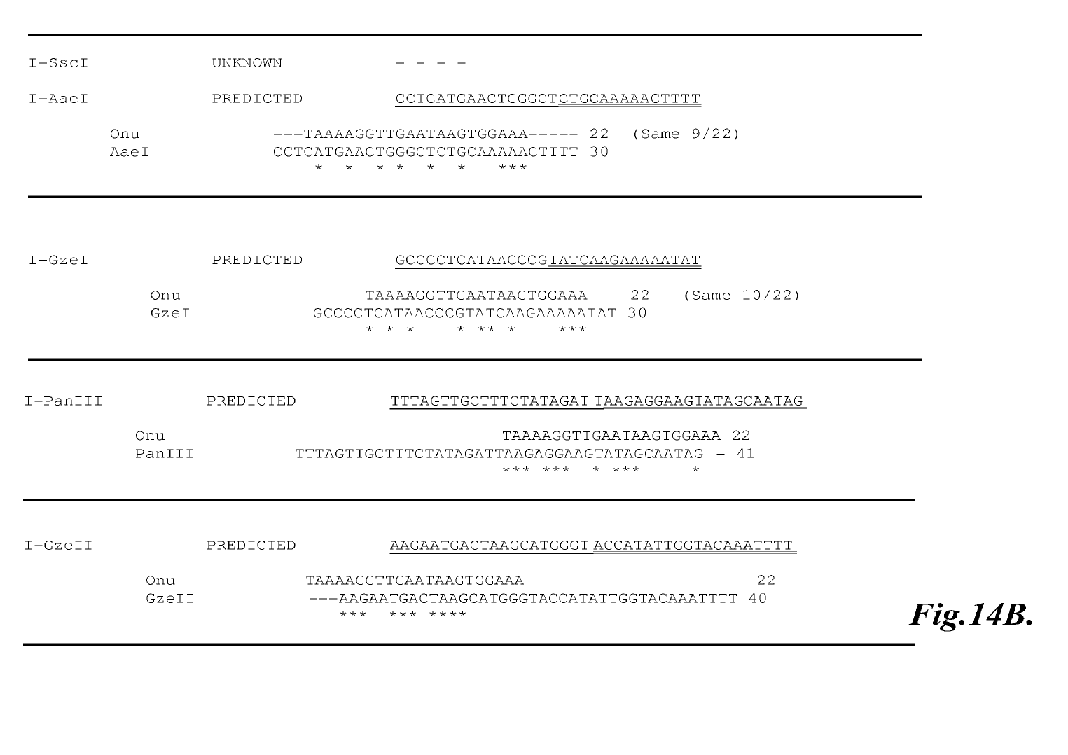

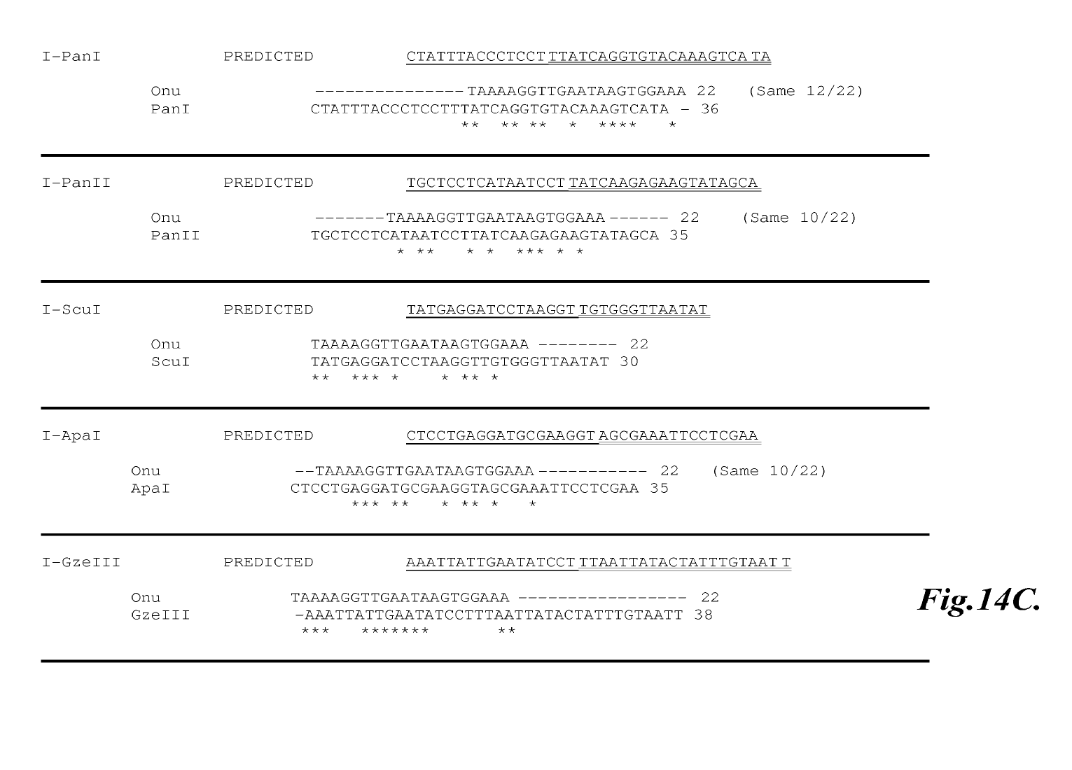

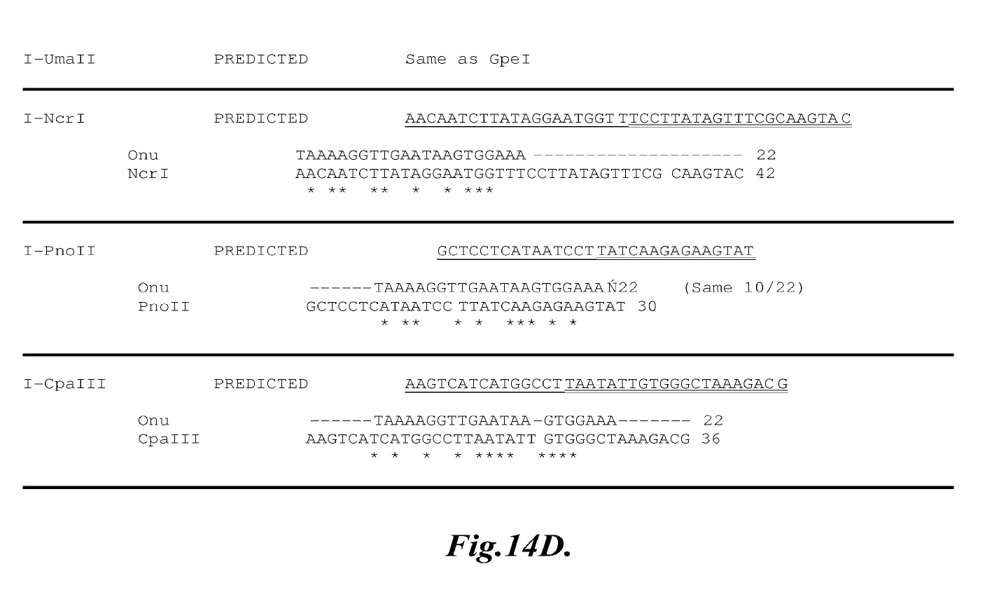

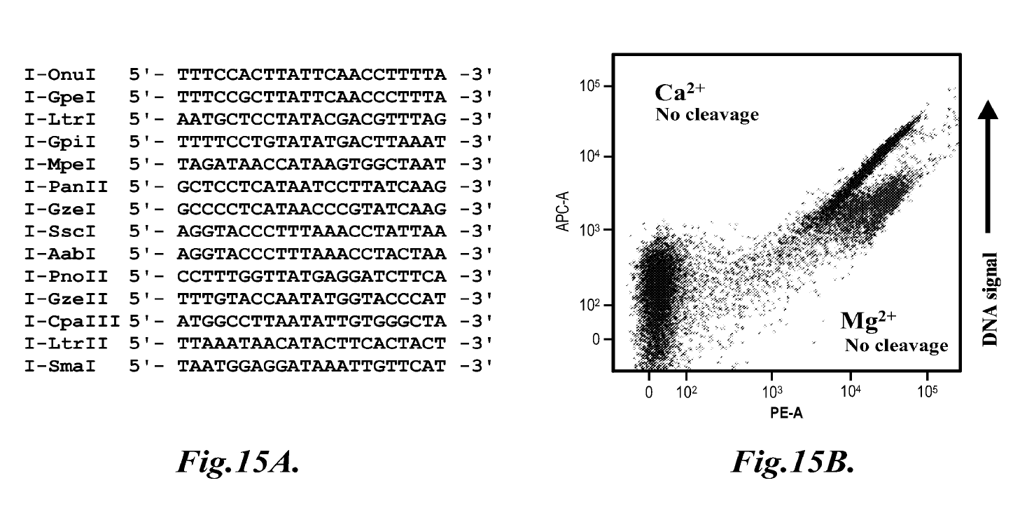

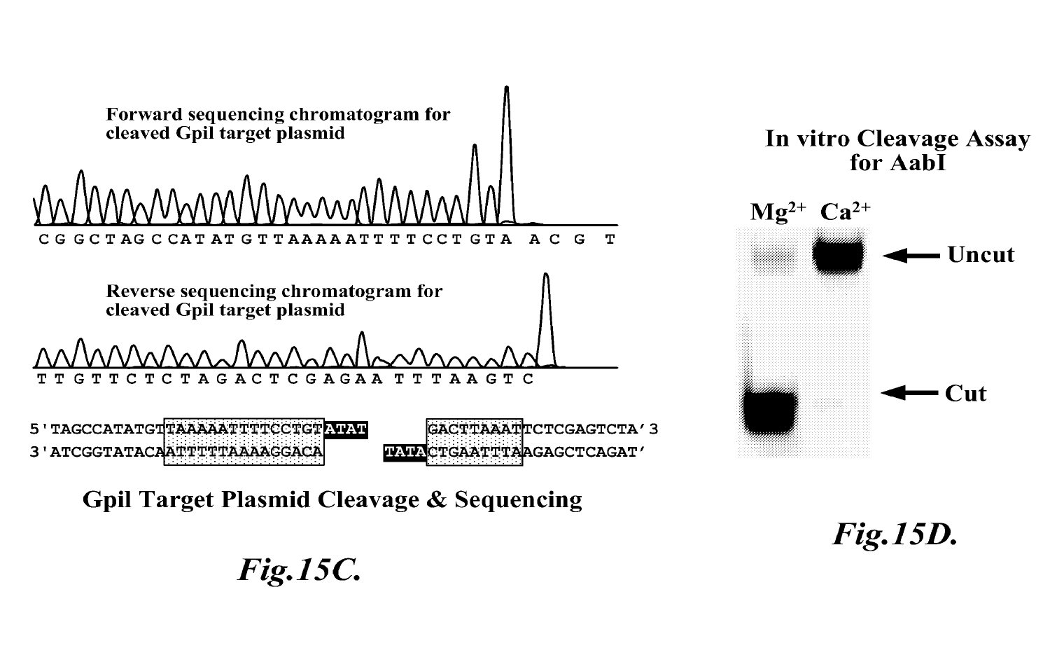

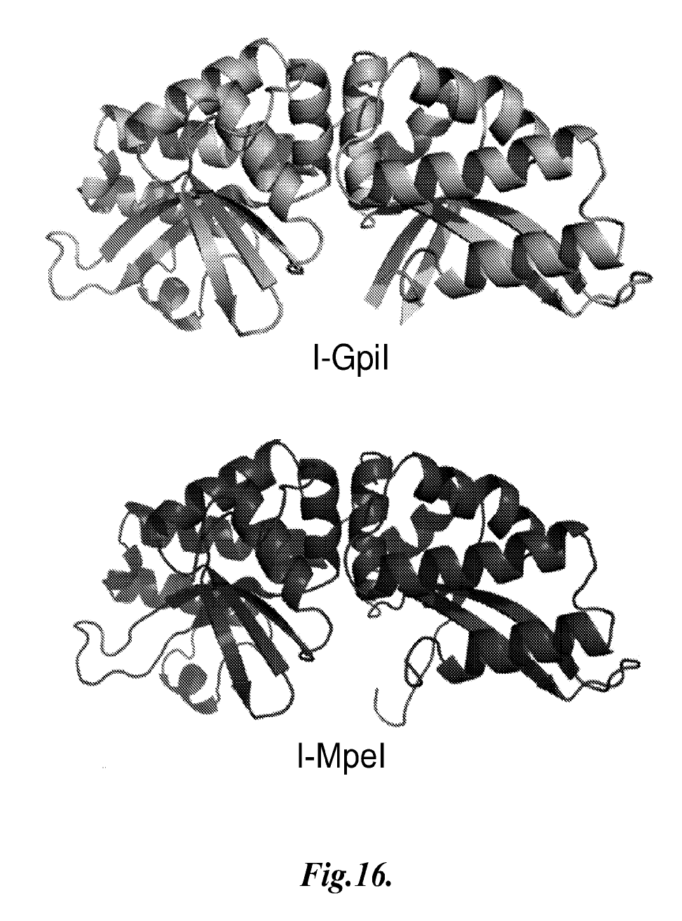

The reading frames corresponding to twelve homologues of I-OnuI have been cloned and used to test expression and cleavage activity on the surface of yeast (FIG. 13). As well, several of the same genes have been placed into inducible bacterial expression system and tested. Of twelve individual clones that have thus far been examined (CpaI, GpiI, GzeI, HjeII, LtrI, MpeI, PanI, PanII, PanIII, PnoII, ScuI, and SscI), six have displayed surface expression on yeast using the methods described in WO 2007/123636, incorporated herein by reference, and production of soluble protein in bacteria (indicating proper folding and formation of stable protein).

Target sites for each LHE were predicted through comparison of the LHE-harboring host gene to related genes lacking an endonuclease. Cleavage activity against each predicted target was verified using yeast surface-displayed enzyme in both in vitro and flow-cytometry-based tethered DNA cleavage assays (7) with the following modifications. Briefly, approximately 5×10⁵ cells expressing a LHE were stained with 1:250 dilution biotinylated antibody against hemagglutinin (HA)-epitope tag (Covance) and 1:100 fluorescin isothiocyanate (FITC)-conjugated αMyc (ICL Labs) for 30 minutes at 4° C. in 10 mM Hepes (pH 7.5), 180 mM KCl, 10 mM NaCl, 0.2% BSA, and 0.1% galactose. The cells were then stained with pre-conjugated streptavidin-PE:Biotin-dsOligo-A467 in the same buffer supplemented with 400 mM KCl. The cells were washed in the buffer containing 180 mM KCl, and split into two wells. Each well was then resuspended in the same buffer supplemented with 2 mM of either MgCl₂ or CaCl₂. After incubation at 37° C., the cells were pelleted and resuspended in the buffer containing 400 mM KCl and 4 mM EDTA to enhance release of the cleaved substrates, and analyzed on a BD LSRII cytometer.

To identify the exact cleavage positions and overhangs that were generated for each target site, the verified sequences were cloned into the pCTCON2-ARL vector (pCTCON2 with modified cloning sites) between the NdeI and XhoI cloning sites. Intact plasmid containing the target sequence was first digested with the HindIII restriction enzyme to create a 6300 bp linear substrate. Cleavage of the target sequence in this linear substrate creates two bands (1.3 kb and 5 kb), which were clearly distinct from the uncut substrate (6.3 kb). Each in vitro cleavage reaction contained five million yeast expressing a LHE on the surface, 10 mM DTT (to release the enzyme from the yeast surface), 5-10 micrograms of HindIII-linearized target plasmid, 5 mM MgCl₂, 10 mM Hepes (pH 7.5), 180 mM KCl, 10 mM NaCl, 0.2% BSA, and 0.1% galactose. After incubation at 37° C. for 2-4 hours, the yeast cells were then spun down and the supernatant was loaded onto an agarose gel. The two product bands were purified from the gel and sequenced, using a forward primer (5′-GTTCCAGACTACGCTCTGCAGG-3′, SEQ ID NO: 121) for the 5 kb band, and a reverse primer (5′-GTGCTGCAAGGCGATTAAGT-3′, SEQ ID NO: 122) for the 1.3 kb band. The sequencing reads ended abruptly at the position of each DNA strand cleaved by the enzyme.

The predicted DNA target sites for each enzyme correspond to DNA sequences encompassing the intron or endonuclease gene insertion site in the host gene and corresponding biological genomes that is the source of the homing endonuclease reading frame (FIG. 13). The sequence identity of these target sites, relative to that of I-OnuI, range from 41% to 91%. The target site of I-OnuI, I-LtrI, I-GpiI, and I-MpeI have been verified using standard in vitro cleavage assays.

### Methods for Homology Modeling

Homology models were created using the SWISS-MODEL automated homology modeling server available on the world-wide-web and described in Arnold et al. Bioinformatics 22:195-201, 2006. Amino acid sequences for each homologue were provided as input, and the structure of either I-OnuI or I-LtrI was designated as the modeling template. Homology models for I-GpiI and I-MpeI are shown in Figure xx.

### Creation of Hybrid LAGLIDADG Homing Endonucleases Via Sequence Substitutions Between I-OnuI Enzyme Homologues.

Homology models of three homologues of I-OnuI (I-LtrI, I-PnoII and I-MpeI) were used to identify surface residue positions which could be mutated to create uniform protein surfaces and increased overall sequence homology between these enzymes. Residues near to the protein-DNA interface were avoided, to maintain the unique DNA-binding preferences of each individual enzyme. Multiple sequence alignments helped guide the choice of amino acid for each surface position, with the ultimate goal being both elimination of hydrophobic residues on the surface, and selection of amino acid sidechains commonly found in those homologs with the best surface expression in yeast and highest solubility and activity in bacteria. These sequence exchanges between homologous enzymes resulted in overall increased DNA coding identity (from initial values of 40% to 50%, up to 70% to 80% between the enzymes tested while maintaining enzyme activity). Therefore, ‘hybrid’ enzymes containing sequence elements from individual members of the I-OnuI family homologues, can be generated and used as a broad set of protein scaffolds for design and selection of additional DNA cleavage specificities.

### Methods for Construction of Hybrid (Chimeric) Homing Endonucleases.

N- and C-terminal half-domains of two I-OnuI homologues (I-OnuI and I-LtrI) were constructed by assembly PCR using oligonucleotides designed by the DNAworks server (Hoover and Lubkowski, Nucl. Acids Res. 30:e43, 2002). Each half-domain construct was flanked by 30-50 base pairs of the pETCON vector to facilitate cloning into that expression vector via homologous recombination. The design for the genes encoding these chimeras included a Ser-Gly-Thr linker between the N- and C-terminal protein domains (which can be encoded by a DNA sequence containing a unique KpnI restriction site, which is useful for subsequent recloning and fusion of new domain combinations). The PCR product for each half-domain was purified using a PCR purification kit (Qiagen), then digested with KpnI (Fermentas) for 15 min at 22° C. Digested N-terminal and C-terminal half-domains were then combinatorially mixed and ligated together to create genes encoding the desired full-length chimeras. In the case of chimeric enzymes lacking the synthetic Ser-Gly-Thr sequence and corresponding KpnI restriction site, the entire full-length enzyme was constructed by PCR assembly.

The genes encoding the full-length hybrid chimeras were combined at approximately 1:10 molar ratio with the yeast-surface display vector pETCON (Genscript), digested with XhoI and NdeI (NEB) for 4 hrs at 37° C. The digested vector and assembled enzymes were transformed into Saccharomyces cerevisiae strain EBY100 using the lithium-acetate protocol (Gietz and Schiestl, Nat. Prot. 2:38-41, 2007). Transformed yeast were grown for 3 days at 30° C. in selection media. Clones were obtained by isolating plasmids from yeast populations using the a plasmid preparation kit (Zymoprep-II® kit; Zymo Research) and electroporating these into Escherichia coli DH10B (Invitrogen) for sequencing. The bacterial population harboring the correct plasmid was then grown overnight, and the clonal plasmid was isolated using a DNA miniprep kit (Qiagen); this plasmid was then transformed into EBY100 yeast by LiAc protocol, as above.

Subsequent to the creation of the initial chimeric endonuclease constructs as described above, the residues that comprise the domain interface in and near the LAGLIDADG motif were randomized, and active constructs were selected. In order to create protein libraries randomized at specified LAGLIDADG interface residues, oligonucleotides containing NNS codons were substituted in the PCR assembly reaction and transformed into EBY100 yeast, as above. Library size was determined by serial dilution, with typical yields of approximately 10×10⁶ unique transformants. Mutation distribution and frequencies were verified by sequencing of an unselected library, and no biases were noted. Yeast were propagated in selective growth media with 2% raffinose +0.1% glucose at 30° C. for 12 to 20 hours, and then induced in media with 2% galactose for 2 to 3 hrs at 30° C., followed by 16 to 24 hrs at 20° C.

Active chimeras were identified and isolated using a flow-cytometric protocol as described in Jarjour et al., Nucl. Acids Res. 37:6871-6880, 2009. Briefly, yeast surface-displayed chimeric homing endonucleases were incubated first with a 1:300 dilution of biotinylated anti-hemaglutinin (Covance) for 30 min at 4° C. in a buffer containing 180 mM KCl, 10 mM NaCl, 10 mM HEPES, 0.1% galactose, 0.2% BSA, and pH 8.25. Yeast were then washed, and incubated with pre-conjugated streptavidin-phycoerythrin(PE):biotin-DNA-Alexa fluor 647, in the same buffer as above with 580 mM KCl, for 30 min at 4° C. Cells were again washed, and transferred to a buffer containing 150 mM KCl, 10 mM NaCl, 10 mM HEPES, 5 mM K-Glu, 0.05% BSA, and pH 8.25, with 7 mM CaCl₂ or MgCl₂ for control and cleavage reactions, respectively. The yeast were incubated for 5 to 30 min at 37° C. to allow catalysis; the reaction was halted by centrifugation and washed with the buffer above containing 580 mM KCl. Fluorescein isothiocyanate (FITC)-conjugated anti-Myc (ICL labs) was added to the washed cells at 1:100 dilution, and allowed to incubate for at least 10 minutes prior to flow-cytometric acquisition. See FIG. 19.

Using a BD FACSAria™ II cell sorter, cells were hierarchically gated for single yeast cells surface expressing full-length enzyme. Yeast cells within these gates showing decreased Alexa-flour 647 signal (indicating catalytic activity) were sorted using maximal phase and purity masking. Sorted yeast were expanded in culture, and analyzed for increased catalytic activity. Plasmid was isolated from yeast populations and electroporated into E. coli (as above) for sequencing. All data was analyzed using FloJo® software (Tree Star).

While the preferred embodiment of the invention has been illustrated and described, it will be appreciated that various changes can be made therein without departing from the spirit and scope of the invention.

1. A method for selecting a variant or engineered I-OnuI endonuclease with a target site modification from the wild-type and directed to a site within a gene of interest comprising:
i) determining the target site for a I-OnuI endonuclease;
ii) searching a nucleic acid database for a gene of interest comprising a nucleotide sequence that is at least 40% identical to the nucleotide sequence of target site of the I-OnuI endonuclease;
iii) selecting a gene of interest comprising the nucleotide sequence that is at least 40% identical to the nucleotide sequence of the target site of a I-OnuI endonuclease and the I-OnuI endonuclease;
iv) constructing a molecular model of the I-OnuI endonuclease bound to the nucleotide sequence that is at least 40% identical to the nucleotide sequence of the target site of the I-OnuI endonuclease from the gene of interest;
v) mutating the I-OnuI endonuclease at amino acid residues that have been determined to be direct contact residues, backbone contact residues, or water-mediated contact residues with the target site of the gene of interest to form a library of variant or engineered I-OnuI endonuclease;
vi) expressing the library of variant or engineered I-OnuI endonuclease;
vii) screening the library of variant or engineered I-OnuI endonuclease for binding activity to the target sequence in the selected gene and the cleavage activity for the target sequence in the selected gene; and
viii) selecting the variant or engineered I-OnuI endonuclease that can act upon a nucleotide sequence containing a modification in the target site from the wild-type and directed to a target site within the gene of interest, wherein the binding and cleavage activity is highest for the target sequence in the gene of interest.

2. A method for selecting an engineered I-OnuI endonuclease homologue with a target site modification from the wild-type and directed to a site within a gene of interest comprising:
i) determining the target site for a I-OnuI endonuclease homologue;
ii) searching a nucleic acid database for a gene of interest comprising a nucleotide sequence that is at least 40% identical to the nucleotide sequence of target site of the I-OnuI endonuclease homologue;
iii) selecting a gene of interest comprising the nucleotide sequence that is at least 40% identical to the nucleotide sequence of the target site of the I-OnuI endonuclease homologue;
iv) constructing a molecular model of the I-OnuI endonuclease homologue bound to the nucleotide sequence that is at least 40% identical to the nucleotide sequence of the target site of the I-OnuI endonuclease homologue from the gene of interest;
v) mutating the I-OnuI endonuclease homologue at amino acid residues that have been determined to be direct contact residues, backbone contact residues, or water-mediated contact residues with the target site of the gene of interest to form a library of engineered I-OnuI endonuclease homologues;
vi) expressing the library of engineered I-OnuI endonuclease homologues;
vii) screening the library of engineered I-OnuI endonuclease homologues for binding activity to the target sequence in the selected gene and the cleavage activity for the target sequence in the selected gene; and
viii) selecting the variant or engineered I-OnuI endonuclease homologue that can act upon a nucleotide sequence containing a modification in the target site from the wild-type and directed to a target site within the gene of interest, wherein the binding and cleavage activity is highest for the target sequence in the gene of interest.

3. A method for selecting an engineered hybrid of two I-OnuI or I-OnuI homologue domains with a target site modification from the wild-type and directed to a site within a gene of interest comprising:
i) determining the target site for a hybrid of two I-OnuI or I-OnuI homologue domains;
ii) searching a nucleic acid database for a gene of interest comprising a nucleotide sequence that is at least 40% identical to the nucleotide sequence of target site of the hybrid of two I-OnuI or I-OnuI homologue domains;
iii) selecting a gene of interest comprising the nucleotide sequence that is at least 40% identical to the nucleotide sequence of the target site of the hybrid of I-OnuI or I-OnuI homologue domains;
iv) constructing a molecular model of the hybrid of two I-OnuI or I-OnuI homologue domains bound to the nucleotide sequence of the nucleotide sequence that is at least 40% identical to the nucleotide sequence of the target site of the hybrid of two I-OnuI or I-OnuI homologue domains from the gene of interest;
v) mutating the hybrid of two I-OnuI or I-OnuI homologue domains at amino acid residues that have been determined to be direct contact residues, backbone contact residues, or water-mediated contact residues with the target site of the gene of interest to form a library of engineered hybrids of two I-OnuI or I-OnuI homologue domains;
vi) expressing the library of engineered hybrids of I-OnuI or I-OnuI homologue domains;
vii) screening the library of engineered hybrids of I-OnuI or I-OnuI homologue domains for binding activity to the target sequence in the selected gene and the cleavage activity for the target sequence in the selected gene; and
viii) selecting the engineered hybrid of two I-OnuI or I-OnuI homologue domains that can act upon the nucleotide sequence containing a modification in the target site from the wild-type and directed to a target site within the gene of interest, wherein the binding and cleavage activity is highest for the target sequence in the gene of interest.

4. A method for producing an engineered I-OnuI endonuclease, an engineered I-OnuI endonuclease homologue, or a hybrid of two I-OnuI or I-OnuI homologue domains with a target site modification from the wild-type and directed to a site within a gene of interest comprising:
i) determining the nucleotide sequence of the gene of interest;
ii) searching a nucleic acid database comprising the target sites for I-OnuI endonuclease, I-OnuI endonuclease homologues, and hybrids of two I-OnuI or I-OnuI homologue domains for a I-OnuI endonuclease, I-OnuI endonuclease homologues, and hybrids of two I-OnuI or I-OnuI homologue domains comprising a nucleotide sequence that is at least 40% identical to a nucleotide sequence of within the gene of interest;
iii) selecting the I-OnuI endonuclease, I-OnuI endonuclease homologues, and hybrids of two I-OnuI or I-OnuI homologue domains comprising the I-OnuI endonuclease, I-OnuI endonuclease homologues, and hybrids of two I-OnuI or I-OnuI homologue domains with the target site nucleotide sequence that is at least 40% identical to the nucleotide sequence within the gene of interest;
iv) constructing a molecular model of the selected I-OnuI endonuclease, I-OnuI endonuclease homologues, and hybrids of two I-OnuI or I-OnuI homologue domains bound to the nucleotide sequence of the target site that is at least 40% identical to the nucleotide sequence within the gene of interest with the nucleic acid sequence within the gene of interest;
v) mutating the selected I-OnuI endonuclease, I-OnuI endonuclease homologues, and hybrids of two I-OnuI or I-OnuI homologue domains at amino acid residues that have been determined to be direct contact residues, backbone contact residues, or water-mediated contact residues with the target site of the gene of interest to form a library of engineered I-OnuI endonuclease, I-OnuI endonuclease homologues, and hybrids of two I-OnuI or I-OnuI homologue domains;
vi) expressing the library of engineered I-OnuI endonuclease, I-OnuI endonuclease homologues, and hybrids of two I-OnuI or I-OnuI homologue domains;
vii) screening the library of engineered I-OnuI endonuclease, I-OnuI endonuclease homologues, and hybrids of two I-OnuI or I-OnuI homologue domains for binding activity to the target sequence in the gene of interest and the cleavage activity for the target sequence in the gene of interest; and
viii) selecting the engineered I-OnuI endonuclease, I-OnuI endonuclease homologues, and hybrids of two I-OnuI or I-OnuI homologue domains that can act upon a nucleotide sequence containing a modification in the target site from the wild-type and directed to a target site within the gene of interest, wherein the binding and cleavage activity is highest for the target sequence in the gene of interest.

5. The method of claim 1, wherein the I-OnuI homologue comprises about 25% or greater amino acid sequence identity extending over at least 200 amino acids and including both LAGLIDADG sequence motifs with the amino acid sequence of I-OnuI (SEQ ID NO: 35).

6. The method of claim 5, wherein the I-OnuI homologue comprises an amino acid sequence that is highly conserved when compared to the LAGLIDADG motifs (amino acid residues 12 to 24 of SEQ ID NO: 35 and amino acid residues 170 to 181 of the I-OnuI amino acid sequence in SEQ ID NO: 35.

7. The method of claim 6, wherein the I-OnuI homologue further comprises high amino acid conservation within a “Loop” sequence adjacent to the first LAGLIDADG helix corresponding to amino acid residues 97 to 103 of SEQ ID NO: 35.

8. The method of claim 7, wherein the I-OnuI homologue demonstrates an overall spacing between the end and beginning of the two LAGLIDADG motifs of between about 162 and 182 amino acid residues.

9. The method of claim 5, wherein the homologues of I-OnuI is I-AabI (SEQ ID NO: 52), I-AaeI (SEQ ID NO: 53), I-ApaI (SEQ ID NO:54), I-CkaI (SEQ ID NO:55), I-CpaI (SEQ ID NO: 56), I-CapIII (SEQ ID NO:57), I-CapIV (SEQ ID NO:58), I-CpaV (SEQ ID NO:59), I-CraI (SEQ ID NO:60), I-EjeI (SEQ ID NO:61), I-GpeI (SEQ ID NO:61), (SEQ ID NO:63), I-GzeI (SEQ ID NO:64), I-GzeII (SEQ ID NO:65), I-GzeIII (SEQ ID NO:66), I-HjeII (SEQ ID NO: 67), I-LtrI (SEQ ID NO:68), I-LtrII, (SEQ ID NO:69) I-MpeI (SEQ ID NO:70), I-MveI (SEQ ID NO:71), I-NcrI (SEQ ID NO:72), I-NcrII (SEQ ID NO:73), I-OheI (SEQ ID NO:74), I-OsoI (SEQ ID NO:75), I-OsoII (SEQ ID NO:76), I-OsoIII (SEQ ID NO:77), I-OsiIV (SEQ ID NO:78), I-PanI (SEQ ID NO:79), I-PanII (SEQ ID NO:80), I-PanIII (SEQ ID NO:81), I-PnoI (SEQ ID NO:82), I-ScuI (SEQ ID NO:83), I-SmaI (SEQ ID NO:84), or I-SscI (SEQ ID NO:85).

10. The method of claim 2, wherein the I-OnuI homologue comprises about 25% or greater amino acid sequence identity extending over at least 200 amino acids and including both LAGLIDADG sequence motifs with the amino acid sequence of I-OnuI (SEQ ID NO: 35).

11. The method of claim 10, wherein the I-OnuI homologue comprises an amino acid sequence that is highly conserved when compared to the LAGLIDADG motifs (amino acid residues 12 to 24 of SEQ ID NO: 35 and amino acid residues 170 to 181 of the I-OnuI amino acid sequence in SEQ ID NO: 35.

12. The method of claim 11, wherein the I-OnuI homologue further comprises high amino acid conservation within a “Loop” sequence adjacent to the first LAGLIDADG helix corresponding to amino acid residues 97 to 103 of SEQ ID NO: 35.

13. The method of claim 12, wherein the I-OnuI homologue demonstrates an overall spacing between the end and beginning of the two LAGLIDADG motifs of between about 162 and 182 amino acid residues.

14. The method of claim 3, wherein the I-OnuI homologue comprises about 25% or greater amino acid sequence identity extending over at least 200 amino acids and including both LAGLIDADG sequence motifs with the amino acid sequence of I-OnuI (SEQ ID NO: 35).

15. The method of claim 14, wherein the I-OnuI homologue comprises an amino acid sequence that is highly conserved when compared to the LAGLIDADG motifs (amino acid residues 12 to 24 of SEQ ID NO: 35 and amino acid residues 170 to 181 of the I-OnuI amino acid sequence in SEQ ID NO: 35.

16. The method of claim 15, wherein the I-OnuI homologue further comprises high amino acid conservation within a “Loop” sequence adjacent to the first LAGLIDADG helix corresponding to amino acid residues 97 to 103 of SEQ ID NO: 35.

17. The method of claim 16, wherein the I-OnuI homologue demonstrates an overall spacing between the end and beginning of the two LAGLIDADG motifs of between about 162 and 182 amino acid residues.

18. The method of claim 4, wherein the I-OnuI homologue comprises about 25% or greater amino acid sequence identity extending over at least 200 amino acids and including both LAGLIDADG sequence motifs with the amino acid sequence of I-OnuI (SEQ ID NO: 35).

19. The method of claim 18, wherein the I-OnuI homologue comprises an amino acid sequence that is highly conserved when compared to the LAGLIDADG motifs (amino acid residues 12 to 24 of SEQ ID NO: 35 and amino acid residues 170 to 181 of the I-OnuI amino acid sequence in SEQ ID NO: 35.

20. The method of claim 19, wherein the I-OnuI homologue further comprises high amino acid conservation within a “Loop” sequence adjacent to the first LAGLIDADG helix corresponding to amino acid residues 97 to 103 of SEQ ID NO: 35.


SEQUENCE LISTING (122 sequences)

SEQ ID NO: 1 - DNA, 20 bp, Artificial Sequence
  I-OnuI target
ttccacttattcaaccttta

SEQ ID NO: 2 - DNA, 22 bp, Artificial Sequence
  Complementary sequence
taaaaggttgaataagtggaaa

SEQ ID NO: 3 - DNA, 22 bp, Artificial Sequence
  designed and/or selected mutations in I-OnuI enzyme scaffold
tatccacttattcaacctttta

SEQ ID NO: 4 - DNA, 22 bp, Artificial Sequence
  designed and/or selected mutations in I-OnuI enzyme scaffold
tctccacttattcaacctttta

SEQ ID NO: 5 - DNA, 22 bp, Artificial Sequence
  designed and/or selected mutations in I-OnuI enzyme scaffold
tgtccacttattcaacctttta

SEQ ID NO: 6 - DNA, 22 bp, Artificial Sequence
  designed and/or selected mutations in I-OnuI enzyme scaffold
ttttcacttattcaacctttta

SEQ ID NO: 7 - DNA, 22 bp, Artificial Sequence
  designed and/or selected mutations in I-OnuI enzyme scaffold
tttgcacttattcaacctttta

SEQ ID NO: 8 - DNA, 22 bp, Artificial Sequence
  designed and/or selected mutations in I-OnuI enzyme scaffold
tttacacttattcaacctttta

SEQ ID NO: 9 - DNA, 22 bp, Artificial Sequence
  designed and/or selected mutations in I-OnuI enzyme scaffold
tttcgacttattcaacctttta

SEQ ID NO: 10 - DNA, 22 bp, Artificial Sequence
  designed and/or selected mutations in I-OnuI enzyme scaffold
tttcaacttattcaacctttta

SEQ ID NO: 11 - DNA, 22 bp, Artificial Sequence
  designed and/or selected mutations in I-OnuI enzyme scaffold
tttccatttattcaacctttta

SEQ ID NO: 12 - DNA, 22 bp, Artificial Sequence
  designed and/or selected mutations in I-OnuI enzyme scaffold
tttccagttattcaacctttta

SEQ ID NO: 13 - DNA, 22 bp, Artificial Sequence
  designed and/or selected mutations in I-OnuI enzyme scaffold
tttccaattattcaacctttta

SEQ ID NO: 14 - DNA, 22 bp, Artificial Sequence
  designed and/or selected mutations in I-OnuI enzyme scaffold
tttccacgtattcaacctttta

SEQ ID NO: 15 - DNA, 22 bp, Artificial Sequence
  designed and/or selected mutations in I-OnuI enzyme scaffold
tttccacctattcaacctttta

SEQ ID NO: 16 - DNA, 22 bp, Artificial Sequence
  designed and/or selected mutations in I-OnuI enzyme scaffold
tttccacatattcaacctttta

SEQ ID NO: 17 - DNA, 22 bp, Artificial Sequence
  designed and/or selected mutations in I-OnuI enzyme scaffold
tttccactgattcaacctttta

SEQ ID NO: 18 - DNA, 22 bp, Artificial Sequence
  designed and/or selected mutations in I-OnuI enzyme scaffold
tttccactcattcaacctttta

SEQ ID NO: 19 - DNA, 22 bp, Artificial Sequence
  designed and/or selected mutations in I-OnuI enzyme scaffold
tttccactaattcaacctttta

SEQ ID NO: 20 - DNA, 22 bp, Artificial Sequence
  designed and/or selected mutations in I-OnuI enzyme scaffold
tttccactttttcaacctttta

SEQ ID NO: 21 - DNA, 22 bp, Artificial Sequence
  designed and/or selected mutations in I-OnuI enzyme scaffold
tttccacttattcagcctttta

SEQ ID NO: 22 - DNA, 22 bp, Artificial Sequence
  designed and/or selected mutations in I-OnuI enzyme scaffold
tttccacttattcatcctttta

SEQ ID NO: 23 - DNA, 22 bp, Artificial Sequence
  designed and/or selected mutations in I-OnuI enzyme scaffold
tttccacttattcaagctttta

SEQ ID NO: 24 - DNA, 22 bp, Artificial Sequence
  designed and/or selected mutations in I-OnuI enzyme scaffold
tttccacttattcaatctttta

SEQ ID NO: 25 - DNA, 22 bp, Artificial Sequence
  designed and/or selected mutations in I-OnuI enzyme scaffold
tttccacttattcaaactttta

SEQ ID NO: 26 - DNA, 22 bp, Artificial Sequence
  designed and/or selected mutations in I-OnuI enzyme scaffold
tttccacttattcaacatttta

SEQ ID NO: 27 - DNA, 22 bp, Artificial Sequence
  designed and/or selected mutations in I-OnuI enzyme scaffold
tttccacttattcaacgtttta

SEQ ID NO: 28 - DNA, 22 bp, Artificial Sequence
  designed and/or selected mutations in I-OnuI enzyme scaffold
tttccacttattcaacttttta

SEQ ID NO: 29 - DNA, 22 bp, Artificial Sequence
  designed and/or selected mutations in I-OnuI enzyme scaffold
tttccacttattcaaccattta

SEQ ID NO: 30 - DNA, 22 bp, Artificial Sequence
  designed and/or selected mutations in I-OnuI enzyme scaffold
tttccacttattcaacccttta

SEQ ID NO: 31 - DNA, 22 bp, Artificial Sequence
  designed and/or selected mutations in I-OnuI enzyme scaffold
tttccacttattcaacctatta

SEQ ID NO: 32 - DNA, 22 bp, Artificial Sequence
  designed and/or selected mutations in I-OnuI enzyme scaffold
tttccacttattcaacctctta

SEQ ID NO: 33 - DNA, 22 bp, Artificial Sequence
  designed and/or selected mutations in I-OnuI enzyme scaffold
tttccacttattcaacctgtta

SEQ ID NO: 34 - DNA, 20 bp, Artificial Sequence
  MAO-B
gtccacatatttaacctttt

SEQ ID NO: 35 - protein, 306 aa, Artificial Sequence
  Ophiostoma novo-ulmi
INISAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 36 - protein, 303 aa, Artificial Sequence
  T48C/K80R
SAYMSRRESINPWILTGFADAEGSFLLRIRNNNKSSVGYSTELGFQICLHNKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 37 - protein, 303 aa, Artificial Sequence
  T48C/S72A/K80R
SAYMSRRESINPWILTGFADAEGSFLLRIRNNNKSSVGYSTELGFQICLHNKDKSILENIQSTWKVGVIANAGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 38 - protein, 303 aa, Artificial Sequence
  T48C/S72C/K80R
SAYMSRRESINPWILTGFADAEGSFLLRIRNNNKSSVGYSTELGFQICLHNKDKSILENIQSTWKVGVIANCGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 39 - protein, 303 aa, Artificial Sequence
  T48C/S72N/K80R
SAYMSRRESINPWILTGFADAEGSFLLRIRNNNKSSVGYSTELGFQICLHNKDKSILENIQSTWKVGVIANNGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 40 - protein, 303 aa, Artificial Sequence
  T48C/S72V/K80R
SAYMSRRESINPWILTGFADAEGSFLLRIRNNNKSSVGYSTELGFQICLHNKDKSILENIQSTWKVGVIANVGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 41 - protein, 303 aa, Artificial Sequence
  T48M/S72R/K80R
SAYMSRRESINPWILTGFADAEGSFLLRIRNNNKSSVGYSTELGFQICLHNKDKSILENIQSTWKVGVIANRGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 42 - protein, 303 aa, Artificial Sequence
  N32L/S40R/T48M/S72R/K80R
SAYMSRRESINPWILTGFADAEGSFLLRIRNLNKSSVGYRTELGFQIMLHNKDKSILENIQSTWKVGVIANRGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 43 - protein, 303 aa, Artificial Sequence
  N32L/S40R/T48M/S72A/K80R
SAYMSRRESINPWILTGFADAEGSFLLRIRNLNKSSVGYRTELGFQIMLHNKDKSILENIQSTWKVGVIANAGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 44 - protein, 303 aa, Artificial Sequence
  N32R/S40R/T48M/S72R/K80R
SAYMSRRESINPWILTGFADAEGSFLLRIRNRNKSSVGYRTELGFQIMLHNKDKSILENIQSTWKVGVIANRGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 45 - protein, 303 aa, Artificial Sequence
  N32I/S40R/T48M/S72R/K80R
SAYMSRRESINPWILTGFADAEGSFLLRIRNINKSSVGYRTELGFQIMLHNKDKSILENIQSTWKVGVIANRGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 46 - protein, 303 aa, Artificial Sequence; identical to SEQ ID NO: 42
  N32L/S40R/T48M/S72R/K80R

SEQ ID NO: 47 - protein, 303 aa, Artificial Sequence
  N32L/S40R/T48M/S72R/K80R/R133C
SAYMSRRESINPWILTGFADAEGSFLLRIRNLNKSSVGYRTELGFQIMLHNKDKSILENIQSTWKVGVIANRGDNAVSLRVTRFEDLKVIIDHFEKYPLITQKLGDYMLFKQAFCVMENKEHLKINGIKELVCIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 48 - protein, 303 aa, Artificial Sequence
  N32L/S40R/T48M/S72R/K80R/K229R (R3#3)
SAYMSRRESINPWILTGFADAEGSFLLRIRNLNKSSVGYRTELGFQIMLHNKDKSILENIQSTWKVGVIANRGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVF

SEQ ID NO: 49 - protein, 303 aa, Artificial Sequence
  N32L/S40R/T48M/S72R/K80R/E178D/K229R
SAYMSRRESINPWILTGFADAEGSFLLRIRNLNKSSVGYRTELGFQIMLHNKDKSILENIQSTWKVGVIANRGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGDGCFFVNLIKSKSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVF

SEQ ID NO: 50 - protein, 303 aa, Artificial Sequence
  N32L/S40R/TriM/S72R/K80R/K229H (R3 #6)
SAYMSRRESINPWILTGFADAEGSFLLRIRNLNKSSVGYRTELGFQIMLHNKDKSILENIQSTWKVGVIANRGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNHSEFSWLDFVVTKFSDINDKIIPVFQENTLIGVKLEDFEDWCKVAKLIEEKKHLTESGLDEIKKIKLNMNKGRVF

SEQ ID NO: 51 - protein, 303 aa, Artificial Sequence
  N32L/S40R/T48M/S72R/K80R/K229Y (R3 #8)
SAYMSRRESINPWILTGFADAEGSFLLRIRNLNKSSVGYRTELGFQIMLHNKDKSILENIQSTWKVGVIANRGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNYSEFSWLDFVVTKFSDINDKIIPVFQENTLIGVKLEDFEDWCKVAKLIEEKKHLTESGLDEIKKIKLNMNKGRVF

SEQ ID NO: 52 - protein, 329 aa, Ascocalyx abietina
STSDNNGNIKINPWFLTGFIDGEGCFRISVTKINRAIDWRVQLFFQINLHEKDRALLESIKDYLGVGKIHISGKNLVQYRIQTFDELTILIKHLKEYPLVSKKRADFELFNTAHKLIKLNEHLNKEGILKLVSLKASLNLGLSEVLKLAFPNVISATRLTDFTVNIPDPHWLSGFASAEGCFMVGIAKSSASSTGYQVYLTFILTQHVRDEFLMKCLVDYFNWGRLARKRNVYEYQVSKFSDVEKLLVFFDKYPILGEKAKDLFDFCSVSDLMKSKTHLTEIGVAKIRKIKEGMNRGRFYCRQLGKILSLKPKINLLDQYFSLKIRIAM

SEQ ID NO: 53 - protein, 306 aa, Ascocalyx abietina
PIQIFPINPWFITGFTDAEGSFMVSITKNENSKLKWGIYPSFAIHIHNKDISLLNQIQKTLGVGNVRKNSKTTALFRVDNLKELQVIINHFDKYPLVSFKASDYIIFKKCYNLIMQKQHLTQKGFEEILSLKYKLNKGLPDNLKKTFPHIIPIERPEYIFINIPNPYWISGFASGDSTFSVSIENSNNKLGKRVRLIFGTCLHIRDKELLIGMANYFYNSFENLNKSVLNKQNKLSDSIEHKYKYNYDSPTTSLLQIKKYSDIRDIIIPFFNKYPILGVKHLDFYDFKLISNLIDNKEHLTQDRFK

SEQ ID NO: 54 - protein, 305 aa, Amoebidium parasiticum
STSIPLQTLTPFFVTGITDAEGCFSVGISKHKQLRTGWCVKPVFSITLHERDEALLKQLLSFFGKGGIFRHGPTTLQVRFESKNALWAVVKHFDKYPLISQKQADYLLWKKAVELFSDKEHLTLAGLFEVVALRASINWGLPPRLRKAFPLIASKIRPEVKKPVVPNPEWMAGFISGEGCFIVEILENKEMRLGYSVKIKFNIPQHKRDIILLQVLNEYLGGGGNFHQAKNRDVMEFRFQDFSIIDEQIVPFIRSFPIQGMKIHDFNDWCQAIDIVRRKGHLTPEGLAEIQKLEEGMNTGRKEKK

SEQ ID NO: 55 - protein, 306 aa, Cordyceps kanzashiana
KAVLDNEQISPNFTLNPWFISGFIDGEGCFRISFTKKDNLIGWRVQLFFQITLHQKDKLLLESILNYWGVGKVYKSGKDTLQYKIQSFTEIKAILNHLDKYPLISKKRADYELFKNAYYIILNKEHLTKLGLRKLVALRASLNLGLPKQLKTAFPDIIPMTRPLVEDIKIRNIHWLIGFVSAEGCFLVGLRKSSSYSAGYQVYLTFIITQHIRDEQLMNSLIEYLGCGNINKKKEVFEYQVSKYSDLTEKIIPLFNKYPIIGQKYEDFKDFEKVAILMENRKHLTIAGVDEIRSIKVNMNKGRKLN

SEQ ID NO: 56 - protein, 300 aa, Cryphonectria kanzashiana
DLSTSLNPWVVTGLVDAEGSFNITVVKNNNSKLGWVTKLRLEMSMLKKDRFTLEQLINYFGGGGIRVLDDNNLRFYIESLKDLTTVINHFDKYPLFTKKQEDYLLFKQVFELFKNKNHLTMEGLRQIVAIKALLRNKGLSNSLLEAFPGITPATLPITDTITFGVDKSILSPWLAGFTSGDGNFYISIGEGPKHVQVQLVFTLTQHIRDQALMNSLISYLGCGNIKHNEKNSWLQFIVTKFSDIDEKIIPIFKENKILGEKFKDFQDWCRAAELMKNKAHLTPSGLEEIRKLKAGMNRGR

SEQ ID NO: 57 - protein, 302 aa, Cryphonectria parasitica
KPTNTSSSFNPWFLTGFSDAECSFSILIQANSKYSTGWRIKPVFAIGLHKKDLELLKRIQSYLGVGKIHIHGKDSIQFRIDSPKELEVIINHFENYPLVTAKWADYTLFKKALDVILLKEHLSQKGLLKLVGIKASLNLGLNGSLKEAFPNWEELQIDRPSYVFKGIPDPNWISGFASGDSSFNVKISNSPTSLLNKRVQLRFGIGLNIREKALIQYLVAYFDLSDNLKNIYFDLNSARFEVVKFSDITDKIIPFFDKYSIQGKKSLDYINFKEVADIIKSKNHLTSEGFQEILDIKASMNK

SEQ ID NO: 58 - protein, 285 aa, Cryphonectria parasitica
MSNFTLGYLGNIFIKTNNMKNLDNNWIRWFTGFCDAEGNFQVYPKKRVLKSGEVSKYNVGLGFHLSLHSRDAELLHVIHDKLNNVGVYYEFKNRNEARIAVNDRAGLKVIVDVFGIFPLVTIHQYTRYCLLKEYLINDIKEFKTLEKFNAFKDKCLDNIDLSSKVLSGLESQINSGLLDSWIVGLINGEGCFYLNKGRCNFFIEHTDLQALEIIKKRLSFSPKIVARSPRARDVGKDIKPTYMLIVSSKKDIESLIELLDSKRVVPLAGHKLMQYNEWKHTWFNK

SEQ ID NO: 59 - protein, 316 aa, Cryphonectria parasitica
TNLTEFYQWFVGFSDGESSFQIQIKYSDQDKTKIRGVNFSFTISLHIDDIAVLKFIKDKLEIGNITVKKTRAACVFSVTNQEGLNKLISIFDKYNLNTTKYLDYLDYLDFRKAFLLYQDKDINFNKENLNILINQIVDLKSRMNSERTFFDMSVCNTSFSTNSLHSGINKNWLLGFIEAEGSFFISITDIEPSFSIELSNAQMFLLEKIKDFLISDLGFDGYSLFQLKSSSFSVISVNKQKVKPSAILLIKNIRVLNNYLVPYLSTEVFKSKKGQDFEDWKLICEAVYKGSHKIDEIRELILRLTYSMNNYRLSTN

SEQ ID NO: 60 - protein, 303 aa, Cryphonectria kanzashiana
SQNNINNCLSWYVSGLVDAEGSFGVTLVKKESSVTGYSVLIYFEIALNKKDKQLLETIKQVLGIDKNLYYNENDNTLKLRISNLDVLIKNVIPHFNKYPLFTQKRVDFLLMCKIIELVEEKKHLTKEGLNEILNIKAAMNLGLSDKLKNEFLTVKSLTRLSIDTGIPNKDWLEGFMEGESCFFVRVYNSPKSKLKLAVQLAFIITQHSRDKILLENISKLLKCGRVETRKSGDACDFVVTSFKDFTKYMIPYWKDYPLIGNKSKDCLDFIRVYEIMLNKGHLTEEGLTKIEVEIKNSMNTKRE

SEQ ID NO: 61 - protein, 299 aa, Elaphocordyceps jezoensis
QLQSSLNPWFFTGFADAEGSFSILIQTNSKYTTGWRIKPIFTIGLHKKDLDLLLNIQAYLGIGKIHIHGIDSIQFRVDSLKELQVLINHFDNFPLVTAKLADYILFKKAFAIILLKEHLSSEGLSKIVGIKASLNLGLNQSIKEAFPNWAELQINRPDYTFKCIPDPNWMAGFASGDSSFNVKISKSPTSLLGQRVQLRFAIELNSREKNLIQHLSAYFKLTYILKNVYIDNQVARFQIVNSSAIFYKIIPFFEKYLIQGKKSLDFISFKEVALIVQNKDHLKSEGFKKILDIKAKMNE

SEQ ID NO: 62 - protein, 308 aa, Grosmannia penicillata
PTRNESINPWVLTGFADAEGSFILRIRNNNKSSAGYSTELGFQITLHKKDISILENIQSTWKVGVIANSGDNAVSLKVTRFEDLRVVLNHFEKYPLITQKLGDYLLFKQAFSVMENKEHLKIEGIKRLVGIKANLNWGLTDELKEAFVASGGENIFVASGGERSLINKNIPNSGWLAGFTSGEGCFFVSLIKSKSKLGVQVQLVFSITQHARDRALMDNLVTYLGCGYIKEKKKSEFSWLEFVVTKFSDIKDKIIPVFQVNNIIGVKLEDFEDWCKVAKLIEEKKHLTESGLEEIRNIKLNMNKGRVL

SEQ ID NO: 63 - protein, 303 aa, Grosmannia piceiperda
ATVTPLIDPWFITGFADAESSFVVSIKRNKKIKCGWNVVTRFQIALSQKDLALLERIKSYFKDAGNIYIKSDKVSVDWHVTSVKDLKIILDHFDKYPLKTEKLADYILFKEVFNIILTKQHLTVEGIQKIVAIRASINKGLYGELKAAFPNIIPVQRPKIDDRFIIDIQPWWVAGFTEGEGCFSVVVTNSPSTKSGFSASLIFQITQHSRDIVLMQNIIKFLGCGRIHKRSKEEAVDILVTKFSDLTEKVIPFFESIPLQGLKLKNFTDFSKAADIIKVKGHLTPKGLDKILQIKLGMNTRRI

SEQ ID NO: 64 - protein, 301 aa, Gibberella zeae
SLEQSSLPPKLDPSYVTGFTDGEGSFILTIIKDNKYKLGWRVACRFVISLHKKDLVLLNSLKNFFNTGSVFLMGKGAAQYRVESLTGLSIIINHFDRYPLNTKKQADYMLFKLAYNLIINKSHLTEKGLSELVSLKAVMNNGLKDELKIAYPNITPVLRPEIPLSLNIDPLWLAGFTDAEGCFSVVVFKSKTSKIGEAVKLSFIITQSVRDEFLIKSLIEYLGCGYTSLDGRGAIDFKVSDFSSLKNIIIPFYDKYYIHGNKSLDFKDFSRVVTLMENKKHLTKQGLDEIKKIRNAMNTNR

SEQ ID NO: 65 - protein, 298 aa, Gibberella zeae
EVVPINPWFVTGFTDAEGSFMIHLEKNKDKWRVRPTFQIKLDIRDLSLLEEIKIYFNNTGSINTSNKECVYKVRSLKDISIIISHFDKYNLITQKKADFELFKLIINKLNSQEHLSYEVGATVLQEIISIRASMNLGLSSSVKEDFPHIIPVIRPLIENMVIPHPEWMAGFVSGEGSFSVYTTSDDKYVSLSFRVSQHNKDKQLLKSFVDFFGCGGFNYHNKGNKAVIFVTRKFEDINDKIIPLFNEYKIKGVKYKDFKDWSLVAKMIESKSHLTTNGYKEICKIKENMNSYRKSSVN

SEQ ID NO: 66 - protein, 298 aa, Gibberella zeae
KESFKLNPWFITGFTDGDGSFTISTSKKKSGTGWKIHPTFTIGLDIKDLDILIQFKAYFNAGKIYKSKRGIIYYTIGSTKDLLKHVLPHFDKYPLSSLKLKDYLVFKRILLLMQKGEHQSLSGLFKIFSERANLNKGLPKVVEEEYPDLKPAVIPELKIAPTINPDWLAGFITAEASFFISIYPSKDRKVGYAVSLVFSLSQHAKDLDLLKRIADSLECGIVRKHKSREAVELVITKSEDINQKLTPLLSKHTLSGVKLLDFERFKKASILINSKAHLTSEGIKLIKDIKDTMYNRGL

SEQ ID NO: 67 - protein, 301 aa, Hypocrea jecorina
NLIIDPNFLTGFTDAEGSFVLSITKSDNVKSGWVIKPRFSISLHKKDKFVLEAIKNYLGVGEIYTQGTDSIQYRVFSIKDLQLVIDHFDKYPLISQKFGDYSLFKQAYLLLINKEHLTPEGLLKIIAIRASINNGLSEALKEAFPDVIPVMKPVPRGSRNNISIYNPQWLAGFTSGEGSFGVKVRKAKENSNAFIELIFQINQHVRDKQLMACIAEYLECGKIYKHSLNAIVYRVSKTSNLTEKVIPFFIKYPILGIKALDFKDFCSLAELISNKAHYTEEGLNKIISIKANMNTGRIWEN

SEQ ID NO: 68 - protein, 308 aa, Leptoyraphium truncatum
FPVQARNDNISPWTITGFADAESSFMLTVSKDSKRNTGWSVRPRFRIGLHNKDVTILKSIREYLGAGIITSDKDARIRFESLKELEVVINHFDKYPLITQKRADYLLFKKAFYLIKNKEHLTEEGLNQILTLKASLNLGLSEELKEAFPNTIPAEKLLVTGQEIPDSNWVAGFTAGEGSFYIRIAKNSTLKTGYQVQSVFQITQDTRDIELMKNLISYLNCGNIRIRKYKGSEGIHDTCVDLVVTNLNDIKEKIIPFFNKNHIIGVKLQDYRDWCKVVTLIDNKEHLTSEGLEKIQKIKEGMNRGRSL

SEQ ID NO: 69 - protein, 303 aa, Leptoyraphium truncatum
MINLKNNIEYLNWYICGLVDAEGSFGVNVVKHATNKTGYAVLTYFELAMNSKDKQLLELIKKTFDLECNIYHNPSDDTLKFKVSNIEQIVNKIIPFFEKYTLFSQKRGDFILFCKVVELIKNKEHLTLNGLMKIISIKAAMNLGLSENLKKEFPGCLSVKRPEFGLSNLNKRWLAGFIEGEACFFVSIYNSPKSKLGKAVQLVFKITQRIRDKILIESIVELLNCGRVEVRKSNEACDFTVTSIKEIENYIIPLFNEYPLIGQKLKNYEDFKLIFDMMKTKDHLTEEGLSKIIEIKNKMNTNR

SEQ ID NO: 70 - protein, 310 aa, Moniliophthora perniciosa
NSPASINQFKNKLDPWWITGFTDAEGSFGLYIYKNSKFKTGWHAYLVFSISLHEKDRDILSQIQNFFGFGGIHSHGSNSLKYTVKSLNELQIIIDHFDKYLLITSKLNDYKLFKLAYNLFINKENLSIEGIEKLVAIKSSMNLGLKSQLKLAFPQISKHKDIYRDCFAYSNIKKIPDPYWVSGFTSGEGNFMIDIIKSKSSKLGITAGLRFTITQHFRDKQLMISLIDFFNCGHLNIRNNNCFNLTIRKFTDLDTKIIPFFSKYPIIGDKLLNFQNFIEASKLINNKDHLTIEGLKKINEIKNGMNTKRI

SEQ ID NO: 71 - protein, 295 aa, Mortierella verticillata
MNKNNSNLTWFITGLTEAEGCFNINIYKTKAGKKTAKLRFSIAVMENDLELLKLVKDCFNCGTISESRTNGMRYFTVSKISDINNIIIPHFQSYLLRGTKLLDFEDWVLAANIIITKSHLTEEGIEKLQLLFDGMNRKRNKLENFLPDHCNKNSSLFIPINGNYISGFIAGDGSINIHPFSLTFDSLKFCSIFLSITQHKNNLFLMNEIKDFFNVNNKLKIQSNNSVQLLIENKEFFRSTLIPFFNKYPLHGIKLINLNKIIKILELINKYGINRSNSYTSDIRKEIINIWFAET

SEQ ID NO: 72 - protein, 316 aa, Neurospora crassa
HTSCVWAGLNPSFITGFSDAEGSFVVTILKNPRYKIGWNVQARFQIKLNEKDRALLLLIQNYFDNIGYISKINDRSTVEFRVSDITSLNNIIIPHFEKYQLITNKYGDLVIFKQIVSLMLENKHTTLEGLKEILEHRASLNWGLSKTLKESFPSIIPVKRVKIENNILSNLSSLPLLPRGGNWVAGFSSGEANFFITMSGTKVWLRFSIAQDSRDILLLKSLVKFFNCGYIAQYKNRKVCEFIVTKINDIIIYIIPFFDQYKIEGSKYNDYVKFKEAAILIKNKEHLTEKGLNKIIELKNSLPPPASLEGGMNKNI

SEQ ID NO: 73 - protein, 310 aa, Neurospora crassa
  misc_feature (191)..(192): Xaa can be any naturally occurring amino acid
NNKNTFYLNPDYITGFVDGEGCFSLSLFKDDRRLNGWQVKPIFSISLHKKDISLLEAIQRTFKVGKIYKHGIDSIQYRVSSLKNLQIITDHFDSYPLITQKRVDYLLFKQAIALIKNKEHLSLEGLLKLVGIKATLRSSWPNLKKVFPTVKAAVRPSVIYITSDVKVKSLNWIRGFIEGEGCFQVITQNSXXPKGRNVWLRFSLTQHIKDEELLKDIAIYLNMGRYYKSPTRNEGQYLITIFSDINNKLIPFLKEYPLLGVKQEDFLDFVKIAKLIESKTHLTDEGLDTIKLIQSNMNSKRIIKEEERKV

SEQ ID NO: 74 - protein, 303 aa, Ophiocordyceps heteropoda
FKSMIFNRKVFFFFYIHKKTAEGSFSILIQTNNKYATGWRIKPIFTIGLHKKDLDLLNKIQSYLGIGKIHIHGKDSIQFRVDSLKDLQILLNHFENFPLVTAKLADYILFKKAFDIILLKEHLSQEGLLKLVGIKASLNLGLNQNIKKAFSNWEELQVNRPNYTFKSIPDSNWMAGFASGDSSFNIKTSKSTTSALGQRVQLRFAIELNIREKDFVEHLPAYFKLRDPLKNVYIDNKVARFQIVNYFDITDKILPFFEKYLIQGKKSLDFISFKEVALIIKNKDHLNLEGFKEILDIKDRMNK

SEQ ID NO: 75 - protein, 298 aa, Ophiocordyceps sobolifera
QNNINSRLSWYISGLVDAEGSFGVTLNRKESSIIGYSILIYFEIALNQKDKQLLETIKQVLGIDKSLYYNKRDNTLKLRVSNLYVLINNVIPHFNKFTQKRVDFLSMCKIIELVQWKSHLTTEGLNEILSIKATMNLGLSNKFKNEFLTVKSLTRLPVETGIPNKDWLVGFMEGESCFFVRVYNSPRSKLNRAVQLAFIITQHSRDKVLLENISRLLECGRVETRKSGDACDFVVTSFNDFNKYMIPYWKNYPLIGHKSKDCLDFIRVYEIMLTKGHLTEEGLTKIVEIKNSMNTKRK

SEQ ID NO: 76 - protein, 304 aa, Ophiocordyceps sobolifera
KDVMDCINPWFMTGFTFFFYIHKKTAEGTFSLLIIHNKNYAINWRVKAIFAIGLHTKDLDMLENIKSFWKVGNIHKHSEHSLQYRVESIKDLQVIIDHFDNYPLVTCKIIDYDIFKKAFSIIKKQEHLTEKGILKLIGLKSSLNLGLTGRLKWEFPNWKDLKINRPEYTFKGIPHPQWMAGFSSGDSSFNIKISQSLTSKLSTRVQLRFSIGLHIREKELIEYCVKYFNLIEGKYVYLKSNSVTLEITNFNDIANVIIPFFEKYPIKGKKSLDFLEFNKVVGVVKNKQHLTQEGLDKILIIKSR

SEQ ID NO: 77 - protein, 310 aa, Ophiocordyceps sobolifera
SNNQALNPWFITGFSDAESSFIISIYKDENNKLKWRVSAYFSIHVHIKDLPLLELIQKTLGVGIVRKNNKNTVLFRVSNMQELQIVIDHFKRYPLISAKYSDFLLFEQCYNLIKQKEHLTQKGMEKILALKSNLNKGLSNELKDAFAANIVPVSRLEYKFTGIPSPFWISGFQTGDSSFSVSIEKSTSKVGKRVRLIYGTCLHIRDKDLLRGMAKYFNSLPSGCIDVVNKEISIHCNEINNTSLLQIKNNSDIENKIIPFFNEYPILGVKKLDYEDFKKVAELVKNKEHLNVEGLNKIIKIAEGMNLSRK

SEQ ID NO: 78 - protein, 297 aa, Ophiocordyceps sobolifera
DLQNTILNLHPWFITGFCDAEGSFSIYLRKKSGGSTYSEARFGISLHKDLDTLKRIQVYFKGKGSIVKHGEDSIQYAITSIEQLSTLVIPHFDNYPLISKKHADYLLFRKAVLLIRNKKHLTIEGLQEIVSIKASMNKGLSYELKEVYPNTLIVPRPLVPNSIIPDPEWVAGFTSGEGCFMIKISKSPASKLGFGIQLIFQLTQNNRDEALMKCILTYFGCGTLVKDGTKIVFFVRKFSNIKDIIIPFFNDHKIVGVKLQDFLDWYKAAEIVKAKGHLTPLGLDELKKIKVGMNRNR

SEQ ID NO: 79 - protein, 320 aa, Podospora anserina
PKSVKLSKETLNPWIVVGFSDAESSFMIRIRKNSKYKTGWTVVAVFSIAVDKKDLFLLESIKTFFGGLGSIKKHGKGTFSYRIESSEQIMKFIIPFFDKYPLITEKLGDYLLFKKVVEMLNNKEHLTETGLYKIVSLKASINKGLSEELQAAFPQCIPVFRPTVYNKIIPDPNWLAGFVSGEGCFKSILKKSSSVKVGFQSILIFQVTQHARDEKLMESLISYFKCGYIEKDPRGPWLSYIVSNFTDIYTKIIPFFLQYNIIGSKNLDFNDWCKIATLMQDKHHLTTEGLNKIISIKGGMNKGRLSSVSGAQAPHPTHSR

SEQ ID NO: 80 - protein, 298 aa, Podospora anserina
STLESKLNPSYISGFVDGEGSFMLTIIKDNKYKLGWRVVCRFVISLHKKDLSLLNKIKEFFDVGNVFLMTKDSAQYRVESLKGLDLIINHFDKYPLITKKQADYKLFKMAHNLIKNKSHLTKEGLLELVAIKAVINNGLNNDLSIAFPGINTILRPDTSLPQILNPFWLSGFVDAEGCFSVVVFKSKTSKLGEAVKLSFILTQSNRDEYLIKSLIEYLGCGNTSLDPRGTIDFKVTNFSSIKDIIVPFFIKYPLKGNKNLDFTDFCEVVRLMENKSHLTKEGLDQIKKIRNRMNTNRK

SEQ ID NO: 81 - protein, 308 aa, Podospora anserina
RNYSHLIQKRVSLHPWFITGFTDGEGCFWIGVRRDPRNKLGWCVQAFFQIALHKKDEALLKAIQKYFGGIGAVKIRGDKCTFIVSSLSQITKVIIPHFDSYPLITNKLADYFLWKGIIEIMKAKKHLTLEGLNEIVRMKAQLNLGLSDELKTAFAETFSTSAIIKRILNKESIPHGMWMAGFTSGEGSFFVNIFKSSHHKVGYQIRLEFEIAQHIRDELLMLSFTEFFGCGIISRYSSDMVKFRCTKFSDISTIIVPFFKEYLPEGNKIKDFQDFCKVCELMENNAHLTEEGLNQIREIKARMNSFRK

SEQ ID NO: 82 - protein, 306 aa, Phaeosphaeria nodorum
KKFSTYRSTIVNPGVWSGLIDGEGSFSIIISKSKVRALGWRAELKFQLGLHTKDLNLLCLLQQHLGGIGSIHLAKNRDMVNYSIDSIKDLNNLILYLDKYPLLTQKAADFLLLKKAVELVNNKAHLTLEGLEKIVNIKASMNLGLSDMLISEFPGYVPVERPVINNDNVILNPYWISGFVSAEGNFDVRVPSTNSKLGYRVQLRFRISQHSRDLILMQKIVEYLGSGKIYKYAGKSSISLTIVDFKDITNILVPFFDENPIIGIKLHDYLDWCKIHSLMLNRSHLTVEGINSIRKIKSGMNTGRNF

SEQ ID NO: 83 - protein, 326 aa, Smittium culisetae
KEIQLFNLNPNWVTGFTQADGCFNITFTKKKPNQIRLRARFIISQHIKDELLILSIKNFFNCGIIIKNKKEIQYVVNSINDLINIIIPHFDKYPLKYGKYTSYLIFKNIIEKMKNKQHLTQKGLIDIINLTYIMNPLGKRKINKKELFEFLKIKDFSISDENNPYTDFSLSNKFLYQNEIDINFIGGLIQGDGCFNISFRKDLKIQAQLFIAQDMYSIELLSEIKKFFNCGNIIDKKVKMSIIEIKNINNLYNKILPLFNENLFFLDKLKQFIIFKQIVNLLYNKQHLTKDNKLKIVDLAYNMNNNGIKRKLTKEQFIQIIINKYK

SEQ ID NO: 84 - protein, 302 aa, Sordaria macrospora
SKGENSKLNPWAVVGFIDAEGSFMVRVRKNSKYKTGWLVVAIFSVTVDKKDLFLLESLKTFFGGLGSIKKSGNSTFSYRIESSEQLTKIILPFFDKYSLITEKLGDYLLFKKVLELMGTKEHLTQRGLEKIVSLKASINKGLSEELQAAFPQCVPTPRPEINNKLIPDPFWLAGFVSGDGSFKSILKKSESIKVGFQSILVFQITQHARDVKLMESLISYLGCGFIEKDSRGPWLYYTVTNFSDIQGKIIPFFHQYKIIGSKYGDYMDWCKIALIMQNKNHLTPEGLNEIRALKGGMNKGRL

SEQ ID NO: 85 - protein, 300 aa, Saccharomyces cerevisiae
FYSTSNIYVNKNINPWFLTGFIDGEGCFRISLTKVTRAIGWRVQLFFQINLHKKDIALLEDIRDYFGVGMIHKSGTNLVQYRIQTFDELSILINHLNDYPLVSQKKWDFELFKQAHELVKMNEHLNKEGILKIVSLKASLNLGLSEALKLAFPNVKNATKLTSSTVSIPDPHWFSGFTSAEGCFMVGIAKSKESTTGYQVYLSFIVTQHVRDELLLKCLIDYFSCGRLARKRDVYEYQVSKFSDVEKFIDFFDKYPILGEKSKDYLDFRTVSEIMRSKDHLTEVGVAKVRIIKQGMNRGR

SEQ ID NO: 86 - DNA, 22 bp, Artificial Sequence
  Leptographium Truncation
aatgctcctatacgacgtttag

SEQ ID NO: 87 - DNA, 22 bp, Artificial Sequence
  Leptographium Truncation
ctaaacgtcgtataggagcatt

SEQ ID NO: 88 - DNA, 22 bp, Artificial Sequence
  I-OnuI
taaaaggttgaataagtggaaa

SEQ ID NO: 89 - DNA, 22 bp, Artificial Sequence
  Complementary sequence
tttccacttattcaacctttta

SEQ ID NO: 90 - DNA, 22 bp, Artificial Sequence
  I-LtrI
ctaaacgtcgtataggagcatt

SEQ ID NO: 91 - DNA, 22 bp, Artificial Sequence
  Complementary sequence
aatgctcctatacgacgtttag

SEQ ID NO: 92 - DNA, 28 bp, Artificial Sequence
  I-GpiI
taaaaattttcctgtatatgacttaaat

SEQ ID NO: 93 - DNA, 28 bp, Artificial Sequence
  Complementary sequence
atttaagtcatatacaggaaaattttta

SEQ ID NO: 94 - DNA, 22 bp, Artificial Sequence
  GpeI
tttccgcttattcaacccttta

SEQ ID NO: 95 - DNA, 22 bp, Artificial Sequence
  GpiI
ttttcctgtatatgacttaaat

SEQ ID NO: 96 - DNA, 22 bp, Artificial Sequence
  MpeI
tagataaccataagtggctaat

SEQ ID NO: 97 - DNA, 22 bp, Artificial Sequence
  PanII
gctcctcataatccttatcaag

SEQ ID NO: 98 - DNA, 22 bp, Artificial Sequence
  GzeI
gcccctcataacccgtatcaag

SEQ ID NO: 99 - DNA, 22 bp, Artificial Sequence
  SscI
aggtaccctttaaacctattaa

SEQ ID NO: 100 - DNA, 22 bp, Artificial Sequence
  AabI
aggtaccctttaaacctactaa

SEQ ID NO: 101 - DNA, 22 bp, Artificial Sequence
  PnoII
cctttggttatgaggatcttca

SEQ ID NO: 102 - DNA, 22 bp, Artificial Sequence
  GzeII
tttgtaccaatatggtacccat

SEQ ID NO: 103 - DNA, 22 bp, Artificial Sequence
  CapIII
atggccttaatattgtgggcta

SEQ ID NO: 104 - DNA, 22 bp, Artificial Sequence
  LtrII
ttaaataacatacttcactact

SEQ ID NO: 105 - DNA, 22 bp, Artificial Sequence
  SmaI
taatggaggataaattgttcat

SEQ ID NO: 106 - DNA, 50 bp, Artificial Sequence
  GpiI Target plasmid cleavage
tagccatatgttaaaaattttcctgtatatgacttaaattctcgagtcta

SEQ ID NO: 107 - DNA, 50 bp, Artificial Sequence
  GpiI Target plasmid cleavage
tagactcgagaatttaagtcatatacaggaaaatttttaacatatggcta

SEQ ID NO: 108 - protein, 300 aa, Artificial Sequence
  xOnuRound2
DLSTSINPWILTGFADAEGSFLLRIRKNNKSNVGWSTELGFQITLHNKDKSILENIQNTWKVGVIANSGDNAVSLKVTSFKDLEVIIDHFDKYPLITQKQADYKLFKKAFEVMKNKNHLTEEGINQLVTIKASLNWGLSNSLKKAFPNNIESASKNNSSVNGNIPNFNWLAGFTSGEGCFFVNLIKSKSKLGVQVQLVFSITQHTRDQELMKSLISYLNCGYIKEKNKSEFSWLDFVVTKFSDINEKIIPVFQENKLIGVKLQDFQDWCKVAKLIDEKKHLTSEGLEKIRKIKNNMNRGR

SEQ ID NO: 109 - protein, 304 aa, Artificial Sequence
  xLtrRound2
DLSTSINPWTITGFADAEGSFMLTVSKNNKRNTGWSVRPRFRIGLHNKDKSILENIQNYLKAGIITSDKDARIRFESLKDLEVVIDHFDKYPLITQKQADYKLFKKAFELIKNKNHLTEEGLNQILTLKASLNLGLSNSLKKAFPNNIESASKNNSSVNGNIPNSNWVAGFTAGEGSFYIRIAKNSTLKTGYQVQLVFQITQDTRDQELMKSLISYLNCGNIRIRKYKGSEGIHDTCVDLVVTKLSDINEKIIPFFQENKIIGVKLQDYQDWCKVAKLIDEKKHLTSEGLEKIRKIKNNMNRGR

SEQ ID NO: 110 - DNA, 80 bp, Artificial Sequence
  MAO-B gene deletion sequence
ctgggttggtccaacataggatcctccaaggtccacatatttaaccttttggttctgttttcccataggaaaaaattaaa

SEQ ID NO: 111 - DNA, 79 bp, Artificial Sequence
  MAO-B gene deletion sequence
ctgggttggtccaacataggatcctccaaggtccacattttaaccttttggttctgttttcccataggaaaaaattaaa

SEQ ID NO: 112 - DNA, 78 bp, Artificial Sequence
  MAO-B gene deletion sequence
ctgggttggtccaacataggatcctccaaggtccacatatttccttttggttctgttttcccataggaaaaaattaaa

SEQ ID NO: 113 - DNA, 75 bp, Artificial Sequence
  MAO-B gene deletion sequence
ctgggttggtccaacataggatcctccaaggtccacaaaccttttggttctgttttcccataggaaaaaattaaa

SEQ ID NO: 114 - DNA, 72 bp, Artificial Sequence
  MAO-B gene deletion sequence
ctgggttggtccaacataggatcctccaaggtccacatatttggttctgttttcccataggaaaaaattaaa

SEQ ID NO: 115 - DNA, 71 bp, Artificial Sequence
  MAO-B gene deletion sequence
ctgggttggtccaacataggatcctccaaatttaaccttttggttctgttttcccataggaaaaaattaaa

SEQ ID NO: 116 - DNA, 71 bp, Artificial Sequence
  MAO-B gene deletion sequence
ctgggttggtccaacataggatcctccaaggtccaccttttggttctgttttcccataggaaaaaattaaa

SEQ ID NO: 117 - DNA, 66 bp, Artificial Sequence
  MAO-B gene deletion sequence
ctgggttggtccaacataggatcctccaaggtccacatatttattttcccataggaaaaaattaaa

SEQ ID NO: 118 - DNA, 65 bp, Artificial Sequence
  MAO-B gene deletion sequence
ctgggttggtccaacataggatcctccaaccttttggttctgttttcccataggaaaaaattaaa

SEQ ID NO: 119 - DNA, 28 bp, Artificial Sequence
  PCR Primer
ggtctaaacgtcgtataggagcatttgg

SEQ ID NO: 120 - DNA, 28 bp, Artificial Sequence
  PCR Primer
caaatgctcctatacgacgtttagaccc

SEQ ID NO: 121 - DNA, 22 bp, Artificial Sequence
  PCR Primer
gttccagactacgctctgcagg

SEQ ID NO: 122 - DNA, 20 bp, Artificial Sequence
  PCR Primer
gtgctgcaaggcgattaagt